SEQUENCE LISTING (7 sequences)

SEQ ID NO: 1 - DNA, 1200 bp, Homo sapiens
atgaggcccgagcgtccccggccgcgcggcagcgcccccggcccgatggagaccccgccgtgggacccagcccgcaacgactcgctgccgcccacgctgaccccggccgtgcccccctacgtgaagcttggcctcaccgtcgtctacaccgtgttctacgcgctgctcttcgtgttcatctacgtgcagctctggctggtgctgcgttaccgccacaagcggctcagctaccagagcgtcttcctctttctctgcctcttctgggcctccctgcggaccgtcctcttctccttctacttcaaagacttcgtggcggccaattcgctcagccccttcgtcttctggctgctctactgcttccctgtgtgcctgcagtttttcaccctcacgctgatgaacttgtacttcacgcaggtgattttcaaagccaagtcaaaatattctccagaattactcaaataccggttgcccctctacctggcctccctcttcatcagccttgttttcctgttggtgaatttaacctgtgctgtgctggtaaagacgggaaattgggagaggaaggttatcgtctctgtgcgagtggccattaatgacacgctcttcgtgctgtgtgccgtctctctctccatctgtctctacaaaatctctaagatgtccttagccaacatttacttggagtccaagggctcctccgtgtgtcaagtgactgccatcggtgtcaccgtgatactgctttacacctctcgggcctgctacaacctgttcatcctgtcattttctcagaacaagagcgtccattcctttgattatgactggtacaatgtatcagaccaggcagatttgaagaatcagctgggagatgctggatacgtattatttggagtggtgttatttgtttgggaactcttacctaccaccttagtcgtttatttcttccgagttagaaatcctacaaaggaccttaccaaccctggaatggtccccagccatggattcagtcccagatcttatttctttgacaaccctcgaagatatgacagtgatgatgaccttgcctggaacattgcccctcagggacttcagggaggttttgctccagattactatgattggggacaacaaactaacagcttcctggcacaagcaggaactttgcaagactcaactttggatcctgacaaaccaagccttgggtag

SEQ ID NO: 2 - DNA, 755 bp, Homo sapiens
catcagttaacagttttatggacgattcctcagatgaaaagcttcagaaaagcatagtgacagctgaatttttagggcacttttccttaagaaatagaacttgatttttatttgttacaggtttccaatggccccataggaataagcaataatgtagactgataaacccttattttagtactaaagagggagccttgctatttcagtgggtataatttaaactttttaaagaaaatctgtacttttataaagatgtattttgtataacttaaataataatgctaaagtatactagggtttttttttcttgagaatgttactgcaatcatgttgtagtttgcacagacttttatgcataattcactttaaaaatatagaatatatggtctaatagttttttaaagcttttggactaaagtattccacaaatcttacctctttaggtcactgatggtcactccgattctgagtgccacattggtagactcctaaaatacagttgacaacttagccaattgcaactccagtgttgataattaaaatgaaatggtaaagcagcagactgtaaggtctttagagattttttttttaaggttcaggccgtaggttcctcaaggaatctcttaagttttgcccaaagactggtacttcctttcagtagggcgctaatgtatacacattaatgataagttgataacattaaaaatgtagctgacttatcctattaaacctcctctgctatgttcacagaaaaa

SEQ ID NO: 3 - RNA, 19 nt, Artificial Sequence
  GPR137B siRNA #1
ggacuaaaguauuccacaa

SEQ ID NO: 4 - RNA, 19 nt, Artificial Sequence
  GPR137B siRNA #2
gcaauaauguagacugaua

SEQ ID NO: 5 - protein, 399 aa, Homo sapiens
MRPERPRPRGSAPGPMETPPWDPARNDSLPPTLTPAVPPYVKLGLTVVYTVFYALLFVFIYVQLWLVLRYRHKRLSYQSVFLFLCLFWASLRTVLFSFYFKDFVAANSLSPFVFWLLYCFPVCLQFFTLTLMNLYFTQVIFKAKSKYSPELLKYRLPLYLASLFISLVFLLVNLTCAVLVKTGNWERKVIVSVRVAINDTLFVLCAVSLSICLYKISKMSLANIYLESKGSSVCQVTAIGVTVILLYTSRACYNLFILSFSQNKSVHSFDYDWYNVSDQADLKNQLGDAGYVLFGVVLFVWELLPTTLVVYFFRVRNPTKDLTNPGMVPSHGFSPRSYFFDNPRRYDSDDDLAWNIAPQGLQGGFAPDYYDWGQQTNSFLAQAGTLQDSTLDPDKPSLG

SEQ ID NO: 6 - protein, 475 aa, Homo sapiens
MLWAVRTRCYVVKAQLGPTVALEGRAPRAPGPSCLGNGNCQRPGPITSRNVTRASLPDMESNLSGLVPAAGLVPALPPAVTLGLTAAYTTLYALLFFSVYAQLWLVLLYGHKRLSYQTVFLALCLLWAALRTTLFSFYFRDTPRANRLGPLPFWLLYCCPVCLQFFTLTLMNLYFAQVVFKAKVKRRPEMSRGLLAVRGAFVGASLLFLLVNVLCAVLSHRRRAQPWALLLVRVLVSDSLFVICALSLAACLCLVARRAPSTSIYLEAKGTSVCQAAAMGGAMVLLYASRACYNLTALALAPQSRLDTFDYDWYNVSDQADLVNDLGNKGYLVFGLILFVWELLPTTLLVGFFRVHRPPQDLSTSHILNGQVFASRSYFFDRAGHCEDEGCSWEHSRGESTRCQDQAATTTVSTPPHRRDPPPSPTEYPGPSPPHPRPLCQVCLPLLAQDPGGRGYPLLWPAPCCSCHSELVPSP

SEQ ID NO: 7 - protein, 429 aa, Homo sapiens
MRVSVPGPAAAAAPAAGREPSTPGGGSGGGGAVAAASGAAVPGSVQLALSVLHALLYAALFAFAYLQLWRLLLYRERRLSYQSLCLFLCLLWAALRTTLFSAAFSLSGSLPLLRPPAHLHFFPHWLLYCFPSCLQFSTLCLLNLYLAEVICKVRCATELDRHKILLHLGFIMASLLFLVVNLTCAMLVHGDVPENQLKWTVFVRALINDSLFILCAISLVCYICKITKMSSANVYLESKGMSLCQTVVVGSVVILLYSSRACYNLVVVTISQDTLESPFNYGWDNLSDKAHVEDISGEEYIVFGMVLFLWEHVPAWSVVLFFRAQRLNQNLAPAGMINSHSYSSRAYFFDNPRRYDSDDDLPRLGSSREGSLPNSQSLGWYGTMTGCGSSSYTVTPHLNGPMTDTAPLLFTCSNLDLNNHHSLYVTPQN

(19) United States
(12) Patent Application Publication
(10) Pub. No.: US 2016/0138027 A1
(43) Pub. Date: May 19, 2016

(54) TREATMENT OF DISEASES AND CONDITIONS ASSOCIATED WITH DYSREGULATION OF MAMMALIAN TARGET OF RAPAMYCIN COMPLEX 1 (MTORC1)

(71) Applicant: THE BOARD OF TRUSTEES OF THE LELAND STANFORD JUNIOR UNIVERSITY, Palo Alto, CA (US)

(72) Inventors: Lin Gan, Palo Alto, CA (US); Tobias Meyer, Menlo Park, CA (US)

(21) Appl. No.: 14/775,686

(22) PCT Filed: Mar. 14, 2014
(86) PCT No.: PCT/US14/29164

§ 371 Date: Sep. 12, 2015

Related U.S. Application Data
(60) Provisional application No. 61/785,747, filed on Mar. 14, 2013.

(51) Int. Cl. C12N 15/113; C07K 16/28

(52) U.S. Cl. CPC C12N 15/1138; C07K 16/28; C07K 2317/76; C12N 2310/111; C12N 2320/30; C12N 2310/14

(57) ABSTRACT

Compositions and methods for treating diseases and conditions associated with dysregulation of mammalian target of rapamycin complex 1 (mTORC1) are disclosed. The invention is based in part on the discovery that protein mediator of amino acid signaling to mTOR (MORTOR) is involved in amino acid-induced translocation of mTORC1 to lysosomes where MORTOR forms a signaling complex with mTORC1, Ragulator, and Rag GTPases, which controls protein synthesis. In particular, the invention relates to the use of antagonists of MORTOR for treating diseases and conditions associated with dysregulation of mTORC1. Downregulation of expression of MORTOR by RNA interference has been shown to reduce cell proliferation of cancerous cells and may be useful for treating cancer.

### STATEMENT REGARDING FEDERALLY SPONSORED RESEARCH OR DEVELOPMENT

This invention was made with Government support under contract CA120732 awarded by the National Institutes of Health. The Government has certain rights in this invention.

### TECHNICAL FIELD

The present invention pertains generally to compositions and methods for treating diseases and conditions associated with dysregulation of mammalian target of rapamycin complex 1 (mTORC1). In particular, the invention relates to the use of antagonists of the protein mediator of amino acid signaling to mTOR (MORTOR) for treating diseases and conditions associated with dysregulation of mTORC1.

### BACKGROUND

Mammalian cells control their size, proliferation activity, and autophagy rates by monitoring the concentration of amino acids. Amino acids can be sensed in lysosomes by a poorly understood signaling pathway involving mTORC1, an evolutionarily conserved signaling complex that includes the protein kinase mTOR and an adaptor protein raptor. Mammalian TORC1 controls cell growth by integrating multiple upstream signals including nutrients, growth factors, oxygen, and stress (Inoki et al. (2002) Nat. Cell Biol. 4:648-657; Ma et al. (2005) Cell 121:179-193; Roux et al. (2004) Proc. Natl. Acad. Sci. USA 101:13489-13494; Inoki et al. (2003) Cell 115:577-590; Gwinn et al. (2008) Mol. Cell 30:214-226; Liu et al. (2006) Mol. Cell 21:521-531; Brugarolas et al. (2004) Genes Dev. 18:2893-2904; Feng et al. (2005) Proc. Natl. Acad. Sci. USA 102:8204-8209; and Lee et al. (2007) Cell 130:440-455). The complex is often described as a master regulator of cell growth because it induces important anabolic processes such as ribosome biogenesis, translation, lipid biosynthesis and transcription (Ma et al. (2009) Nat. Rev. Mol. Cell Biol. 10:307-318; Porstmann et al. (2008) Cell Metab. 8:224-236; Huffman et al. (2002) Proc. Natl. Acad. Sci. USA 99:1047-1052; Chen et al. (2008) J. Exp. Med. 205:2397-2408; and Cunningham et al. (2007) Nature 450:736-740). Mammalian TORC1 also suppresses autophagy, a process that breaks down and recycles cell mass when nutrients become limited (Chan (2009) Science Signaling, 2(84):pe51). A diverse number of proteins that control mTORC1 are potential drug targets due to the central role mTOR plays not only in metabolic dysfunction but also in cancer, diabetes, aging, and neurodegenerative diseases (Zoncu et al. (2011) Nat. Rev. Mol. Cell Biol. 12:21-35).

### SUMMARY

The present invention is based, in part, on the discovery that MORTOR is involved in amino acid-induced translocation of mTORC1 to lysosomes where MORTOR forms a signaling complex with mTORC1, Ragulator, and Rag GTPases, which controls protein synthesis. Antagonists of MORTOR can be used for treatment of diseases and conditions associated with dysregulation of mTORC1.

In one aspect, the invention includes a method for treating a subject having a disease or condition associated with dysregulation of mammalian target of mTORC1, the method comprising administering a therapeutically effective amount of an antagonist of MORTOR to the subject. Diseases and conditions associated with dysregulation of mTORC1 that can be treated by methods of the invention include, but are not limited to cancer, obesity, a metabolic disease or condition, a neurodegenerative disease or condition, or aging.

Exemplary antagonists of MORTOR include antisense oligonucleotides or inhibitory RNA molecules, such as small interfering RNAs (siRNAs), short hairpin RNAs (shRNAs), microRNAs (miRNAs), Piwi-interacting RNAs (piRNAs), and small nuclear RNAs (snRNAs), and Clustered Regularly Interspaced Short Palindromic Repeats (CRISPR) interference (CRISPRi) systems comprising guide crRNAs and nuclease-deficient Cas (e.g., dCas9) protein that downregulate expression of MORTOR, antibodies that specifically bind to MORTOR that interfere with its interactions with other proteins (e.g., Raptor, mTOR, or Rag GTPases), and amino acid synthesis inhibitors. In one embodiment, the antagonist is an antibody that specifically binds to a MORTOR protein comprising a sequence selected from the group consisting of SEQ ID NOS:5-7 or a variant thereof displaying at least about 50-99% sequence identity thereto, including any percent identity within this range, such as 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto. In certain embodiments, the antagonist is an antagonist of GPR137A, GPR137B, or GPR137C, or any combination thereof.

By “therapeutically effective dose or amount” of an antagonist of MORTOR is intended an amount that, when administered, as described herein, brings about a positive therapeutic response, such as improved recovery from a disease or condition associated with dysregulation of mTORC1. In one embodiment, the disease or condition associated with dysregulation of mTORC1 is cancer and a “therapeutically effective dose or amount” of an antagonist of MORTOR is an amount that has anti-tumor activity.

In another aspect, the invention includes a method of inhibiting MORTOR in a subject, the method comprising administering an effective amount of an antagonist of MORTOR to the subject.

In another aspect, the invention includes a method for inhibiting MORTOR in a cell by introducing an effective amount of an antagonist of MORTOR into the cell. In one embodiment, the cell is a cancerous cell, wherein inhibiting MORTOR decreases cell proliferation.

In another aspect, the invention includes a method of decreasing translocation of mTORC1 to lysosomes in a cell, the method comprising introducing an effective amount of an antagonist of MORTOR into the cell.

In another aspect, the invention includes a method for treating cancer comprising administering to a subject in need thereof a therapeutically effective amount of a composition comprising at least one antagonist of MORTOR. In certain embodiments, the antagonist downregulates expression of MORTOR through RNA interference (RNAi). In one embodiment, the antagonist is an antisense oligonucleotide or an inhibitory RNA molecule, such as a miRNA, siRNA, shRNA, piRNA, or snRNA comprising a nucleotide sequence sufficiently complementary to a MORTOR target mRNA sequence to bind to and downregulate expression of the MORTOR mRNA. In one embodiment, the antagonist is an antisense oligonucleotide or an inhibitory RNA molecule that binds to a target MORTOR mRNA comprising a sequence selected from the group consisting of SEQ ID NO:1 and SEQ ID NO:2. In one embodiment, the antagonist is an siRNA comprising a sequence selected from the group consisting of SEQ ID NO:3 and SEQ ID NO:4. In other embodiments, the antagonist downregulates expression of MORTOR through CRISPR interference (CRISPRi). In one embodiment, the CRISPRi system comprises a crRNA that binds to a target MORTOR mRNA sequence. In one embodiment, the target MORTOR mRNA comprises a sequence selected from the group consisting of SEQ ID NO:1 and SEQ ID NO:2.

In another aspect, the invention includes compositions for treatment of a disease or condition associated with dysregulation of mTORC1. The compositions may comprise one or more antagonists of MORTOR. In certain embodiments, compositions may further comprise a pharmaceutically acceptable carrier. Compositions may be administered by any suitable method, including but not limited to subcutaneously, intraperitoneally, intramuscularly, intravenously, orally, intralesionally, or intra-arterially. In one embodiment, compositions are administered locally into a tumor or cancerous cells.

In another aspect, the invention includes a kit comprising one or more antagonists of MORTOR and instructions for treating diseases and conditions associated with dysregulation of mTORC1. In certain embodiments, the kit comprises one or more amino acid synthesis inhibitors, MORTOR specific antibodies, or antisense oligonucleotides, inhibitory RNAs, CRISPRi Cas/crRNAs, or recombinant polynucleotides or vectors encoding them that downregulate expression or decrease activity of MORTOR. One or more antagonists of MORTOR may be combined in a pharmaceutical composition. The kit may further comprise means for delivering the composition to a subject.

In another aspect, the invention includes use of an antagonist of MORTOR in the manufacture of a medicament for treating a disease or condition associated with dysregulation of mTORC1. In one embodiment, the invention includes use of an siRNA comprising a sequence selected from the group consisting of SEQ ID NO:3 and SEQ ID NO:4 in the manufacture of a medicament for treating cancer.

In another aspect, the invention includes use of an antagonist of MORTOR in a method for treating a disease or condition associated with dysregulation of mTORC1.

In another aspect, the invention includes a method of screening an agent for the ability to treat an individual having a disease or condition associated with dysregulation of mTORC1, the method comprising: a) treating a cell expressing a MORTOR protein and mTOR with the agent; and b) measuring MORTOR protein activity in the cell; wherein a decrease in MORTOR protein activity in the cell treated with the agent compared to a cell untreated with the agent indicates that the agent will treat an individual having a disease or condition associated with dysregulation of mTORC1. The agent may be a small molecule, a nucleic acid, an antibody, or a polypeptide. The agent may affect the activity of one or more isoforms of MORTOR, including GPR137A, GPR137B, or GPR137C, or any combination thereof.

In one embodiment, the activity of MORTOR is measured by determining the amount of mTORC1 localization to lysosomes in the cell, wherein a decrease in mTORC1 localization to lysosomes in the cell treated with the agent compared to a cell untreated with the agent indicates that the agent will treat an individual having a disease or condition associated with dysregulation of mTORC1.

In another embodiment, the activity of MORTOR is measured by determining the amount of phosphorylation of ribosomal S6 in the cell, wherein a decrease in the amount of phosphorylation of ribosomal S6 in the cell treated with the agent compared to a cell untreated with the agent indicates that the agent will treat an individual having a disease or condition associated with dysregulation of mTORC1.

In another embodiment, the activity of MORTOR is measured by determining the amount of inhibition of authophagy in the cell, wherein an increase in the amount of autophagy in the cell treated with the agent compared to a cell untreated with the agent indicates that the agent will treat an individual having a disease or condition associated with dysregulation of mTORC1.

In another embodiment, the agent is screened for the ability to treat an individual having cancer, wherein the cell is a cancer cell, and the MORTOR protein activity is measured by assessing cell growth and proliferation.

In another embodiment, the agent is screened for the ability to treat an individual having a neurodegenerative disease, wherein the cell is a neuron, and the MORTOR protein activity is measured by assessing cell death.

These and other embodiments of the subject invention will readily occur to those of skill in the art in view of the disclosure herein.

### BRIEF DESCRIPTION OF THE FIGURES

FIGS. 1A-1G show that a genome-wide human siRNA screen identified candidate regulators of amino acid stimulated rpS6 phosphorylation. FIG. 1A shows the mTORC1 signaling pathway. Phosphorylation of rpS6 was used as the amino acid sensitive readout for screening. FIG. 1B shows (Left top) the protocol for a microscopy-based assay for amino acid sensing and (Right) rpS6 phosphorylation immunofluorescence images of fibroblast Hs68 cells reverse-transfected with siRNAs in a 384 well format. The effects of siRNAs targeting the mTOR pathway components Rheb and TSC2 are shown. Quantification (Left bottom) of siRNA-mediated changes in integrated single cell rpS6 immunofluorescence intensities are also shown (n>300 cells per well, error bars are ±1 S.D). FIG. 1C shows the sequential selection strategy for candidate regulatory genes. FIG. 1D shows that a genome-wide screen identified siRNAs that increased or decreased rpS6 phosphorylation. The inset top shows replicate experiments showing that results from the primary screen are repeatable. The inset right shows a scatter plot of the Z′ factor of siRNA effects for the 230 384-well plates used for the screen. Extreme values were removed for calculation of the Z′ factor. FIG. 1E shows additional validation experiments comparing effects of Giardia-diced siRNA pools to synthetic siRNA results from a primary screen. Data points labeled in light gray are known mTOR regulators; labeled in black are ribosomal subunits that increased rpS6 when targeted by siRNA. FIG. 1F shows that pathway analysis (Ingenuity) shows statistical enrichment of hits from the screen in a subset of pathways. We used only siRNAs that were also present in the top 881 group of the initial genome-wide siRNA screen to obtain an unbiased group for the analysis (307 instead of the 422 validated genes). FIG. 1G shows that the primary screen identified previously characterized amino acid/TOR signaling pathway regulatory components (light gray and dark gray for negative and positive regulators, respectively).

FIGS. 2A-2E depict the identification of a subset of candidate regulators of amino acid triggered mTORC1 translocation to lysosomes. FIG. 2A shows an automated microscopy-based mTORC1 translocation assay. At Left is a diagram showing mTORC1 translocation in response to increased amino acids. At Right are confocal images of primary Hs68 fibroblasts co-stained for Lamp2 and mTOR. Hs68 cells were transfected with siRNAs, starved of amino acids and serum for 4 hours and re-stimulated with 20 μg/ml cycloheximide for 5 minutes. Cycloheximide induces a transient rise in intracellular amino acid concentrations by inhibiting translation. Cells were co-stained with antibodies against endogenous mTOR and Lamp2. FIG. 2B shows an image-based correlation analysis demonstrating amino acid stimulated and RagC-dependent co-localization of mTOR and Lamp2 (n=6 wells from 4 independent experiments). All error bars are ±1 S.D. of the population average. FIG. 2C shows a mTORC1 translocation assay, which identified several candidate siRNAs that decreased mTOR Lamp2 co-localization as well as rpS6 phosphorylation. Duplicate assays were performed in 96 well plates using the strongest single siRNA from FIG. 1. Scores were normalized per plate. Hits below the dashed line were selected for further validation with a 2^{nd} and 3^{rd} siRNA (as well as a few interesting candidates with lower scores confirmed by Giardia-dicer siRNA). As expected, Rheb, RPS6KB2, TSC2 siRNAs changed rpS6 phosphorylation without altering mTORC1 translocation (brown solid circles). Hits selected for further validation are marked with a closed circle if they were confirmed by a 2^{nd} independent siRNAs (open circle if not confirmed). FIG. 2D shows a ranked list of 25 candidate regulators that reduced cycloheximide-induced mTOR translocation confirmed by at least 2 independent siRNAs. ACADL* up-regulates rpS6 phosphorylation when knocked down. FIG. 2E shows co-evolution of mTORC1, Ragulator, RagGTPases and MORTOR.

FIGS. 3A-3F show that MORTOR regulates amino acid-induced mTORC1 translocation and activation. FIG. 3A shows that siRNA knockdown of MORTOR reduces amino acid-induced mTORC1 translocation and S6 phosphorylation. At Left, confocal images are shown of Hs68 cells co-immunostained with the lysosomal marker Lamp2 and mTOR antibodies. Hs68 cells transfected with different single MORTOR siRNAs were stimulated for 5 minutes with cycloheximide after 4 hours of amino acid and serum starvation. At Right is shown the quantification of changes in rpS6 phosphorylation and mTOR/Lamp2 co-localization. Effects of Rheb and RRAGC siRNAs are shown for comparison as they are known to interfere with mTORC1 activation and translocation, respectively. Values were normalized to Scramble siRNAs in each plate. FIG. 3B shows that MORTOR-Venus over-expression increases amino acid-induced mTOR translocation. Cells were fixed and stained with antibodies against Lamp2 and mTOR. At Right is shown the quantification of mTOR puncta (>1000 cells). Error bars±1 S.E.M of the average of the population and P values are calculated in comparison to untransfected wells of the same well. n>5,000. FIG. 3C shows at Top, the rescue of mTORC1 translocation by MORTOR-Venus expression in HeLa cells treated with a 3′UTR siRNA against MORTOR. At Bottom is shown the quantification of mTOR puncta formation comparing normalized MORTOR-Venus and Lamp1-GFP overexpression. N=6 wells from 3 independent experiments. Error bars±1 SD of the average of the population. FIG. 3D shows that MORTOR knockdown enhanced autophagy. Analysis of LC3-GFP puncta in HeLa cells co-transfected with the autophagy marker LC3-GFP together with either Scramble or MORTOR siRNA (n>500 cells per). FIG. 3E shows that MORTOR-Venus expression induced phosphorylation of 4EBP1 at sites 37/46 even during amino acid starvation (N=2). FIG. 3F shows a sensitivity comparison. Amino acid addition enhances a pre-existing basal mTOR-mediated phosphorylation of 4EBP1 at 37/46. The basal phosphorylation can be blocked by the mTOR inhibitor TORIN. Expression of MORTOR-Venus increased basal 4EBP1 close to the maximal stimulated level. Error bars±1 S.E.M of the average of the population and P values are calculated in comparison to untransfected wells of the same well (n>5,000). FIG. 3G shows that GPR137 and GPR137C isoforms localized at lysosomes. FIG. 3H shows that GPR137 overexpression enhanced mTOR translocation to lysosomes. FIG. 3I shows that GPR137c overexpression enhanced mTOR translocation to lysosomes.

FIGS. 4A-4F show that MORTOR is localized in lysosomal membranes. FIG. 4A shows that expressed MORTOR-Venus co-localized with endogenous Lamp2 and mTOR with triple color staining HeLa cells were transfected with MORTOR-Venus and fixed and stained with Lamp2 and mTOR antibodies. FIG. 4B shows that MORTOR-Venus was exclusively found in membrane fractions of HEK293T cells and co-localized with the membrane of enlarged lysosomes in NPC-1 deficient CHO cells. Integral membrane and cytosolic fractions of HEK293T cells were compared. FIG. 4C shows that endogenous MORTOR co-localized with lysosomes in mouse kidney tissue sections where MORTOR was significantly enriched. Tissue sections were stained with antibody staining endogenous MORTOR and Lamp1. FIG. 4D shows CHO cells stably expressing a NPC-1 mutant, transfected with MORTOR-Venus and luminal staining with LysoTracker Red.

FIGS. 5A-5D show that MORTOR acts upstream of Rag GTPases in regulation of mTORC1 translocation. FIG. 5A shows experiments to determine whether MORTOR acts upstream of RagA/C complexes. FIG. 5B shows that expression of constitutively active RagA/C heterodimer (RagA66L/T75N) rescued MTORC1 translocation in cells treated with siRNAs targeting MORTOR. HeLa cells were treated with control Scramble siRNA or MORTOR siRNA. At Left is shown HeLa cells transfected with MORTOR siRNA and stained for mTOR. Cells co-expressing RagA66L/T75N are marked. At Right is shown the analysis of the change in MTORC1 translation by CA RagA/C with and without MORTOR knockdown (N=4 wells from 2 independent experiments. Error bars±1 SD of the average of the population (FIG. 5B)). FIG. 5C shows that RRAGC is required for MORTOR-mediated mTOR puncta formation. At Left, it is shown that HeLa cells transfected with RRAGC siRNA do not show endogenous MTORC1 translocation when MORTOR-Venus is over-expressed. At Right is shown a bar graph analysis showing that MORTOR-Venus or HA conjugated MORTOR both lose their effect on mTORC1 translocation when RagC is knocked down (N=4 wells from 2 independent experiments. Error bars±1 SD of the average of the population). FIG. 5D shows that dominant negative RagA/C complex (using RagA21L/C) reduces mTOR recruitment in cells co-expressing MORTOR-Venus (Error bars±1 S.E.M of the average of the population and P values are calculated in comparison to untransfected wells of the same well. n>500). FIG. 5E shows that neither MORTOR knockdown or MORTOR-Venus expression significantly alters endogenous RRAGC localization or expression in either starvation or amino acid conditions. At Left is shown that HeLa cells treated with control Scramble or MORTOR siRNA with and without amino acids showed similar lysosomal localization of endogenous RRAGC. At Bottom is shown that RagC localization was also not changed by MORTOR in the presence of serum and amino acid (N=4 wells from 2 independent experiments. Error bars±1 SD of the average of the population).

FIGS. 6A and 6B show that MORTOR interacts with RagA, Raptor and mTOR in response to amino-acid stimulation. FIG. 6A shows plausible mechanisms by which MORTOR may control amino acid induced mTORC1 translocation and activation. FIG. 6B shows that FLAG conjugated MORTOR interacts with endogenous mTOR, Raptor and RagA following amino acid stimulation. HEK293T cells were transfected with either MORTOR-3×FLAG or FLAG alone for 22 or 42 hours later. Cells were then starved of amino acids and serum for 4 hours and re-stimulated with amino acids for 5 minutes or left untreated. Cells were cross-linked and harvested and FLAG immunoprecipitation was used to test for interactions with endogenous proteins. FIG. 6C shows that Rictor, Lamp2, p62, ATP6V1A, Rheb, p14 and p18 did not co-immunoprecipitate with MORTOR-3×FLAG. HEK293T cells were transfected with either MORTOR-3×FLAG, MORTOR-1×FLAG (CMV), or FLAG only and treated as in FIG. 6A.

FIGS. 7A-7F show evidence that MORTOR functions as an amino acid regulated scaffold that retains the Rag/mTORC1 complex at the lysosomal membrane. FIG. 7A shows at Left and middle, high but not low levels of MORTOR expression render cells insensitive to amino acids starvation. HeLa cells transfected with MORTOR-Venus were starved of amino acids and serum for 4 hours, then fixed and stained for mTOR. Cells were binned using the natural log of the Venus fluorescence intensity. At Right, it is shown that as MORTOR-FLAG expression increases from 22 to 42 hours after transfection, the amino acid stimulation requirement for the interaction between MORTOR and TOR and RagA is mostly lost. FIG. 7B shows mistargeting of Ragulator to mitochondria. Targeting of a core Ragulator component p18 to mitochondria instead of lysosomes suppresses endogenous MTORC1 translocation (p18 anchors RagA/C). Overexpression of either MORTOR-Venus and/or a constitutively active RagA/C complex synergistically restore mTOR puncta formation in these cells. FIG. 7C shows interpretation of mitochondrial mistargeting experiments. FIG. 7D shows the interaction between MORTOR-3×FLAG and mTOR, but not between MORTOR and Raptor or RagA, can be observed in the absence of cross-linking FIG. 7E shows that MORTOR-3×FLAG interacts with mTOR through both, the mTOR N-terminal and C-terminal regions. FIG. 7F shows a model of lysosomal amino acid sensing, which is mediated by formation of a MORTOR-Rag-mTORC1 signaling complex that stabilizes the RagA-GTP/raptor interaction.

FIGS. 8A-8D show that a primary screen is specifically enriching for mTOR pathway related genes. FIG. 8A shows that rpS6 phosphorylation is a robust and sensitive readout of the mTORC1 activation by amino acids and various inhibitors. FIG. 8B shows a Chi-square test of the primary screen hits compared to diced hits and to literature hits shows non-random enrichment of low pS6 hits and functionally important hits. FIG. 8C shows at Left, the strongest single in each pool dominates the pool behavior in the primary screen. The deconvolution value of the strongest single is plotted on the X-axis against the primary screen value on the Y-axis. At Right, it is shown that the average of the second and third strongest singles is an independent measurement from the primary screen value. The deconvolution value of the average of the second and third strongest singles is plotted on the X-axis against the primary screen value on the Y-axis. FIG. 8D shows an Ingenuity network analysis of 307 deconvolution hits showing enrichment of glycine cleavage complex.

FIG. 9 shows single cell measurements of mTOR translocation and rpS6 phosphorylation. Cells, treated with either siControl (left) or siRheb (right) in the presence of amino acids, were stained and fixed with an endogenous mTOR antibody and an endogenous phospho-rpS6 antibody conjugated to Alexa 488. The mTOR puncta intensity ratio and integrated intensity of phospho-rpS6 were quantified for individual cells, and plotted on the Y- and X-axis, respectively.

FIG. 10 shows that two independent synthetic siRNAs against MORTOR caused significant knockdown of expression as measured by qPCR. Transcript levels of MORTOR remaining 68 to 72 hours after transfection of Hs68 cells with the siRNAs (20 nm) that caused the strongest and the second strongest mTOR phenotype are shown (n=6 from 3 independent experiments. Error bars represent the ±1 S.D of population averages).

FIGS. 11A and 11B depict the lysosomal localization of MORTOR in additional cells. FIG. 11A shows lysosomal localization of MORTOR in MDCK cells expressing a YFP-conjugated MORTOR. The arrows indicate vesicles where MORTOR can be visualized on the double-membrane. FIG. 11B shows that untagged MORTOR colocalized with Lamp2 stain in HeLa cells. HeLa cells expressing an untagged MORTOR fused to an IRES-driven GFP were fixed and stained with endogenous antibodies against MORTOR and Lamp2.

FIGS. 12A and 12B show that Rag B/C CA rescue and Rag A knockdown further confirmed MORTOR acts upstream of the RagGTPases. FIG. 12A shows amounts of mTOR puncta in cells expressing different Rag B/C heterodimers and treated with Scramble, MORTOR or RRAGC siRNA. HeLa cells treated with Scramble, MORTOR siRNA, RRAGC siRNAs were co-transfected with Rap2A, RagB/C WT or RagB/C CA. Cell were starved and re-stimulated with amino acids for 5 minutes before fixing and staining for endogenous mTOR. The mTOR puncta were quantified for each combination (n>500 cells for each condition are analyzed and error bars represent ±1 S.D. of the population averages for 2 wells in one representative experiment). FIG. 12B shows that RRAGA is required for MORTOR-mediated mTOR puncta formation. Quantification shows a representative experiment. Error bars are 1±S.D. of well averages for 2 wells.

FIGS. 13A-13D show that MORTOR and Rag/mTOR interaction is specific and observed in other cell types. FIG. 13A shows that amino acids stimulated MORTOR and mTOR binding in HeLa cells. HeLa cells were treated as in FIG. 6B and immunoprecipitates were probed for mTOR and Raptor (FIG. 13B). Co-immunoprecipitation in HEK293 Ts with stably-integrated FLAG tagged NPC1 did not yield any mTORC1 components or RagGTPases bound. Cells were treated as in FIG. 6B. FIG. 13C shows reverse pulldowns using GFP-tagged RagA/Cs, which confirmed RagA/C interacts with MORTOR in HEK293T cells. Cells were treated as in FIG. 6B. FIG. 13D shows that over-expressed MORTOR migrated as multiple bands in an SDS-PAGE gel.

FIG. 14 shows that LAMTOR1-MITO overexpression reduced mTORC1 translocation to lysosomes in response to amino acids. HeLa cells expressing mitochondrially-targeted LAMTOR1 reduced mTOR puncta in amino acid treated cells. Images were taken with a confocal microscope at 63× magnification.

FIGS. 15A and 15B show that knockdown of GPR137B inhibited relative proliferation of a primary human fibroblast cell line, Hs68 (FIG. 15A), and a human cervical cancer cell line, HeLa (FIG. 15B), compared to the Control siRNA treated cells. The siRNA against RagC, a direct regulator of mTORC1 translocation, was used as positive control.

### DETAILED DESCRIPTION

The practice of the present invention will employ, unless otherwise indicated, conventional methods of medicine, pharmacology, chemistry, biochemistry, immunology, molecular biology and recombinant DNA techniques, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an,” and “the” include plural referents unless the content clearly dictates otherwise. Thus, for example, reference to “an antagonist” includes a mixture of two or more antagonists, and the like.

As used herein the terms “mTOR Complex 1,” “mTORC1” and “MTORC1” refer to a complex comprising the polypeptides mammalian Target of Rapamycin (mTOR), regulatory-associated protein of mTOR (Raptor), mammalian lethal with SEC 13 protein 8 (MLST8), PRAS40, and DETPOR. The mTORC1 functions in nutrient/energy/redox sensoring and regulating protein synthesis. The mTORC1 activity is stimulated by insulin, growth factors, serum, phosphatidic acid, amino acids and oxidative stress.

As used herein, the terms “mammalian Target of Rapamycin,” “mechanistic target of Rapamycin,” “mTOR,” “Frap1” and “RAFT1” all refer to a member of the phosphatidylinositol 3-kinase-related serine/threonine kinase having an N-terminus FRAP-ATM-TRRAP (FAT) domain; a kinase domain, a PIKK-regulatory domain (PRD) and a C-terminus FAT-C-terminal (FATC) domain. mTOR is known to regulate cell growth, proliferation, motility, survival, protein synthesis and transcription. The sequence for mTOR can be found at GenBank Accession Numbers NM_004958.3 and NP_004949.1.

As used herein, the terms “MORTOR,” “mediator of amino acid signaling to mTOR,” “G protein-coupled receptor 137b,” “GPR137b,” “GPR137B,” “G protein-coupled receptor TM7SF1,” “transmembrane 7 superfamily member 1,” and “TM7SF1” all refer to a seven transmembrane spanning G protein-coupled receptor that contains a potential dileucine-based lysosomal targeting signal and four putative N-glycosylation sites. The sequence for MORTOR/GPR137b is shown in SEQ ID NO:5 of the Sequence Listing and can be found at GenBank Accession Numbers NM_003272.2 and NP_003263.1.

The term “antagonist of MORTOR” as used herein refers to any molecule (e.g., small molecule inhibitor, protein, peptide, nucleic acid, oligonucleotide, antibody, or fragment thereof) that inhibits MORTOR activity, and/or MORTOR expression, and/or MORTOR-mediated mTORC1 signaling or localization at lysosomes. Inhibition may be complete or partial (i.e., all activity, some activity, or most activity is blocked by an antagonist). For example, an antagonist may reduce the activity of MORTOR by 10%, 20%, 30%, 40%, 50%, 60%, 70%, 80%, 90%, 100%, or any amount in between as compared to native or control levels.

An antagonist of MORTOR also includes an antagonist that inhibits a MORTOR-like protein or isoform. By “MORTOR-like protein” is meant a homolog of MORTOR, such as a G protein-coupled receptor 137a (GPR137a) or G protein-coupled receptor 137c (GPR137c). In some such embodiments, the antagonist is an antagonist of GPR137a (also known in the art as “G protein-coupled receptor 137A,” “GPR137,” “GPR137a,” “transmembrane 7 superfamily member 1-like 1 protein,” and “TM7SF1L1”), the sequence of which is shown in SEQ ID NO:6 of the Sequence Listing and may also be found at GenBank Accession Numbers NM_001170726.1 and NP_001164197.1. In other such embodiments, the antagonist is an antagonist of GPR137C (also known in the art as “GPR137c,” “transmembrane 7 superfamily member 1-like 2 protein,” and “TM7SF1L2”), the sequence of which is shown in SEQ ID NO:7 of the Sequence Listing and may also be found at GenBank Accession Numbers NM_001099652.1 and NP_001093122.1.

By a “disease or condition associated with dysregulation of mTORC1” is meant any disease or condition that is associated with mTORC1 dysregulation. It is believed that certain diseases and conditions are associated with mTOR dysregulation in the context of mTORC1 signaling. See, e.g., Laplante and Sabatini, Cell 149(2): 274-93 (2012). Upregulation of mTORC1 has been associated with various diseases and conditions, including, but not limited to, cancers, metabolic diseases or conditions, neurodegenerative diseases, and aging. Metabolic diseases or conditions include, but are not limited to type 2 diabetes, insulin resistance, hyperglycemia or hyperinsulinemia. Neurodegenerative diseases or conditions include Parkinson's disease, Alzheimer's disease, Huntington disease, amyotrophic lateral sclerosis or frontotemeporal dementia.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma. These terms include, but are not limited to, skin cancer, breast cancer, prostate cancer, lung cancer, ovarian cancer, endometrial cancer, testicular cancer, colon cancer, pancreatic cancer, brain cancer, head cancer, neck cancer, oral cavity cancer, tongue cancer, throat cancer, bladder cancer, melanoma, renal-cell carcinoma, thyroid cancer, kidney cancer, colorectal cancer, liver cancer (e.g., hepatocellular carcinoma), lymphoma, or chronic myeloid leukemia.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

As used herein, the term “small interfering RNA” or “siRNA” refer to double-stranded RNA molecules, comprising a sense strand and an antisense strand, having sufficient complementarity to one another to form a duplex. Such sense and antisense strands each have a region of complementarity ranging, for example, from about 10 to about 30 contiguous nucleotides that base pair sufficiently to form a duplex or double-stranded siRNA. Such siRNAs are able to specifically interfere with the expression of a gene by triggering the RNAi machinery (e.g., RISC) of a cell to remove RNA transcripts having identical or homologous sequences to the siRNA sequence. As described herein, the sense and antisense strands of an siRNA may each consist of only complementary regions, or one or both strands may comprise additional sequences, including non-complementary sequences, such as 5′ or 3′ overhangs. An overhang may be of any length of nonhomologous residues, for example, 2, 3, 4, 5, 6, 7, 8, 9, 10, 11, 12, 13, 14, 15, 16 or more nucleotides. In addition, siRNAs may have other modifications, such as, for example, substituted or modified nucleotides or other sequences, which contribute to either the stability of the siRNA, its delivery to a cell or tissue, or its potency in triggering RNAi. It is to be understood that the terms “strand” and “oligonucleotide” may be used interchangeably in reference to the sense and antisense strands of siRNA compositions.

As used herein, the term “small hairpin RNA” or “shRNA” refers to an RNA sequence comprising a double-stranded stem region and a loop region at one end forming a hairpin loop. The double-stranded region is typically about 19 nucleotides to about 30 nucleotides in length on each side of the stem, and the loop region is typically about three to about twelve nucleotides in length. The shRNA may include 3′- or 5′-terminal single-stranded overhangs. An overhang may be of any length of nonhomologous residues, for example, 2, 3, 4, 5, 6, 7, 8, 9, 10, 11, 12, 13, 14, 15, 16 or more nucleotides. In addition, such shRNAs may have other modifications, such as, for example, substituted or modified nucleotides or other sequences, which contribute to either the stability of the shRNA, its delivery to a cell or tissue, or its potency in triggering RNAi. In some cases, the shRNA may be derived from an siRNA, the shRNA comprising the sense strand and antisense strand of the siRNA connected by a loop.

The terms “microRNA,” “miRNA,” and MiR” are interchangeable and refer to endogenous or artificial non-coding RNAs that are capable of regulating gene expression. It is believed that miRNAs function via RNA interference. When used herein in the context of inactivation, the use of the term microRNAs is intended to include also long non-coding RNAs, piRNAs, siRNAs, and the like. Endogenous (e.g., naturally occurring) miRNAs are typically expressed from RNA polymerase II promoters and are generated from a larger transcript.

The terms “piRNA” and “Piwi-interacting RNA” are interchangeable and refer to a class of small RNAs involved in gene silencing. PiRNA molecules typically are between 26 and 31 nucleotides in length.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against the MORTOR mRNA.

A “target site” or “target sequence” is the nucleic acid sequence recognized (i.e., sufficiently complementary for hybridization) by an antisense oligonucleotide or inhibitory RNA molecule.

The term “hairpin” and “stem-loop” can be used interchangeably and refer to stem-loop structures. The stem results from two sequences of nucleic acid or modified nucleic acid annealing together to generate a duplex. The loop lies between the two strands comprising the stem.

The term “loop” refers to the part of the stem-loop between the two homologous regions (the stem) that can loop around to allow base-pairing of the two homologous regions. The loop can be composed of nucleic acid (e.g., DNA or RNA) or non-nucleic acid material(s), referred to herein as nucleotide or non-nucleotide loops. A non-nucleotide loop can also be situated at the end of a nucleotide molecule with or without a stem structure.

“Administering” an expression vector, nucleic acid, antisense oligonucleotide or inhibitory RNA molecule to a cell comprises transducing, transfecting, electroporating, translocating, fusing, phagocytosing, shooting or ballistic methods, etc., i.e., any means by which a nucleic acid can be transported across a cell membrane.

The term “derived from” is used herein to identify the original source of a molecule but is not meant to limit the method by which the molecule is made which can be, for example, by chemical synthesis or recombinant means.

By “isolated” when referring to a polynucleotide, such as a mRNA, antisense nucleic acid, or inhibitory RNA, is meant that the indicated molecule is present in the substantial absence of other biological macromolecules of the same type. For example, an “isolated siRNA molecule” refers to a polynucleotide molecule, which is substantially free of other polynucleotide molecules, e.g., other siRNA molecules that do not target the same RNA nucleotide sequence; however, the molecule may include some additional bases or moieties which do not deleteriously affect the basic characteristics of the composition.

“Substantially purified” generally refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., amino alklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30: 1911-1918; Elayadi et al. (2001) Curr. Opinion Invest. Drugs 2: 558-561; Orum et al. (2001) Curr. Opinion Mol. Ther. 3: 239-243; Koshkin et al. (1998) Tetrahedron 54: 3607-3630; Obika et al. (1998) Tetrahedron Lett. 39: 5401-5404.

The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), Dronpa, mCherry, mOrange, mPlum, Venus, firefly luciferase, Renilla luciferase, NADPH, beta-galactosidase, horseradish peroxidase, glucose oxidase, alkaline phosphatase, chloramphenical acetyl transferase, urease, MRI contrast agents (e.g., gadodiamide, gadobenic acid, gadopentetic acid, gadoteridol, gadofosveset, gadoversetamide, and gadoxetic acid), and computed tomography (CT) contrast agents (e.g., Diatrizoic acid, Metrizoic acid, Iodamide, Iotalamic acid, Ioxitalamic acid, Ioglicic acid, Acetrizoic acid, Iocarmic acid, Methiodal, Diodone, Metrizamide, Iohexol, Ioxaglic acid, Iopamidol, Iopromide, Iotrolan, Ioversol, Iopentol, Iodixanol, Iomeprol, Iobitridol, Ioxilan, Iodoxamic acid, Iotroxic acid, Ioglycamic acid, Adipiodone, Iobenzamic acid, Iopanoic acid, Iocetamic acid, Sodium iopodate, Tyropanoic acid, and Calcium iopodate).

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” to an antibody or “specifically (or selectively) immunoreactive with,” when referring to an antigen, refers to a binding reaction that is determinative of the presence of the antigen in a heterogeneous population of proteins, nucleic acids, and other biologics. Thus, under designated immunoassay conditions, the specified antibodies bind to a particular antigen at least two times the background and do not substantially bind in a significant amount to other antigens present in the sample. Specific binding to an antibody under such conditions may require an antibody that is selected for its specificity for a particular antigen. For example, polyclonal antibodies raised to an antigen from specific species such as rat, mouse, or human can be selected to obtain only those polyclonal antibodies that are specifically immunoreactive with the antigen and not with other antigens, except for polymorphic variants and alleles of the antigen. This selection may be achieved by subtracting out antibodies that cross-react with antigenic molecules from other species. A variety of immunoassay formats may be used to select antibodies specifically immunoreactive with a particular antigen. For example, solid-phase ELISA immunoassays are routinely used to select antibodies specifically immunoreactive with an antigen (see, e.g., Harlow & Lane. Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Recombinant” as used herein to describe a nucleic acid molecule means a polynucleotide of genomic, RNA, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below.

“Recombinant host cells,” “host cells,” “cells,” “cell lines,” “cell cultures,” and other such terms denoting microorganisms or higher eukaryotic cell lines cultured as unicellular entities, refer to cells which can be, or have been, used as recipients for recombinant vector or other transferred DNA or RNA, and include the original progeny of the original cell which has been transfected.

“Operably linked” refers to an arrangement of elements wherein the components so described are configured so as to perform their usual function. Thus, a given promoter operably linked to a coding sequence is capable of effecting the expression of the coding sequence when the proper enzymes are present. Expression is meant to include the transcription of any one or more of transcription of an mRNA, microRNA, microRNA mimic, or microRNA antagonist from a DNA or RNA template and can further include translation of a protein from an mRNA template. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

The term “transfection” is used to refer to the uptake of foreign DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3^{rd} edition, Cold Spring Harbor Laboratories, New York, Davis et al. (1995) Basic Methods in Molecular Biology, 2^{nd} edition, McGraw-Hill, and Chu et al. (1981) Gene 13:197. Such techniques can be used to introduce one or more exogenous DNA or RNA moieties into suitable host cells. The term refers to both stable and transient uptake of the genetic material, and includes uptake of microRNA.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

The term “about,” particularly in reference to a given quantity, is meant to encompass deviations of plus or minus five percent.

An “effective amount” of an antagonist of MORTOR is an amount sufficient to effect beneficial or desired results, such as an amount that decreases levels of MORTOR mRNA or protein or MORTOR activity. Additionally, an effective amount may decrease mTORC1 signaling and/or localization at lysosomes in cells. An effective amount can be administered in one or more administrations, applications or dosages.

By “therapeutically effective dose or amount” of an antagonist of MORTOR is intended an amount that, when administered as described herein, brings about a positive therapeutic response, such as improved recovery from a disease or condition associated with dysregulation of mTORC1. In one embodiment, the disease or condition associated with dysregulation of mTORC1 is cancer and a “therapeutically effective dose or amount” of an antagonist of MORTOR is an amount that has anti-tumor activity. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The invention is based in part on the discovery that protein mediator of amino acid signaling to mTOR (MORTOR) is involved in amino acid-induced translocation of mTORC1 to lysosomes where MORTOR forms a signaling complex with mTORC1, Ragulator, and Rag GTPases, which controls protein synthesis (see Example 1). The inventors have further shown that downregulation of expression of MORTOR by RNA interference reduces cell proliferation of cancerous cells and may be useful for treating cancer. Thus, the present invention pertains generally to compositions and methods for using antagonists of MORTOR for treating diseases and conditions associated with dysregulation of mTORC1.

In order to further an understanding of the invention, a more detailed discussion is provided below regarding MORTOR and methods of using antagonists of MORTOR for treatment of diseases and conditions associated with dysregulation of mTORC1.

In one aspect, the invention provides a method for treating diseases and conditions associated with dysregulation of mTORC1 with an antagonist of MORTOR. MORTOR antagonists disclosed herein can function to interfere with mTORC1 signaling in a variety of different ways. As disclosed herein, MORTOR functions in mTORC1 activation and translocation to lysosomes. Moreover, as disclosed in Example 1, amino acids stimulate binding of MORTOR to a Rag/mTORC1 complex. MORTOR is believed to also function in mTORC1 mediated inhibition of autophagy. In certain embodiments, an antagonist interferes with the localization of mTORC1 to lysosomes in a subject. In other embodiments, an antagonist interferes with phosphorylation of ribosomal S6 in a subject. In yet other embodiments, an antagonist inhibits binding of the GPR137 protein to mTORC1. In further embodiments, an antagonist increases autophagy in a subject.

Upregulation of mTORC1 has been associated with various diseases and conditions, including, but not limited to, cancers, metabolic diseases or conditions, neurodegenerative diseases, and aging. An “effective amount” of an antagonist of MORTOR is an amount sufficient to effect beneficial or desired results, such as an amount that decreases levels of MORTOR mRNA, MORTOR protein, or MORTOR activity. Additionally, an effective amount may decrease mTORC1 signaling and localization at lysosomes in cells. Preferably, one or more symptoms of the disease or condition associated with dysregulation of mTORC1 are ameliorated or eliminated following administration of an antagonist of MORTOR, resulting in improved recovery following treatment.

Antagonists of MOTOR can include any molecule (e.g., small molecule inhibitor, protein, peptide, nucleic acid, oligonucleotide, antibody, or fragment thereof) that inhibits MORTOR activity, and/or MORTOR expression, and/or MORTOR-mediated mTORC1 signaling or localization at lysosomes. At least three isoforms of MORTOR exist, including GPR137A, GPR137B, and GPR137C. Thus, in certain embodiments one or more of the isoforms GPR137A, GPR137B, and GPR137C, or any combination thereof, are inhibited. Inhibition may be complete or partial (i.e., all activity, some activity, or most activity is blocked by an antagonist). For example, an antagonist may reduce the activity of MORTOR by 10%, 20%, 30%, 40%, 50%, 60%, 70%, 80%, 90%, 100%, or any amount in between as compared to native or control levels.

In certain embodiments, the antagonist is an amino acid inhibitor that decreases the levels or inhibits the production of one or more amino acids in the subject. In particular embodiments, such amino acid inhibitors inhibit the level of amino acids in a subject in a manner that downregulates mTORC1 signaling. In specific embodiments, the inhibitor is an amino acid synthesis inhibitor. Amino acid synthesis inhibitors include, but are not are not limited to, amino acid analogs, e.g. leucine analogs, e.g. (±) 2-amino-bicyclo-[2,2,1]-heptane-2-carboxylic acid (BCH) (Xu et al. (2001) Diabetes 50:353-360), Leucinol (Han et al. (2012) Cell 149(2):410-24), etc.; and sulfonylureas, imidazolinones, and other amino acid derivatives.

In other embodiments, the antagonist is an antibody that specifically binds to MORTOR and interferes with its interactions with other proteins (e.g., Raptor, mTOR, or Rag GTPases) to disrupt mTORC1 signaling. In certain embodiments, the antibody specifically binds to a MORTOR protein comprising a sequence selected from the group consisting of SEQ ID NOS:5-7 or a variant thereof displaying at least about 50-99% sequence identity thereto, including any percent identity within this range, such as 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto. In certain embodiments, the antagonist is an antibody that binds to one or more isoforms of MORTOR, including any one of GPR137A, GPR137B, or GPR137C, or a combination thereof.

Antibodies that specifically bind to MORTOR can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). MORTOR antigen can be used to immunize a mammal, such as a mouse, rat, rabbit, guinea pig, monkey, or human, to produce polyclonal antibodies. If desired, a MORTOR antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to MORTOR can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 31 42, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prev. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a MORTOR antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

In other embodiments, the antagonist inhibits gene expression of MORTOR. Such antagonists of MORTOR can include, but are not limited to, antisense oligonucleotides and inhibitory RNA molecules, such as miRNAs, siRNAs, shRNAs, piRNAs, and snRNAs. Various types of inhibitors for inhibiting nucleic acid function are well known in the art. See e.g., International patent application WO/2012/018881; U.S. patent application 2011/0251261; U.S. Pat. No. 6,713,457; Kole et al. (2012) Nat. Rev. Drug Discov. 11(2):125-40; Sanghvi (2011) Curr. Protoc. Nucleic Acid Chem. Chapter 4: Unit 4.1.1-22; herein incorporated by reference in their entireties.

Antagonists can be single stranded or double stranded polynucleotides and may contain one or more chemical modifications, such as, but not limited to, locked nucleic acids, peptide nucleic acids, sugar modifications, such as 2′-O-alkyl (e.g., 2′-O-methyl, 2′-O-methoxyethyl), 2′-fluoro, and 4′-thio modifications, and backbone modifications, such as one or more phosphorothioate, morpholino, or phosphonocarboxylate linkages. In addition, inhibitory RNA molecules may have a “tail” covalently attached to their 3′- and/or 5′-end, which may be used to stabilize the RNA inhibitory molecule or enhance cellular uptake. Such tails include, but are not limited to, intercalating groups, various kinds of reporter groups, and lipophilic groups attached to the 3′ or 5′ ends of the RNA molecules. In certain embodiments, the RNA inhibitory molecule is conjugated to cholesterol or acridine. See, for example, the following for descriptions of syntheses of 3′-cholesterol or 3′-acridine modified oligonucleotides: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993); herein incorporated by reference in their entireties. Additional lipophilic moieties that can be used, include, but are not limited to, oleyl, retinyl, and cholesteryl residues, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O₃-(oleoyl)lithocholic acid, 0₃-(oleoyl)cholenic acid, dimethoxytrityl, or phenoxazine. Additional compounds, and methods of use, are set out in US Patent Publication Nos. 2010/0076056, 2009/0247608 and 2009/0131360; herein incorporated by reference in their entireties.

In one embodiment, inhibition of MORTOR function may be achieved by administering antisense oligonucleotides targeting MORTOR. The antisense oligonucleotides may be ribonucleotides or deoxyribonucleotides. Preferably, the antisense oligonucleotides have at least one chemical modification. Antisense oligonucleotides may be comprised of one or more “locked nucleic acids”. “Locked nucleic acids” (LNAs) are modified ribonucleotides that contain an extra bridge between the 2′ and 4′ carbons of the ribose sugar moiety resulting in a “locked” conformation that confers enhanced thermal stability to oligonucleotides containing the LNAs. Alternatively, the antisense oligonucleotides may comprise peptide nucleic acids (PNAs), which contain a peptide-based backbone rather than a sugar-phosphate backbone. The antisense oligonucleotides may contain one or more chemical modifications, including, but are not limited to, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages (see, for example, U.S. Pat. Nos. 6,693,187 and 7,067,641, which are herein incorporated by reference in their entireties). In some embodiments, suitable antisense oligonucleotides are 2′-O-methoxyethyl “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. Antisense oligonucleotides may comprise a sequence that is at least partially complementary to a MORTOR target nucleic acid sequence, e.g., at least about 75%, 80%, 85%, 90%, 91%, 92%, 93%, 94%, 95%, 96%, 97%, 98%, or 99% complementary to the MORTOR target nucleic acid sequence. In some embodiments, the antisense oligonucleotide may be substantially complementary to the MORTOR target nucleic acid sequence, that is at least about 95%, 96%, 97%, 98%, or 99% complementary to a target nucleic acid sequence. In one embodiment, the antisense oligonucleotide comprises a sequence that is 100% complementary to the MORTOR target nucleic acid sequence.

In another embodiment, the antagonist of MORTOR is an inhibitory RNA molecule (e.g., a miRNA, a siRNA, a shRNA, a piRNA, or a snRNA) having a single-stranded or double-stranded region that is at least partially complementary to a MORTOR target mRNA sequence, e.g., about 75%, 80%, 85%, 90%, 91%, 92%, 93%, 94%, 95%, 96%, 97%, 98%, or 99% complementary to the target mRNA sequence of MORTOR. In some embodiments, the inhibitory RNA comprises a sequence that is substantially complementary to the target mRNA sequence of MORTOR, e.g., about 95%, 96%, 97%, 98%, or 99% complementary to a target polynucleotide sequence. In other embodiments, the inhibitory RNA molecule may contain a region that has 100% complementarity to the target mRNA sequence. The inhibitory molecules may target a MORTOR mRNA comprising a sequence selected from the group consisting of SEQ ID NO:1 and SEQ ID NO:2.

In certain embodiments, the MORTOR antagonist is an RNA or RNA-like molecule having a double stranded region that is at least partially identical and partially complementary to a MORTOR target mRNA sequence, such as a mRNA comprising a sequence selected from the group consisting of SEQ ID NO:1 and SEQ ID NO:2. The inhibitory RNA molecule may be a double-stranded, small interfering RNA (siRNA) or a short hairpin RNA molecule (shRNA) comprising a stem-loop structure. The double-stranded regions of the inhibitory RNA molecule may comprise sequences that are at least partially identical and partially complementary, e.g., about 75%, 80%, 85%, 90%, 91%, 92%, 93%, 94%, 95%, 96%, 97%, 98%, or 99% identical and complementary, to the target mRNA sequence. In some embodiments, the double-stranded regions of the inhibitory RNA molecule comprise sequences that are at least substantially identical and substantially complementary to the target mRNA sequence. “Substantially identical and substantially complementary” refers to sequences that are at least about 95%, 96%, 97%, 98%, or 99% identical and complementary to a target polynucleotide sequence. In other embodiments, the double-stranded regions of the inhibitory RNA molecule may contain 100% identity and complementarity to the target mRNA sequence. In certain embodiments, the antagonist of MORTOR is an siRNA or shRNA comprising a nucleotide sequence selected from the group consisting of SEQ ID NO:3 and SEQ ID NO:4.

In certain embodiments, the inhibitory RNA molecule may comprise two complementary, single-stranded RNA molecules, such as an siRNA comprising sense and antisense strands. In other embodiments, the sense RNA sequence and the antisense RNA sequence may be encoded by a single molecule, such as an shRNA comprising two complementary sequences forming a “stem” (corresponding to sense and antisense strands) covalently linked by a single-stranded “hairpin” or loop sequence. The hairpin sequence may be from about 3 to about 12 nucleotides in length, including any length in between, such as 3, 4, 5, 6, 7, 8, 9, 10, 11, or 12 nucleotides in length. The loop can be at either end of the molecule; that is, the sense strand can be either 5′ or 3′ relative to the loop. In addition, a non-complementary duplex region (approximately one to six base pairs, for example, four CG base pairs) can be placed between the targeting duplex and the loop, for example to serve as a “CG clamp” to strengthen duplex formation.

In certain embodiments, the sense RNA strand or sequence of the siRNA or shRNA is 19 to 29 nucleotides in length or any length in between, such as 19, 20, 21, 22, 23, 24, 25, 26, 27, 28, or 29 nucleotides in length. Similarly, the antisense strand or sequence of the siRNA or shRNA may be 19 to 29 nucleotides in length or any length in between, such as 19, 20, 21, 22, 23, 24, 25, 26, 27, 28, or 29 nucleotides in length. The regions of complementarity in sense and antisense strands or sequences may be the same length. Alternatively, the sense and antisense strands may further contain non-complementary sequences, such as 3′ or 5′ overhangs or other non-complementary sequences that provide different functions for the siRNA or shRNA composition that do not contribute to base-pairing between the sense and antisense strands or sequences. Overhangs may include ribonucleotides, deoxyribonucleotides, or chemically modified nucleotides that, for example, promote enhanced nuclease resistance.

In certain embodiments, an siRNA or shRNA may comprise a 3′ overhang of from 1 to about 6 nucleotides in length, such as an overhang of 1 to about 5 nucleotides in length, 1 to about 4 nucleotides in length, or 2 to 4 nucleotides in length, including any length within these ranges, such as 1, 2, 3, 4, or 5 nucleotides in length. Either one or both strands of an siRNA may comprise a 3′ overhang. If both strands of the siRNA comprise 3′ overhangs, the length of the overhangs may be the same or different for each strand. In one embodiment, the 3′ overhang present on either one or both strands of the siRNA may be 2 nucleotides in length. For example, each strand of an siRNA may comprise a 3′ overhang of dithymidylic acid (“TT”) or diuridylic acid (“UU”) or other effective dinucleotide combinations known in the art. The 3′ terminus of an shRNA can have a non-target-complementary overhang of two or more nucleotides, for example, UU or dTdT; however, the overhangs can comprise any nucleotide including chemically modified nucleotides that, for example, promote enhanced nuclease resistance. In other embodiments, siRNAs or shRNAs comprise one or zero nucleotides overhanging at the 3′ end.

In order to enhance stability of an siRNA or shRNA, 3′ overhangs may be stabilized by including purine nucleotides, such as adenosine or guanosine nucleotides. Alternatively, substitution of pyrimidine nucleotides by modified analogues, e.g., substitution of uridine nucleotides in 3′ overhangs with 2′-deoxythymidine, may be tolerated and not affect the efficiency of RNAi degradation. In particular, the absence of a 2′-hydroxyl in the 2′-deoxythymidine may significantly enhance the nuclease resistance of the 3′ overhang.

Inhibitory RNA molecules may further comprise one or more chemical modifications, such as, but not limited to, locked nucleic acids, peptide nucleic acids, sugar modifications, such as 2′-O-alkyl (e.g., 2′-O-methyl, 2′-O-methoxyethyl), 2′-fluoro, and 4′-thio modifications, and backbone modifications, such as one or more phosphorothioate, morpholino, or phosphonocarboxylate linkages. Additionally, the inhibitory RNA molecule may be conjugated to a lipophilic molecule (e.g., cholesterol or fatty acid) to facilitate cellular uptake. Although predominantly composed of ribonucleotides, siRNAs or shRNAs may also contain one or more deoxyribonucleotides in addition to ribonucleotides along the length of one or both strands or sequences to improve efficacy or stability. The 5′ end of one or both strands or sequences of an siRNA or shRNA may also contain a phosphate group.

In certain embodiments, the invention includes compositions comprising one or more antisense oligonucleotide or inhibitory RNAs (e.g. siRNAs or shRNAs). Such compositions may comprise partially purified RNA, substantially pure RNA, synthetic RNA, or recombinantly produced RNA, as well as altered RNA that differs from naturally-occurring RNA by the addition, deletion, substitution, synthesis, and/or modification of one or more nucleotides. Such modifications may include addition of non-nucleotide material, such as to the end(s) of the siRNA or to one or more internal nucleotides of the siRNA, including modifications that make the siRNA or shRNA more effective or resistant to nuclease digestion.

Alternatively, CRISPR interference (CRISPRi) using an engineered CRISPR-Cas system can be used for downregulation of MORTOR gene expression. The CRISPR (clustered regularly interspaced short palindromic repeats) locus comprises short repetitive sequences (30-40 base pairs) separated by short spacer sequences. Transcription at the CRISPR locus results in the production of small CRISPR RNAs that contain full or partial spacer sequences. Endogenous CRISPR RNA-Cas systems comprise a Cas nuclease that is guided to target sites by a complex of two small RNAs, the CRISPR RNA (crRNA), which contains a targeting sequence, and a common trans-activating CRISPR RNA (tracrRNA). The targeting sequence can be engineered to guide a Cas nuclease to a desired specific genomic sequence. In certain embodiments, CRISPRi uses a catalytically inactive Cas nuclease. Inactivation of the Cas nuclease can be accomplished by introducing point mutations at the active site. In the case of Cas9, which is commonly used, mutations are introduced at two catalytic residues (D10A and H840A). The catalytically inactive Cas nuclease is unable to cleave dsDNA but retains the ability to target DNA and block transcriptional initiation or elongation. This is accomplished by designing a crRNA with a targeting sequence that is complementary to the promoter or exonic sequences of a target mRNA. See e.g., Richter et al. (2013) Int J Mol Sci. 14(7):14518-31; Barrangou (2013) Wiley Interdiscip Rev RNA. 4(3):267-278; Jinek et al. (20132) Science 337 (6096): 816-821; Larson et al. (2013) Nature Protocols 8 (11): 2180-2196; herein incorporated by reference. In certain embodiments, the crRNA may comprise a sequence that is at least partially complementary to a MORTOR target mRNA sequence, e.g., at least about 75%, 80%, 85%, 90%, 91%, 92%, 93%, 94%, 95%, 96%, 97%, 98%, or 99% complementary to the MORTOR target mRNA sequence. In some embodiments, the crRNA may be substantially complementary to the MORTOR target mRNA sequence, that is at least about 95%, 96%, 97%, 98%, or 99% complementary to a target polynucleotide sequence. In one embodiment, the crRNA comprises a sequence that is 100% complementary to the MORTOR target mRNA sequence. In one embodiment, the MORTOR target mRNA comprises a sequence selected from the group consisting of SEQ ID NO:1 and SEQ ID NO:2.

Knockdown can be assessed by measuring levels of the MORTOR mRNA targeted by an antisense oligonucleotide, inhibitory RNA, or CRISPRi using quantitative polymerase chain reaction (qPCR) amplification or by measuring protein levels of MORTOR by western blot or enzyme-linked immunosorbent assay (ELISA). Analyzing the protein level provides an assessment of both mRNA cleavage as well as translation inhibition. Further techniques for measuring knockdown include RNA solution hybridization, nuclease protection, northern hybridization, gene expression monitoring with a microarray, antibody binding, radioimmunoassay, and fluorescence activated cell analysis.

In another embodiment, the invention includes a method of downregulating expression of MORTOR in a subject, the method comprising: administering an effective amount of a MORTOR antagonist (antisense oligonucleotide, inhibitory RNA, or CRISPRi) described herein to the subject.

In another embodiment, the invention includes a method of downregulating expression of MORTOR in a cell, the method comprising introducing an effective amount of a MORTOR antagonist (antisense oligonucleotide, inhibitory RNA, or CRISPRi) described herein into the cell.

In another aspect, the invention includes a method for selectively decreasing the amount of a MORTOR protein in a cell of a subject, the method comprising introducing an effective amount of a MORTOR antagonist (antisense oligonucleotide, inhibitory RNA, or CRISPRi) described herein into the cell of the subject.

In certain embodiments, the MORTOR antagonist (antisense oligonucleotide, inhibitory RNA, or CRISPRi Cas/crRNA) is expressed in vivo from a vector. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, an expression vector comprises a promoter “operably linked” to at least one polynucleotide encoding a MORTOR antagonist (antisense oligonucleotide, inhibitory RNA, or CRISPRi Cas/crRNA). The phrase “operably linked” or “under transcriptional control” as used herein means that the promoter is in the correct location and orientation in relation to a polynucleotide to control the initiation of transcription by RNA polymerase and expression of the polynucleotide. In one embodiment, the recombinant polynucleotide comprises a first polynucleotide sequence encoding the sense strand of an siRNA and a second polynucleotide sequence encoding the antisense strand of an siRNA. In another embodiment, the recombinant polynucleotide comprises a polynucleotide sequence encoding an shRNA, including the sense sequence, antisense sequence, and hairpin loop of the shRNA. In another embodiment, the recombinant polynucleotide comprises a polynucleotide sequence encoding a CRISPRi crRNA and/or Cas.

In certain embodiments, the nucleic acid encoding a polynucleotide of interest is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences include UTRs which include an Internal Ribosome Entry Site (IRES) present in the leader sequences of picornaviruses such as the encephalomyocarditis virus (EMCV) UTR (Jung et al. J. Virol. (1989) 63:1651-1660. Other picornavirus UTR sequences that will also find use in the present invention include the polio leader sequence and hepatitis A virus leader and the hepatitis C IRES.

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Fluorescent markers (e.g., GFP, EGFP, Dronpa, mCherry, mOrange, mPlum, Venus, YPet, phycoerythrin), or immunologic markers can also be employed. The selectable marker employed is not believed to be important, so long as it is capable of being expressed simultaneously with the nucleic acid encoding a gene product. Further examples of selectable markers are well known to one of skill in the art.

There are a number of ways in which expression vectors may be introduced into cells. In certain embodiments of the invention, the expression construct comprises a virus or engineered construct derived from a viral genome. A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides of the present invention is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules of interest include those derived from the pox family of viruses, including vaccinia virus and avian poxvirus. By way of example, vaccinia virus recombinants expressing a nucleic acid molecule of interest (e.g., encoding siRNA or shRNA) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia based infection/transfection system can be conveniently used to provide for inducible, transient expression of the polynucleotides of interest (e.g., encoding siRNA or shRNA) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau and Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Once the expression construct has been delivered into the cell the nucleic acid encoding the antisense oligonucleotide or inhibitory RNA may be positioned and expressed at different sites. In certain embodiments, the nucleic acid encoding the antisense oligonucleotide or inhibitory RNA may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty and Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding an antisense oligonucleotide or inhibitory RNA may also be transferred in a similar manner in vivo and express the antisense oligonucleotide or inhibitory RNA.

In still another embodiment, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In certain embodiments of the invention, the liposome may be complexed with a hemagglutinating virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6):3361-3364). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular MORTOR antagonist into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). Recently, a synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In a particular example, an oligonucleotide may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

The MORTOR antagonist may comprise a detectable label in order to facilitate detection of binding of the antagonist at a target site. Detectable labels suitable for use in the present invention include any composition detectable by spectroscopic, photochemical, biochemical, immunochemical, electrical, optical, or chemical means. Useful labels in the present invention include biotin or other streptavidin-binding proteins for staining with labeled streptavidin conjugate, magnetic beads (e.g., Dynabeads), fluorescent dyes (e.g., green fluorescent protein, mCherry, cerulean fluorescent protein, phycoerythrin, YPet, fluorescein, texas red, rhodamine, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. In addition, magnetic resonance imaging (MRI) contrast agents (e.g., gadodiamide, gadobenic acid, gadopentetic acid, gadoteridol, gadofosveset, gadoversetamide, gadoxetic acid), and computed tomography (CT) contrast agents (e.g., Diatrizoic acid, Metrizoic acid, Iodamide, Iotalamic acid, Ioxitalamic acid, Ioglicic acid, Acetrizoic acid, Iocarmic acid, Methiodal, Diodone, Metrizamide, Iohexol, Ioxaglic acid, Iopamidol, Iopromide, Iotrolan, Ioversol, Iopentol, Iodixanol, Iomeprol, Iobitridol, Ioxilan, Iodoxamic acid, Iotroxic acid, Ioglycamic acid, Adipiodone, Iobenzamic acid, Iopanoic acid, Iocetamic acid, Sodium iopodate, Tyropanoic acid, Calcium iopodate) are useful as labels in medical imaging. Patents teaching the use of such labels include U.S. Pat. Nos. 3,817,837; 3,850,752; 3,939,350; 3,996,345; 4,277,437; 4,275,149; 4,366,241; 5,798,092; 5,695,739; 5,733,528; and 5,888,576.

The present invention also encompasses pharmaceutical compositions comprising one or more MORTOR antagonists (e.g., amino acid synthesis inhibitors, MORTOR specific antibodies, or antisense oligonucleotides, inhibitory RNAs, CRISPRi Cas/crRNAs, or recombinant polynucleotides or vectors encoding them) and a pharmaceutically acceptable carrier. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

Colloidal dispersion systems, such as macromolecule complexes, nanocapsules, microspheres, beads, and lipid-based systems including oil-in-water emulsions, micelles, mixed micelles, and liposomes, may be used as delivery vehicles for MORTOR antagonists described herein. Commercially available fat emulsions that are suitable for delivering MORTOR antagonists to tissues, include Intralipid, Liposyn, Liposyn II, Liposyn III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO 03/093449, which are herein incorporated by reference in their entireties.

One will generally desire to employ appropriate salts and buffers to render delivery vehicles stable and allow for uptake by target cells. Buffers also will be employed when recombinant cells are introduced into a patient. Aqueous compositions of the present invention comprise an effective amount of the delivery vehicle, dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the MORTOR antagonists of the compositions.

Compositions for use in the invention will comprise a therapeutically effective amount of at least one MORTOR antagonist (e.g., amino acid synthesis inhibitor, MORTOR specific antibody, or antisense oligonucleotide, inhibitory RNA, CRISPRi Cas/crRNA, or recombinant polynucleotide or vector encoding them). By “therapeutically effective dose or amount” of an antagonist of MORTOR is intended an amount that, when administered as described herein, brings about a positive therapeutic response, such as improved recovery from a disease or condition associated with dysregulation of mTORC1. In one embodiment, the disease or condition associated with dysregulation of mTORC1 is cancer and a “therapeutically effective dose or amount” of an antagonist of MORTOR is an amount that has anti-tumor activity.

An “effective amount” of an antagonist of MORTOR (e.g., amino acid synthesis inhibitor, MORTOR specific antibody, or antisense oligonucleotide, inhibitory RNA, or CRISPRi Cas/crRNA, or recombinant polynucleotide or vector encoding them) is an amount sufficient to effect beneficial or desired results, such as an amount that downregulates expression or reduces activity of a target MORTOR mRNA or protein. For an antisense oligonucleotide, inhibitory RNA, or CRISPRi Cas/crRNA, an effective amount may reduce translation or increase degradation of the mRNA targeted by the antisense oligonucleotide, inhibitory RNA, or crRNA. For an amino acid synthesis inhibitor or MORTOR specific antibody, an effective amount may interfere with mTORC1 signaling and/or localization at lysosomes in cells. An effective amount can be administered in one or more administrations, applications or dosages. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

Once formulated, the compositions are conventionally administered parenterally, e.g., by injection subcutaneously, intraperitoneally, intramuscularly, intralesionally, intra-arterially, or intravenously. In one embodiment, the disease being treated is cancer and compositions are administered locally into a tumor or cancerous cells. Additional formulations suitable for other modes of administration include oral and pulmonary formulations, suppositories, and transdermal formulations, aerosol, intranasal, and sustained release formulations.

Dosage treatment may be a single dose schedule or a multiple dose schedule. The exact amount necessary will vary depending on the desired response; the subject being treated; the age and general condition of the individual to be treated; the severity of the condition being treated; the mode of administration, among other factors. An appropriate effective amount can be readily determined by one of skill in the art. A “therapeutically effective amount” will fall in a relatively broad range that can be determined through routine trials using in vitro and in vivo models known in the art.

The pharmaceutical forms suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Any of the compositions described herein may be included in a kit. For example, at least one MORTOR antagonist (e.g., amino acid synthesis inhibitor, MORTOR specific antibody, or antisense oligonucleotide, inhibitory RNA, or CRISPRi Cas/crRNA, or recombinant polynucleotide or vector encoding them) may be included in a kit. The kit may also include one or more transfection reagents to facilitate delivery of polynucleotides to cells.

The components of the kit may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the MORTOR antagonist, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

Such kits may also include components that preserve or maintain the MORTOR antagonist (e.g., amino acid synthesis inhibitor, MORTOR specific antibody, or antisense oligonucleotide, inhibitory RNA, or CRISPRi Cas/crRNA, or recombinant polynucleotide or vector encoding them) or that protect against their degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented. A kit may also include utensils or devices for administering the MORTOR antagonist (e.g., amino acid synthesis inhibitor, MORTOR specific antibody, or antisense oligonucleotide, inhibitory RNA, or CRISPRi Cas/crRNA, or recombinant polynucleotide or vector encoding them) by various administration routes, such as parenteral or catheter administration or coated stent.

In another aspect, the invention includes a method of screening an agent for the ability to treat an individual having a disease or condition associated with dysregulation of mammalian target of rapamycin complex 1 (mTORC1), the method comprising: a) treating a cell expressing a MORTOR protein and mTOR with the agent; and b) measuring MORTOR protein activity in the cell, wherein a decrease in MORTOR protein activity in the cell treated with the agent compared to a cell untreated with the agent indicates that the agent will treat an individual having a disease or condition associated with dysregulation of mTORC1. The agent may be a small molecule, a nucleic acid, an antibody, or a polypeptide. The agent may affect the activity of one or more isoforms of MORTOR, including GPR137a, GPR137b, or GPR137c, or any combination thereof.

In one embodiment, the activity of MORTOR is measured by determining the amount of mTORC1 localization to lysosomes in the cell, wherein a decrease in mTORC1 localization to lysosomes in the cell treated with the agent compared to a cell untreated with the agent indicates that the agent will treat an individual having a disease or condition associated with dysregulation of mTORC1.

In another embodiment, the activity of MORTOR is measured by determining the amount of phosphorylation of ribosomal S6 in the cell, wherein a decrease in the amount of phosphorylation of ribosomal S6 in the cell treated with the agent compared to a cell untreated with the agent indicates that the agent will treat an individual having a disease or condition associated with dysregulation of mTORC1.

In another embodiment, the activity of MORTOR is measured by determining the amount of inhibition of authophagy in the cell, wherein an increase in the amount of autophagy in the cell treated with the agent compared to a cell untreated with the agent indicates that the agent will treat an individual having a disease or condition associated with dysregulation of mTORC1.

In another embodiment, the agent is screened for the ability to treat an individual having cancer, wherein the cell is a cancer cell, and the MORTOR protein activity is measured by assessing cell growth and proliferation.

In another embodiment, the agent is screened for the ability to treat an individual having a neurodegenerative disease, wherein the cell is a neuron, and the MORTOR protein activity is measured by assessing cell death.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### A Lysosomal Seven Transmembrane Protein Mediates Amino Acid Signaling to mTORC1

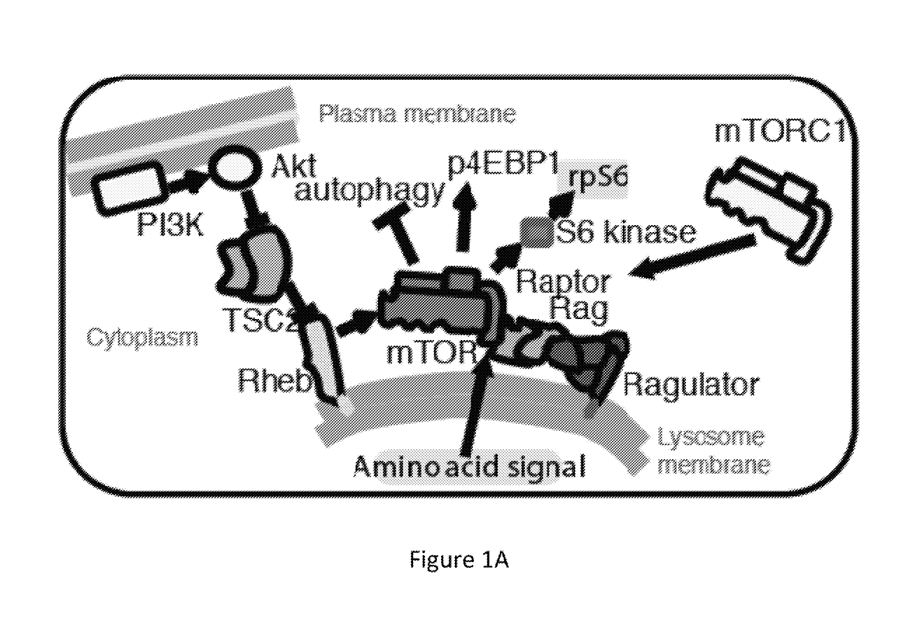

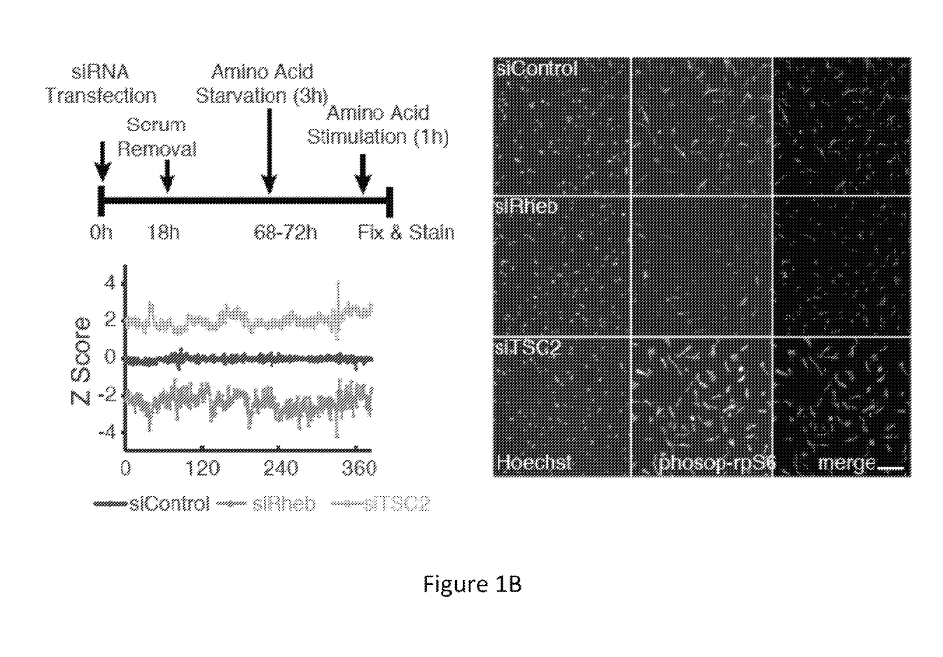

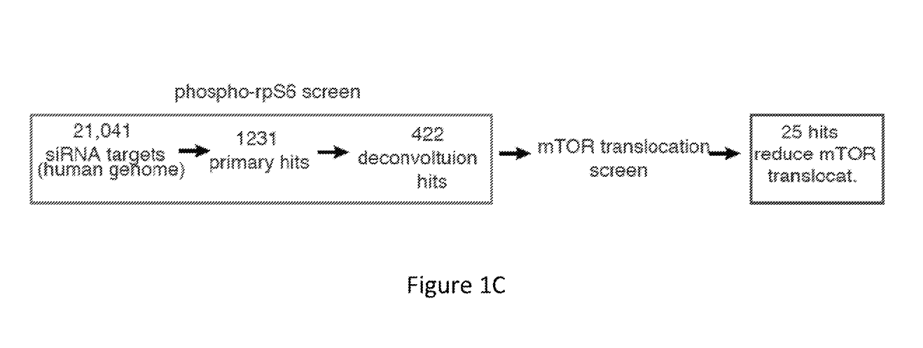

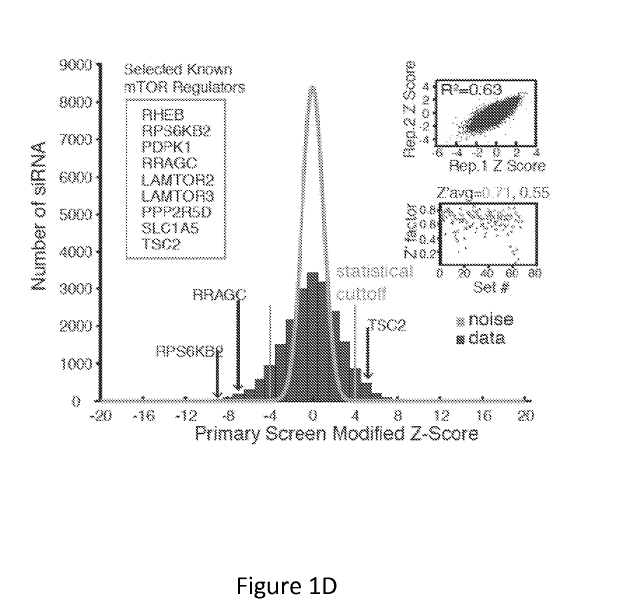

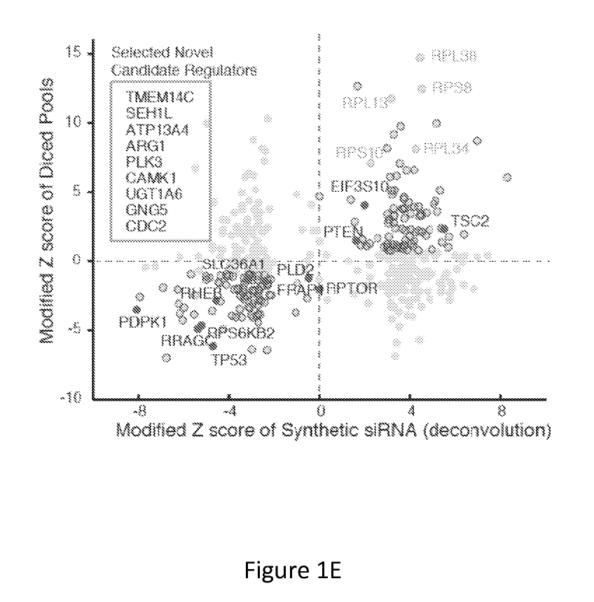

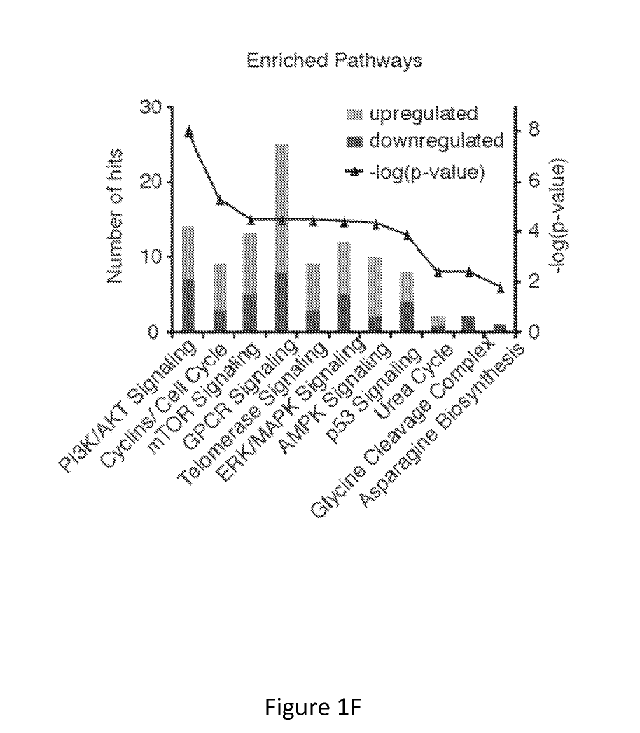

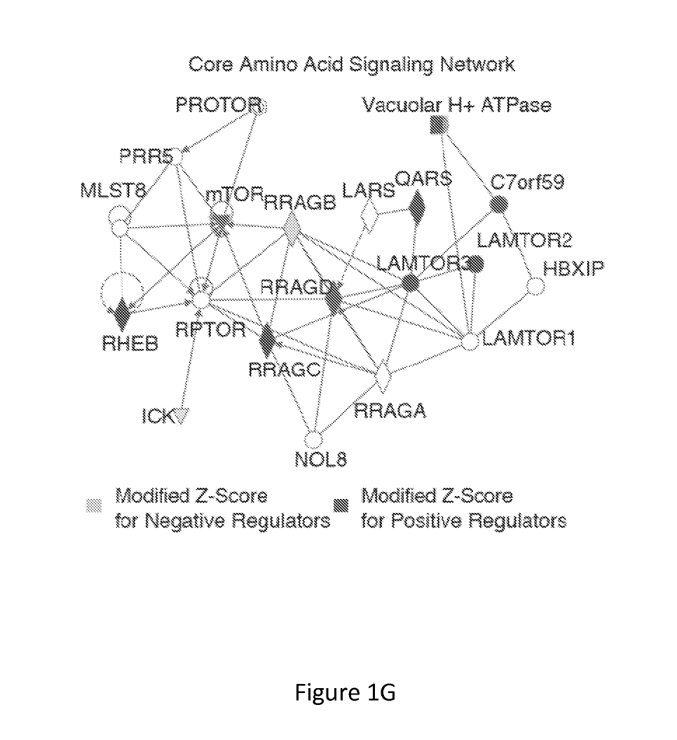

Mammalian TORC1 is an evolutionarily conserved signaling complex that includes the protein kinase mTOR and an adaptor protein raptor (FIG. 1A). It controls cell growth by integrating multiple upstream signals including nutrients, growth factors, oxygen, and stress (Inoki, K., Li, et al. (2002) “TSC2 is phosphorylated and inhibited by Akt and suppresses mTOR signaling.” Nat. Cell Biol. 4:648-657, Ma, L., et al. (2005) “Phosphorylation and functional inactivation of TSC2 by Erk implications for tuberous sclerosis and cancer pathogenesis.” Cell, 121:179-193, Roux, P. P., et al. (2004) “Tumor-promoting phorbol esters and activated Ras inactivate the tuberous sclerosis tumor suppressor complex via p90 ribosomal S6 kinase.” Proc. Natl. Acad. Sci. USA, 101:13489-13494, Inoki, K., et al. (2003) “TSC2 mediates cellular energy response to control cell growth and survival.” Cell, 115:577-590, Gwinn, D. M., et al. (2008) “AMPK phosphorylation of raptor mediates a metabolic checkpoint.” Mol. Cell, 30:214-226, Liu, L., et al. (2006) “Hypoxia-induced energy stress regulates mRNA translation and cell growth.” Mol. Cell, 21:521-531, Brugarolas, J., et al. (2004) “Regulation of mTOR function in response to hypoxia by REDD1 and the TSC1/TSC2 tumor suppressor complex.” Genes Dev., 18:2893-2904, Feng, Z., et al. (2005) “The coordinate regulation of the p53 and mTOR pathways in cells.” Proc. Natl. Acad. Sci. USA, 102:8204-8209, Lee, D. F., et al. (2007) “IKK beta suppression of TSC1 links inflammation and tumor angiogenesis via the mTOR pathway.” Cell, 130:440-455). The complex is often described as a master regulator of cell growth because it induces important anabolic processes such as ribosome biogenesis, translation, lipid biosynthesis and transcription (Ma, X. M. et al. (2009) “Molecular mechanisms of mTOR-mediated translational control.” Nat. Rev. Mol. Cell Biol., 10:307-318; Porstmann, T., et al. (2008) “SREBP activity is regulated by mTORC1 and contributes to Akt-dependent cell growth.” Cell Metab., 8:224-236; Huffman, T. A., et al. (2002) “Insulin-stimulated phosphorylation of lipin mediated by the mammalian target of rapamycin.” Proc. Natl. Acad. Sci. USA, 99:1047-1052; Chen, C., et al. (2008) “TSC-mTOR maintains quiescence and function of hematopoietic stem cells by repressing mitochondrial biogenesis and reactive oxygen species.” J. Exp. Med. 205:2397-2408; Cunningham, J. T., et al. (2007) “mTOR controls mitochondrial oxidative function through a YY1-PGC-1alpha transcriptional complex.” Nature 450:736-740). Mammalian TORC1 also suppresses autophagy, a process that breaks down and recycles cell mass when nutrients become limited (Chan, E. Y. (2009) “MTORC1 Phosphorylates the ULK1-mAtg13-FIP200 Autophagy Regulatory Complex,” Science Signaling, 2(84):pe51 (doi: 10.1126)). A diverse number of proteins that control mTORC1 are potential drug targets due to the central role of mTOR not only in metabolic dysfunction but also in cancer, diabetes, aging and neurodegenerative diseases (Zoncu, R., et al. (2011) “mTOR: from Growth Signal Integration to Cancer, Diabetes and Ageing,” Nature Reviews Molecular Cell Biology, 12:21-35; Zoncu, R., et al. (2011) “MTORC1 Senses Lysosomal Amino Acids Through an Inside-Out Mechanism that Requires the Vacuolar H(+)-ATPase,” Science, 334:678-683). Many of the growth signals upstream of mTORC1 impinge on the well-characterized PI3K/Akt/TSC2/Rheb axis of signaling (reviewed by Laplante M, et al. (2009) “mTOR signaling at a glance.” J Cell Sci., 122(Pt 20):3589-94). However, sensing of amino acids is a distinctly fundamental aspect of mTORC1 regulation that is less well understood.

Recent studies showed that mTORC1 translocates to lysosomes when amino acid levels increase and persists at lysosomes until amino acid levels fall again (Sancak, Y., et al. (2008) “The Rag GTPases Bind Raptor and Mediate Amino Acid Signaling to MTORC1,” Science, 320:1496-1501). This translocation requires pentameric Ragulator (Sancak, Y., et al. (2010) “Ragulator-Rag Complex Targets MTORC1 to Lysosomal Surface and is Necessary for its Activation by Amino Acids,” Cell, 141:290-303, Bar-Peled, L., et al. (2012) “Ragulator is a GEF for the Rag GTPases that Signal Amino Acid Levels to MTORC1,” Cell 150:1196-1208) and heterodimeric Rag GTPase; a RagA or B molecule paired with a Rag C or D molecule (Sancak, Y., et al. (2008) “The Rag GTPases Bind Raptor and Mediate Amino Acid Signaling to MTORC1,” Science, 320:1496-1501). The combined Ragulator/Rag complex is believed to associate with lysosome membranes through N-terminal fatty acyl modification of Ragulator protein Lamtor1 (Sancak, Y., et al. (2010) “Ragulator-Rag Complex Targets MTORC1 to Lysosomal Surface and is Necessary for its Activation by Amino Acids,” Cell, 141:290-303). Previous studies concluded that amino acids must be sensed in the lumen of lysosomes (Zoncu, R., et al. (2011) “mTOR: from Growth Signal Integration to Cancer, Diabetes and Ageing,” Nature Reviews Molecular Cell Biology, 12:21-35; Zoncu, R., et al. (2011) “MTORC1 Senses Lysosomal Amino Acids Through an Inside-Out Mechanism that Requires the Vacuolar H(+)-ATPase,” Science, 334:678-683), initiating a signaling pathway that ends with the recruitment of mTORC1 to lysosomes. This recruitment of mTORC1 is regulated by the GTP loading of RagA which is likely mediated by a GDP to GTP exchange activity of the Ragulator complex (Bar-Peled, L., et al. (2012) “Ragulator is a GEF for the Rag GTPases that Signal Amino Acid Levels to MTORC1,” Cell 150:1196-1208). In the GTP-bound state, RagA has an increased binding affinity for the mTORC1 component Raptor and thereby recruits the Raptor-associated mTOR protein kinase to lysosomes. Only after mTOR is localized to lysosomes can mTOR be activated by the PI3K/Akt/TSC2/Rheb signaling pathway (Sancak, Y., et al. (2008) “The Rag GTPases Bind Raptor and Mediate Amino Acid Signaling to MTORC1,” Science, 320:1496-1501). A distinct co-regulatory amino acid sensing mechanism has also been shown to exist that senses uncharged tRNA and specifically regulates the Rag D isoform (Han, J. M., et al. (2012) “Leucyl-tRNA Synthetase is an Intracellular Leucine Sensor for the MTORC1-Signaling Pathway,” Cell, 149:410-424).

Since Ragulator/Rag/mTORC1 components can interact with the H+ transporting V-ATPase responsible for acidifying secretory vesicles, Golgi, endosomes and lysosomes (Zoncu, R., et al. (2011) “mTOR: from Growth Signal Integration to Cancer, Diabetes and Ageing,” Nature Reviews Molecular Cell Biology, 12:21-35; Zoncu, R., et al. (2011) “MTORC1 Senses Lysosomal Amino Acids Through an Inside-Out Mechanism that Requires the Vacuolar H(+)-ATPase,” Science, 334:678-683), it has been suggested that it may have a secondary role in transmitting amino acid signals across the lysosomal membrane (Zoncu, R., et al. (2011) “mTOR: from Growth Signal Integration to Cancer, Diabetes and Ageing,” Nature Reviews Molecular Cell Biology, 12:21-35; Zoncu, R., et al. (2011) “MTORC1 Senses Lysosomal Amino Acids Through an Inside-Out Mechanism that Requires the Vacuolar H(+)-ATPase,” Science, 334:678-683; Cang C, et al., (2013) “mTOR Regulates Lysosomal ATP-Sensitive Two-Pore Na(+) Channels to Adapt to Metabolic State.” Cell, 152(4):778-90). It is a candidate since none of the other identified Ragulator, Rag or mTORC1 associated proteins has transmembrane spanning domains. Given that this protein already has a distinct evolutionary conserved function, we considered that an as yet unknown membrane spanning protein may exist that transmits amino acid signal changes from the lumen of the lysosomes to the cytoplasmic side. Furthermore, Raptor binding affinity to mTOR is reduced rather than enhanced upon amino acid stimulation (Kim, D. H., et al., (2002) “mTOR Interacts with Raptor to Form a Nutrient-Sensitive Complex that Signals to the Cell Growth Machinery,” Cell, 110:163-175), as is the binding between Ragulator and Rag A (Bar-Peled, L., et al. (2012) “Ragulator is a GEF for the Rag GTPases that Signal Amino Acid Levels to MTORC1,” Cell 150:1196-1208); and yet mTORC1 localizes stably to lysosomes in the presence of amino acids. These observations suggest that an unknown lysosomal scaffold protein had to exist that enhances the RagA and mTOR interaction and lysosome retention, thereby promoting persistent lysosomal localization of mTORC1 following amino acid stimulation. Thus, while much has been learned about Rag and mTORC1, the identity of the amino acid sensor in the lumen of lysosomes, the signaling steps that lead to the GTP loading of RagA and the mechanism for stable retention of active mTORC1 at lysosomes remain unknown.

Here we show results from a genome-wide siRNA screen for identifying novel regulators of the human amino acid sensing pathway. Our unbiased approach led to the discovery of a number of candidate signaling proteins in the mTORC1 signaling pathway including the protein MORTOR (Mediator of amino acid signaling to mTOR; previously named GPR137b or TM7SF1). MORTOR is a lysosomal localized transmembrane protein of previously unknown function (Spangenberg, C., et al. (1998) “Cloning and Characterization of a Novel Gene (TM7SF1) Encoding a Putative Seven-Pass Transmembrane Protein that is Upregulated During Kidney Development,” Genomics, 48:178-185; Gao, J., et al. (2012) “TM7SF1 (GPR137B): a Novel Lysosome Integral Membrane Protein,” Journal of Molecular Biology, 39:8883-8889). Intriguingly, MORTOR has seven predicted transmembrane spanning domains and a distant homology to G-protein coupled receptors. MORTOR shares evolutionary roots with the five Ragulator proteins, which were first observed in Dictyostelium discoideum. We show that MORTOR is needed for effective amino acid-induced mTORC1 translocation to lysosomes and downstream target activation. MORTOR supports the recruitment of mTORC1 by interacting with mTOR, raptor and Rag A in an amino-acid dependent manner. Furthermore, overexpression of MORTOR caused increased mTORC1 translocation and activity in response to amino acids, and rendered cells largely insensitive to amino acids. In addition, we show that Rag GTPases are required for MORTOR to mediate mTORC1 translocation, placing MORTOR at the same time upstream and downstream of Rag GTPases. Together, our data indicate that the lysosomal membrane spanning protein MORTOR is an evolutionary conserved amino acid signaling mediator that induces and maintains a lysosomal amino acid signaling complex consisting of MORTOR, mTORC1 and Rag GTPases.

Materials and Methods

Genome siRNA Screening Protocols and Analysis Methods

An siARRAY whole human genome siRNA library from ThermoFisher Scientific (Formerly Dharmacon, Cat#G-005000-025), containing 21,122 genes in 267×96-well plates, 80 siRNA pools/plate (384 well format) were screened. Three replicates of each mother siRNA plate were screened at 10 nm concentration in the primary screen in a total of 204 384-well plates. All transfections were done at HTBC using a Velocity11 Vprep with a 96 tip disposable tip head, and Lipofectamine 2000 addition. Cell additions were done with the Matrix Wellmate. All washing steps, including serum-starvation and amino acid starvation were done on the plate washer. Primary fibroblasts Hs68 cells were reverse-transfected and serum-deprived eighteen to twenty-four hours later. About 68 to 72 hours post transfection, cells were amino acid starved for 3 hours and re-stimulated with an amino acid mixture (1×AA) with similar concentrations in culture media. Cells were fixed and stained with an antibody against rpS6 phosphorylated at 240/244. Phosphorylation of rpS6 was imaged with an automated microscope (Axon, Molecular Devices), and integrated fluorescence intensity was quantified using CellProfiler software. All raw values were region-corrected with the twenty-two nearest neighboring wells to eliminate regional effects.

Primary Screen Hit Selection

Statistical hits were first determined. We first normalized the region-corrected primary screen values by dividing the averages of the standard deviations among triplicates for all triplicates on one plate. The assumption was that all siRNAs, whether or not they have an effect, have the same experimental noise. The normalized data distribution was plotted over the noise distribution with the mean=0 and sigma=1. We selected a cutoff of 4 S.D from the mean, where 0 was set such that the probability that an observed value was from the noise distribution was <0.2%. Statistical hits signify siRNAs whose effects were significantly above the experimental noise and will likely be repeated in an independent experiment. Biological hits were determined next from the statistical hits. We chose the strongest 881 hits normalized to the positive controls on each plate. Then we compiled literature hits from function/domain/keywords searches and added 350 that overlapped with the next 2000 strongest hits. Together 1231 hits were selected for deconvolution.

Deconvolution

1,231 siRNAs (4924 duplexes) were tested in duplicates at 10 nm. Scramble negative control was put in the fourth quadrant of all plates and the average of the negative controls in a 22-well neighborhood was used as a ‘zero’ point to correct for regional effects. Region corrected values were normalized by the standard deviations of all the negative controls on the plate.

Diced Libraries

The in house Meyer lab human diced library was used to screen the same assay in parallel. We also synthesized pooled siRNAs against deconvolution and human diced hits with Giardia dicer. Pools of siRNA were generated with ˜500 bp PCR products that where in vitro transcribed, diced and purified according to Liou, J., et al. (2005) “STIM is a Ca2+Sensor Essential for Ca2+-Store-Depletion-Triggered Ca2+Influx,” Current Biology, 15:1235-1241 and modified by Byoung-Ouk Park.

Bioinformatics Analysis

A combination of DAVID bioinformatics and Ingenuity pathway analysis and network was used. Hits were uploaded into DAVID bioinformatics and Ingenuity pathway analysis and the most enriched functions/canonical pathways/diseases were identified. Fisher's exact test was used to calculate a p-value. Hits within an enriched group identified from above were uploaded into Ingenuity as focus genes and a network around them was calculated using information contained in the Ingenuity Pathway Knowledge Base. Once defined, primary screen values were overlaid the networks to see which genes in the network scored significantly in the score. Some networks were manually curated to incorporate recent findings.

Cell Culture and Reagents

HS68 primary fibroblasts and HeLa cells were cultured in 10% FBS/DMEM/PSG (Gibco) at 10% CO₂ and HEK293T, MDCK and CHO cells at 5% CO₂. Lipofectamine 2000, 50× Essential amino acids, 100× non-essential amino acids and Alexa secondary antibodies were obtained from Invitrogen. BSA and cycloheximide were from Sigma and antibodies against pS6 240/244, mTOR, RagA, RagC, Raptor, p18 were from Cell Signaling. GPR137B, Lamp2, Lamp1 antibodies were from Abcam. Antibody to the HA tag (western) from Santa Cruz Biotechnology; mouse and rabbit antibodies to FLAG epitope from Sigma-Aldrich; HRP-labeled anti-mouse and anti-rabbit secondary antibodies from Jackson ImmunoResearch; antibodies to mTOR, Rictor, Raptor, Rag A, Rheb, p18, p14, beta-actin and beta-tubulin from Cell Signaling Technology; antibodies to Lamp 2, LRS and GFP (IP and western) from Abcam; anti-p62 from BD Transduction Laboratories; anti-ATP6V1A from GeneTex. Transfection reagents GeneSilencer (Gelantis) was used for co-transfection of siRNA and plasmids in HeLas, Fugene 6, and HPExtreme DNA (Roche) for plasmid transfection into HeLas and HEK293 Ts, and Lipofectamine 2000 (Invitrogen) for DNA or siRNA transfections into HS68s, HEK293 Ts, MDCKs and CHO cells. HA-tagged RagA constructs and LC3-GFP were obtained from Addgene. Plasmids GPR137b-3×FLAG in pBMN vector. HA-GST-RagA in pRK5 and GFP-RagC in pEGFP (Clonetech).

Imaging

Cells were fixed in 4% paraformaldehyde for 30 minutes at RT, followed by 15 minutes of 0.2% Triton permeabilization on ice. 3% BSA was used for blocking at 30 minutes at RT. Primary antibody was added from 1/50 to 1/500 overnight at 4 C. Hoechst and secondary antibody were added post washing for 1 to 2 hours at room temperature. Images were taken either with 20× objective on an automated inverted epifluorescent microscope (Axon from molecular devices) or with a 60× oil objective on a confocal microscope (Leica, Nikon). Mouse kidney tissue sections were prepared by fixing overnight in 4% paraformaldehyde post dissection and cut into 50 μm slices using a Vibratome.

The mTOR translocation images were acquired with a 20× objective on an automated inverted epifluorescent microscope (Axon from molecular devices). A matlab script that picked out peak intensities after background subtraction and Gaussian filtering was used to segment the mTOR puncta in an expanded ring around the nucleus (perinuclear region) in each cell. If co-staining with Lamp2, punta was segmented in the Lamp2 channel. A correlation coefficient for all the puncta intensities at the same location for mTOR and Lamp2 was measured for each cell and averaged for the entire well. If Lamp2 staining was absent, then the puncta intensity ratioed over the mean intensity in the ring in the mTOR channel was used to represent the fraction of mTOR staining found inside the puncta in the perinuclear region. When necessary, correlation coefficients or puncta intensity ratio were normalized to control-treated cells for comparing across independent experiments.

Cell Lysis and Immunoprecipitation

One day before transfection, 2,000,000 HEK293T cells were plated in 10 cm culture dishes. The cells were transfected with 2 μg of cDNA expression vectors using Lipofectamine 2000 (Life Technologies). Four hours after transfection, the cells were washed and incubated in DMEM with 10% FBS without antibiotics for 18 hours. When amino acid sensitivity was investigated, cells were transfected for 18 or 38 hours followed by 4 hours of amino acid starvation. The cells were then stimulated with amino acid for 7 minutes. The cells were incubated in Triton X-100 lysis buffer (1% Triton X-100, 40 mM HEPES pH 7.4, 2 mM EDTA, 200 mM NaCl, phosSTOP and protease inhibitor cocktail (Roche)) for 1 hour, followed by sonication at 4° C. For in-cell crosslinking experiments, DSP was used as previously described (Sancak, Y., et al. (2008) “The Rag GTPases Bind Raptor and Mediate Amino Acid Signaling to MTORC1,” Science, 320:1496-1501) prior to cell lysis. The lysates were centrifuged at 20,000×g for 30 minutes. For immunoprecipitation, primary antibodies were cross-linked to Protein G beads (Life technologies) using DMP (Thermo scientific). For anti-FLAG-immunoprecipitation, the FLAG-M2 affinity gel (Life technologies) was used. The cleared lysates were incubated with the beads at 4° C. for 2 hours or overnight. The beads were washed five times with ice-cold lysis buffer. Immunoprecipitated proteins were denatured in the SDS sample buffer and separated by SDS-PAGE. The intensity of bands were quantified using ImageJ (NIH).

To reduce GPR137b-FLAG smear band on western blotting, 300,000 HEK293T cells were plated on 6-well dish. Twenty-hours later, pBMN-GPR137b-3×FLAG was transiently transfected using X-tremeGENE HP for 20 hours and the cells were frozen. 100 μl of 1% SDS containing buffer (1% SDS, 50 mM HEPES [pH 7.4], 5 mM DTT, protease inhibitor cocktail (Roche)) were added to the frozen cell pellet and incubated at 37° C. for 15 minutes. The lysate was briefly sonicated and centrifuged at room temperature then diluted in 1.2 mL of Triton X-100 buffer (0.75% Triton X-100, 50 mM HEPES pH 7.4, 1 mM EDTA, 200 mM NaCl, and protease inhibitor cocktail (Roche)). GPR137b-3×FLAG were immunoprecipitated from a 0.5 mg protein-equivalent amount of lysate using 20 μl of FLAG-M2 affinity gel slurry. The beads was washed five times with Triton X-100 buffer. 100 μl of urea sample buffer (3% SDS, 600 mM DTT, 8M urea, 187.5 mM Tris-HCl pH 6.8, 25% glycerol, 0.022% bromophenol and 1× Halt protease inhibitor cocktail (Thermo scientific)) was added to the beads and incubated at 37° C. for 15 minutes. 20 μl of sample were loaded to resolve the protein on 4-12% NuPAGE Bis-Tris precast gels (Life technologies).

Preparation of Cytosol and Crude Membrane Fractions

Cells were rinsed with ice-cold PBS, then swollen in ice-cold 10 mM HEPES. The cells were then suspended in SEAT buffer (Triethanolamine/Acetic Acid pH 7.6, 5% sucrose, 1 mM EDTA, protease inhibitor EDTA-free) and homogenized with a 25-gauge needle. The resultant mixture was centrifuged at 900 g for 5 minutes at 4° C. to remove nuclei. The postnuclear supernatant was centrifuged subsequently at 95,000 g for 20 minutes. The supernatant was kept as the cytosolic fraction. The pellet was incubated in membrane extraction buffer (1% Triton X-100, 40 mM HEPES pH 7.4, 2 mM EDTA, 200 mM NaCl, phosSTOP and protease inhibitor cocktail) at 4° C. for 1 hour, then sonicated. The lysate was centrifuged at 20,000 g for 30 minutes at 4° C. The supernatant was collected as the membrane fraction.

Results

Genome-Wide siRNA Screen Identifies Regulators of Amino Acid Signaling

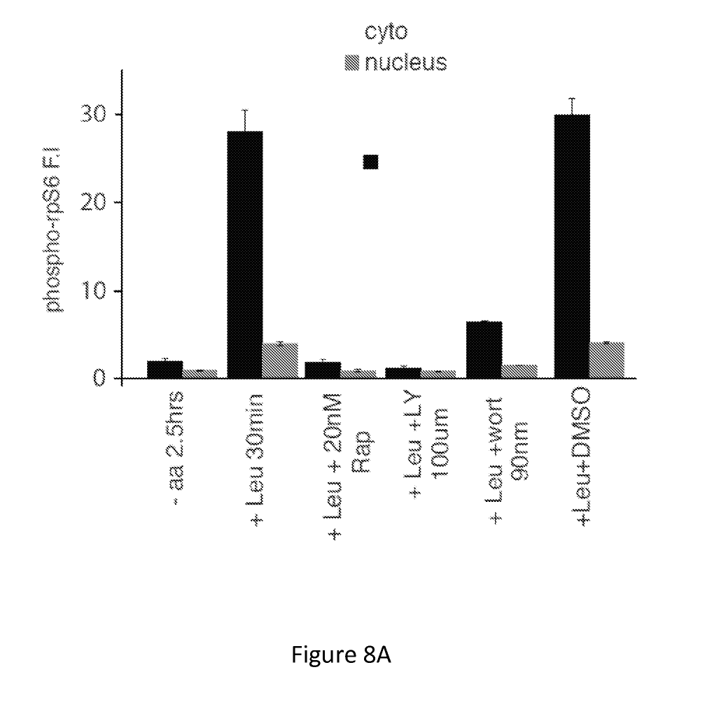

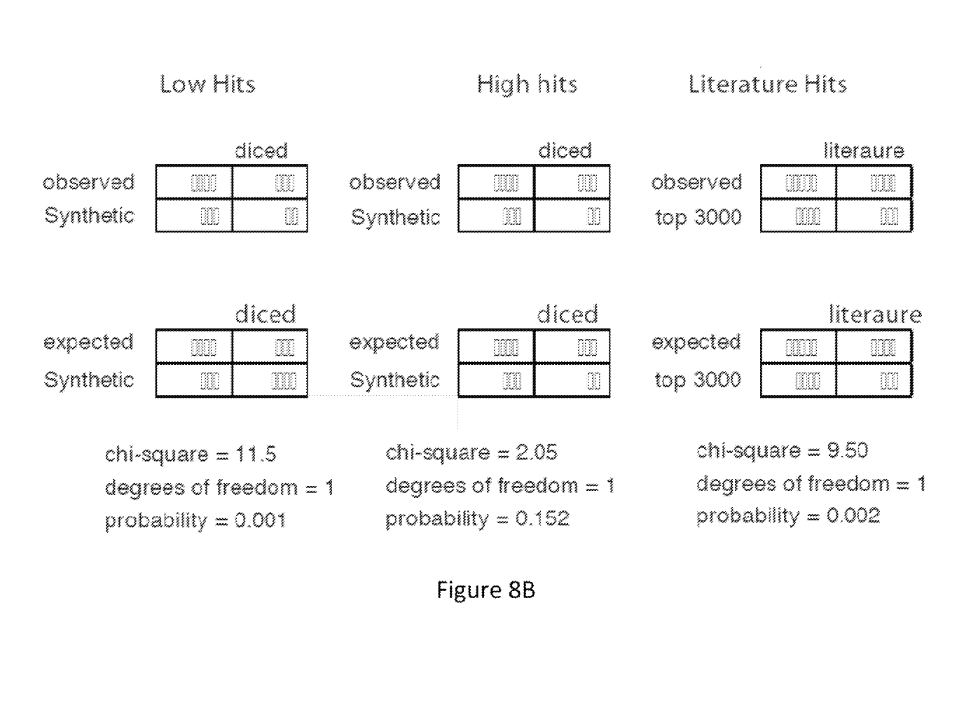

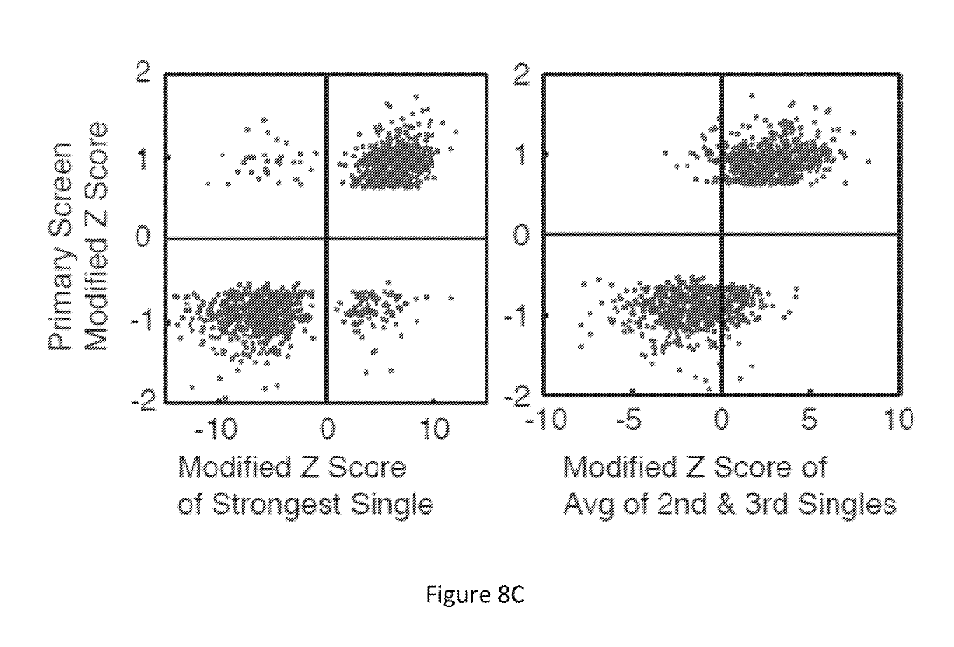

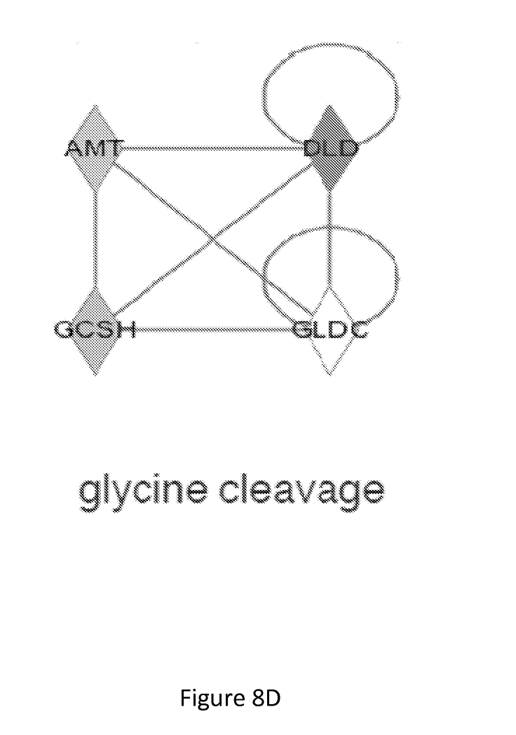

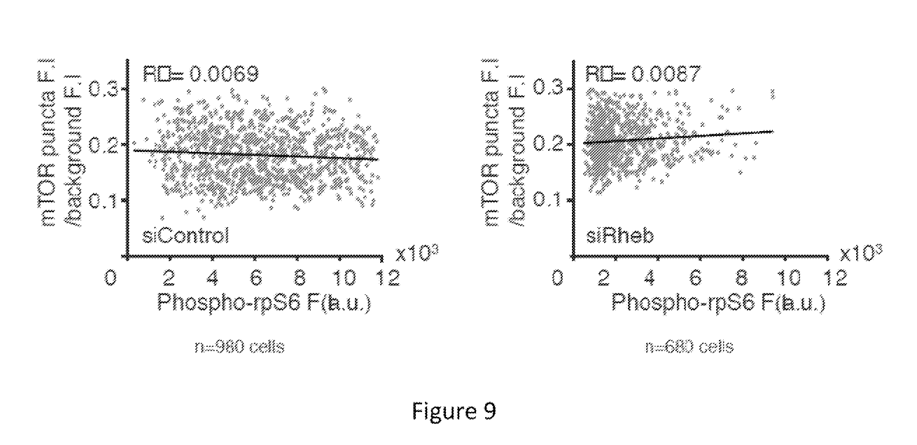

A microscopy-based assay was developed to measure amino acid-stimulated mTORC1 activation to identify regulatory proteins in the lysosomal amino acid sensing signaling pathway. Specifically, the assay monitored the amino acid-triggered increase in phosphorylation of the ribosomal S6 protein (rpS6) at residue 240/244 using a monoclonal antibody. This assay confirmed an earlier finding (Rosner, M., et al. (2010) “Evidence for Cell Cycle-Dependent, Rapamycin-Resistant Phosphorylation of Ribosomal Protein S6 at S240/244,” Amino Acids, 39:1487-1492) that rpS6 phosphorylation closely correlates with the state of mTORC1 activation (FIG. 8A). The assay was sensitive for capturing both positive and negative regulators, as knocking down the mTORC1 co-activator Rheb and the mTORC1 suppressor TSC2 caused strong opposing changes in S6 phosphorylation and was consistent across the whole screen at roughly +2 or −2 standard deviations away from the plate median (FIG. 1B). We expected that this assay would be suitable not only to discover novel regulators in the amino acid signaling pathway but also in parallel signaling pathways required for mTORC1 and S6 kinase activation (FIG. 8A).

The 384-well formatted screen made use of human primary foreskin fibroblasts (HS68 cells) since many transformed cell lines are known to have defects in cell metabolism (Cheong H, et al. (2012) “Therapeutic targets in cancer cell metabolism and autophagy.” Nat Biotechnol., 30(7):671-8). 21,2041 separate siRNA pools (each containing four siRNA duplexes per targeted human gene) were used, and each siRNA pool was analyzed in three different wells. Cells were reverse-transfected with siRNAs and serum was removed to reduce growth factor signaling to mTORC1 and also slow cell growth to minimize cell density differences. Prior to fixation, amino acids were removed for 3 hours to further reduce mTORC1 activity, followed by a 1 hour re-addition of amino acids (FIG. 1B). Fixed cells were stained with anti-rpS6 antibody and nuclear marker, images were acquired using an automated fluorescence microscope, and automated image analysis was used to measure the relative rpS6 level in each cell. Measurements of rpS6 between siRNA replicates were repeatable for most of the targeted genes (R²=0.633) and z′ factors were largely consistent over the 68 groups of plates based on the positive and negative control siRNAs (targeting Rheb and TSC2, respectively) (FIG. 1C, insets). This full genome screen was preceded by a focused pilot screen of 2300 putative signaling proteins using a previously described set of Dicer-based siRNAs targeting known and putative signaling proteins (Liou, J., et al. (2005) “STIM is a Ca2+Sensor Essential for Ca2+-Store-Depletion-Triggered Ca2+Influx,” Current Biology, 15:1235-1241). The different origins and type of siRNA pools made it possible to compare the pilot and full-genome screens. We confirmed a statistic significance of reproducible hits between the two screens when comparing overlapping gene targets (FIG. 1E and FIG. 8B).

The primary screen generated a roughly symmetric distribution of positive and negative changes in rpS6 phosphorylation mediated by different siRNAs. Using a confidence threshold based on the standard error between triplicates (see Methods), 2838 of the targeted genes had deviations from the mean larger than expected from a stochastic noise model (FIG. 1D). Further analysis was restricted to siRNA pools that caused positive or negative changes in phosphorylation close to those observed for Rheb and TSC2 knockdown. Choosing relatively high positive and negative thresholds made sense since we found that well-known regulators of mTOR pathway such as RPS6KB2, RRAGC, and PDPK1 scored strongly in the primary screen. Two subsets of siRNAs were used for more detailed analysis, a top scoring set consisting of the 881 siRNAs with the largest effects on rpS6 and a second set of 350 siRNAs that we selected subjectively from the next strongest 1100 hits based on an their domain organization and plausible enzymatic roles as well as functional insights gained from manual literature searches. FIG. 1C outlines the selection and validation protocol to identify candidate regulatory proteins.

To increase confidence that the effects of the 1231 siRNAs are not a result of off-target effects, each of the four siRNAs used in the original pooled experiment was tested for their individual effect on rpS6 phosphorylation. We noticed that the effect of the strongest single siRNA closely correlated with the result from the pooled siRNA primary screen (FIG. 8C), suggesting that the strongest siRNA typically dominates the pooled siRNA phenotype. To gain an independent validation and exclude potential off-target effects, we averaged the effect of the second and third strongest single siRNA phenotype and determined whether the combined effect is significant and matches the direction of the signal change from the primary screen and the single strongest siRNA result (FIG. 8C). By excluding pools that did not conform to this criterion, we were left with 422 higher confidence genes for which we had independent siRNA evidence that the loss of their gene products alters amino acid-induced mTORC1 activation and S6 phosphorylation.

Assigning Validated Genes to Known Signaling Pathway Modules

We confirmed that this validated set of genes is enriched for mTOR related genes by performing a pathway analysis using Ingenuity. As shown in FIG. 1F, genes associated with insulin/growth factor, GPCR, PI3K/AKT, ERK/MAPK, Hedgehog, or cell cycle signaling pathways were all significantly enriched in the validated set (FIG. 1F). This suggests a close feedback connection from signaling pathways that regulate growth and proliferation to mTORC1 signaling. In addition to these pathways, the screen also identified genes from the stress response, such as p53 and AMPK pathways. This may also explain why genes in the urea cycle and regulators of telomerases are also enriched in the screen.

We also manually constructed a network of the mTORC1 amino acid sensing module from up-to-date literatures (FIG. 1G). The primary screen captured many of the known genes involved in amino acid and mTORC1 signaling, such as Rheb and RRAGC. In addition, three of the five Ragulator components, LamTOR2, LamTOR3 and LamTOR4, as well as the D1 subunit of the V0 transmembrane domain of the v-ATPase (ATP6V0D1) scored strongly in the screen as positive regulators. Leucyl tRNA synthetase, recently shown to function as a GAP for RRAGD (Han, J. M., et al. (2012) “Leucyl-tRNA Synthetase is an Intracellular Leucine Sensor for the MTORC1-Signaling Pathway,” Cell, 149:410-424) did not show an effect in the screen. However, glutamine-tRNA synthetase, which has been shown to interact with RRAGC, had a strong phenotype (FIG. 1F). It is also interesting to note that many of the ribosomal subunits were present in the validated set and were also among the strongest hits in the primary screen (FIG. 1E). It is plausible that knocking down ribosomal components triggers a direct feedback from a reduced translation rate to mTORC1 activation. Previous studies showed that a translation block by cyclohexamide increases free amino acids levels, which in turn up-regulates mTORC1 signaling (Beugnet, A., et al. (2003) “Regulation of Targets of mTOR (Mammalian Target of Rapamycin) Signaling by Intracellular Amino Acid Availability,” Biochemical Journal, 372:555-566) “Regulation of Targets of mTOR (Mammalian Target of Rapamycin) Signaling by Intracellular Amino Acid Availability,” Biochemical Journal, 372:555-566). The same feedback mechanism may also engage in cells with knocked down ribosomal components or regulators of translation, which may lower protein synthesis, increases free amino acids and in turn induces mTORC1 activation.

Amino acid metabolism pathways, especially the glycine cleavage pathway in mitochondria and the asparagine biosynthesis pathway were also significantly over represented in the dataset. The glycine cleavage complex is comprised of 4 proteins, DLD, GCSH, AMT and GLDC (Motokawa, Y. et al. (1969) “Glycine metabolism by rat liver mitochondria. IV. Isolation and characterization of hydrogen carrier protein, an essential factor for glycine metabolism.” Arch. Biochem. Biophys., 135:402-409, Motokawa, Y. et al. (1974) “Glycine metabolism by rat liver mitochondria. Reconstitution of the reversible glycine cleavage system with partially purified protein components.” Arch. Biochem. Biophys., 164:624-633, Motokawa, Y. et al. (1974) “Glycine metabolism by rat liver mitochondria. Isolation and some properties of the protein-bound intermediate of the reversible glycine cleavage reaction.” Arch. Biochem. Biophys., 164:634-640). Three of these proteins were strong hits in the primary screen. A function of this system is to convert glycine to serine. Our study argues that the glycine cleavage pathway is rate-limiting and could potentially be targeted by drugs as a means to regulate mTORC1 signaling (FIG. 8D). Together, this network analysis shows that our screen identified many of the known regulators of mTORC1 but also identified new mTORC1 regulatory modules. This increased our confidence that the validated set might also include novel upstream regulators of lysosomal amino acid sensing.

Regulators of Amino Acid-Triggered Translocation of mTORC1 to Lysosomes

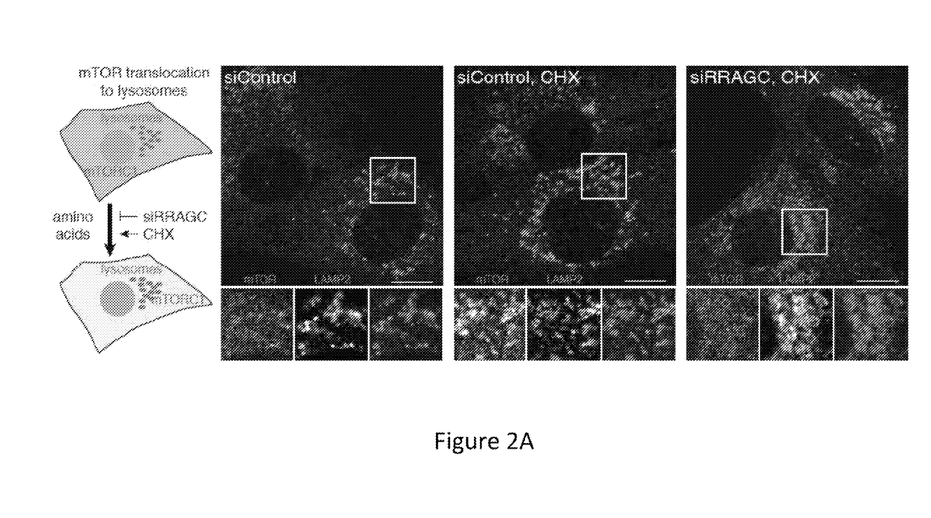

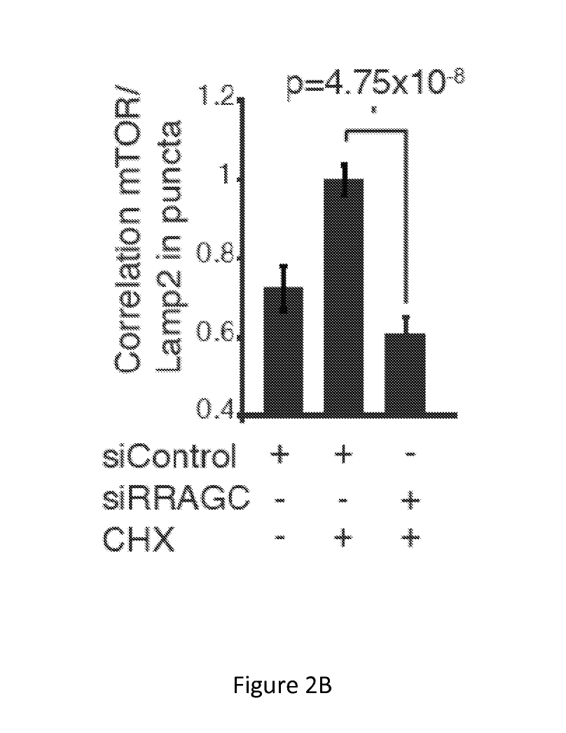

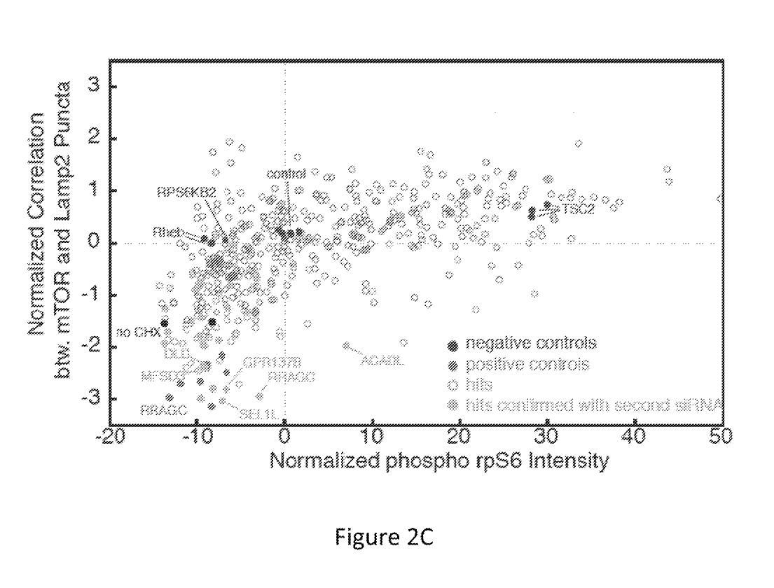

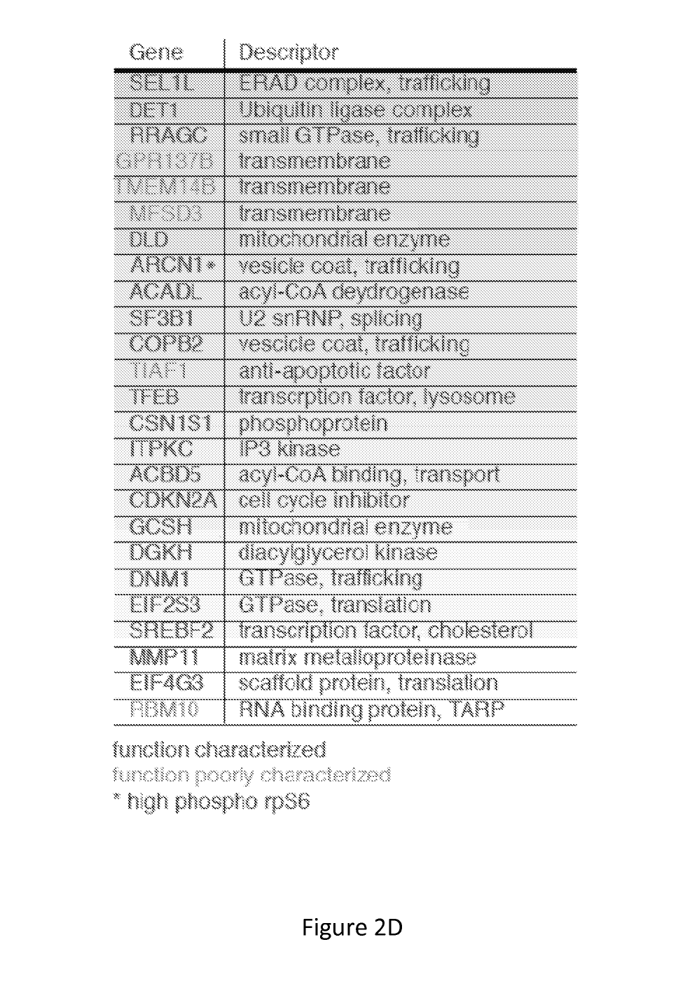

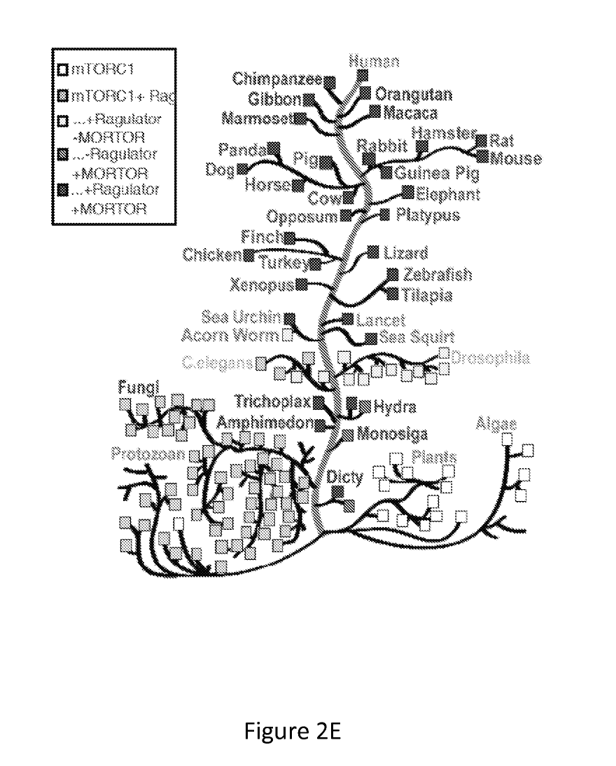

The Rag-mediated translocation of mTORC1 to lysosomes is considered to be the final step in the amino acid sensing pathway (Sancak, Y., et al. (2008) “The Rag GTPases Bind Raptor and Mediate Amino Acid Signaling to MTORC1,” Science, 320:1496-1501, Sancak, Y., et al. (2010) “Ragulator-Rag Complex Targets MTORC1 to Lysosomal Surface and is Necessary for its Activation by Amino Acids,” Cell, 141:290-303) (FIG. 2A). We determined whether a subset of the 422 candidate genes has an effect on rpS6 phosphorylation by increasing or decreasing the translocation of mTORC1 to lysosomes. We measured mTORC1 translocation in fixed cells by using automated imaging to monitor changes in the degree of translocation of mTOR from the cytosol to LAMP2-positive late endosomes/lysosomes in response to an amino acid starvation/readdition protocol. In order to avoid regulators that may act indirectly by altering amino acid uptake by cells, we rapidly increased intracellular amino acid levels by blocking protein synthesis using cycloheximide (Beugnet, A., et al. (2003) “Regulation of Targets of mTOR (Mammalian Target of Rapamycin) Signaling by Intracellular Amino Acid Availability,” Biochemical Journal, 372:555-566). Cycloheximide was added for only 5 minutes after 4 hours of amino acid starvation which was sufficient to induce maximal mTORC1 translocation (FIG. 2B). This enabled us to more specifically identify proteins involved in intracellular sensing rather than in regulating amino acid import.

Given that mTORC1 translocation is necessary for kinase activation and that rpS6 phosphorylation is a sensitive reporter of mTORC1 activity, we expected that the effect of a subset of the siRNAs on rpS6 phosphorylation would closely correlate with their effect on mTORC1 translocation. Indeed, a small subset of hits significantly reduced mTOR translocation to lysosomes while also consistently reducing rpS6 (FIG. 2C). This provided support for the previous model that translocation of mTORC1 to lysosomes is a necessary step for amino acid-induced rpS6 phosphorylation. For a larger fraction of the candidate genes, there was no correlation between lysosomal mTOR localization and rpS6 phosphorylation (FIG. 2C). These genes likely act in the PI3K/Akt/TSC2/Rheb signaling pathway that activates lysosomal localized mTOR. Alternatively, they may act downstream of mTORC1 in the regulation of S6 kinase or the regulation of S6 phosphorylation by an alternative mechanism. Here we focused on the small subset of candidate regulators whose knockdown suppressed the amino acid-triggered mTORC1 localization to lysosomes.

FIG. 2D shows a list with twenty five genes ranked according to the strength of the suppression of mTOR localization to lysosomes when we targeted each gene by siRNA. RRAGC was identified as a top hit regulating mTOR translocation, confirming the importance of Rag GTPases in recruiting mTORC1 to lysosomes. Trafficking proteins such as SEL1L, COPB and ARCN1 were strong hits as were the master regulator for lysosome biosynthesis, TFEB, the cholesterol biosynthesis transcription factor SREBP2, and two RNA binding and splicing factors RBM10 and SF3B1 that have functions that are less well understood. These genes may act indirectly on lysosomal mTORC1 localization by altering the concentration or identity of lysosomes or the concentration of key mTORC1 regulatory components. Furthermore, the glycine cleavage complex proteins DLD and GCSH did again show up but now as regulators of mTORC1 translocation to lysosome. The synthesis and transport of acyl-CoA also clearly plays a role in mTORC1 translocation as the acyl-CoA regulators ACADL and ACDB5 were both present in the small set. This suggests a potential involvement of specific amino acid metabolism pathways or other metabolic products in regulating the localization of mTORC1. There are also a number of proteins that may be indirectly linked to mTORC1 signaling such as ITPKC, a Ca2+ regulate IP(1,4,5)P3 kinase needed to build up IP5, IP6 and IP7 in cells which in turn are known to have important roles in metabolism (e.g., Chakraborty A, et al., (2010) “Inositol pyrophosphates inhibit Akt signaling, thereby regulating insulin sensitivity and weight gain.” Cell, 143(6):897-910). Particularly interesting was the identification of three transmembrane proteins MFSD3, TMEM14b and GPR137B in this set that had as of yet no known function.

An Evolutionary Conserved Membrane Protein MORTOR Regulates Amino Acid-Induced mTORC1 Translocation and Activation

One of the membrane proteins that was identified in the mTORC1 translocation screen was TM7SF1. TM7SF1 is a protein comprising seven transmembrane domains that was first reported to be upregulated during kidney development (Spangenberg, C., et al. (1998) “Cloning and Characterization of a Novel Gene (TM7SF1) Encoding a Putative Seven-Pass Transmembrane Protein that is Upregulated During Kidney Development,” Genomics, 48:178-185). The same protein was subsequently annotated as GPR137b due to its weak sequence homology to G-protein coupled receptors. A more recent proteomics and imaging study showed that GPR137b is localized to lysosome membranes (Gao, J., et al. (2012) “TM7SF1 (GPR137B): a Novel Lysosome Integral Membrane Protein,” Journal of Molecular Biology, 39:8883-8889). Nevertheless, no function for TM7SF1 or GPR137B has previously been reported. In reference to its role in amino acid-induced mTORC1 translocation, we are using below the name MORTOR instead of the place holder names TM7SF1 or GPR137B.

An evolutionary analysis of MORTOR revealed that MORTOR and all five Ragulator homologs first appeared in the slime mold Dictyostelium discoideum. We used the NCBI server to perform Blast analysis of the respective human genes and homologs from different species, which were verified using a reverse best blast criterion back to the same human gene (Collins and Meyer (2011) Evolutionary origins of STIM1 and STIM2 within ancient Ca2+ signaling systems. Trends Cell Biol. 2011 April; 21(4):202-11). MORTOR was of particular interest because the ancestral homologs of MFSD3 and TMEM14b were more distant, and the joint appearance of MORTOR with Ragulator in Dictyostelium was remarkable since only a relatively small number of human genes are known to first appear at this branch point. FIG. 2E shows a manually curated human centric analysis of 144 genomes for the joint presence or absence of mTORC1 (white squares; raptor and mTOR homologs both present) and of Rag/mTORC1 (grey squares; raptor and mTOR as well as RagA and RagC homologs present). Interestingly, plants, algae, many protozoans and a few fungi have lost their Rag's but kept raptor and mTOR homologs, and a few species have kept Rag's but lost their raptor and mTOR homologs. It was also interesting that Ragulator and MORTOR homologs were both lost in all fungi, but are again both present at the advent of animal multi-cellularity in Trichoplax adhaerens and Amphimedon queenslandica (FIG. 2E, dark gray; marks species with Rag/mTORC1, Ragulator and MORTOR). They are also consistently present in all species in the vertebrate branch. MORTOR as well as Ragulator homologs were also lost independently of each other in a number of species. For example, MORTOR homologs are missing in the sequenced insect and worm genomes (FIG. 2E) while Ragulator homologs are missing in some of the worm species as well as in Monosiga Brevicollis and Entamoeba histolytica, which nevertheless have a MORTOR homolog (FIG. 2E). It should be noted that individual Ragulator components are absent in a number of ancestral invertebrate species, suggesting that Ragulator may not exclusively function as a pentamer or that some of the subunits have alternative functions (as shown in FIG. 2E, some have at least Lamtor1 plus 2 more of the other subunits). These evolutionary considerations show that the invertebrate and early eukaryotic mTORC1 control systems can in some cases differ from the human one.

Control Experiments Validating MORTOR as an mTORC1 Regulator

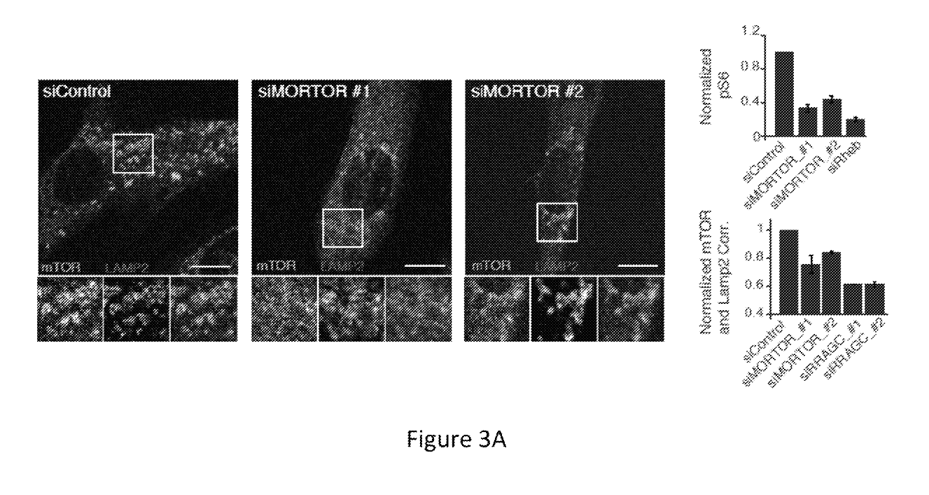

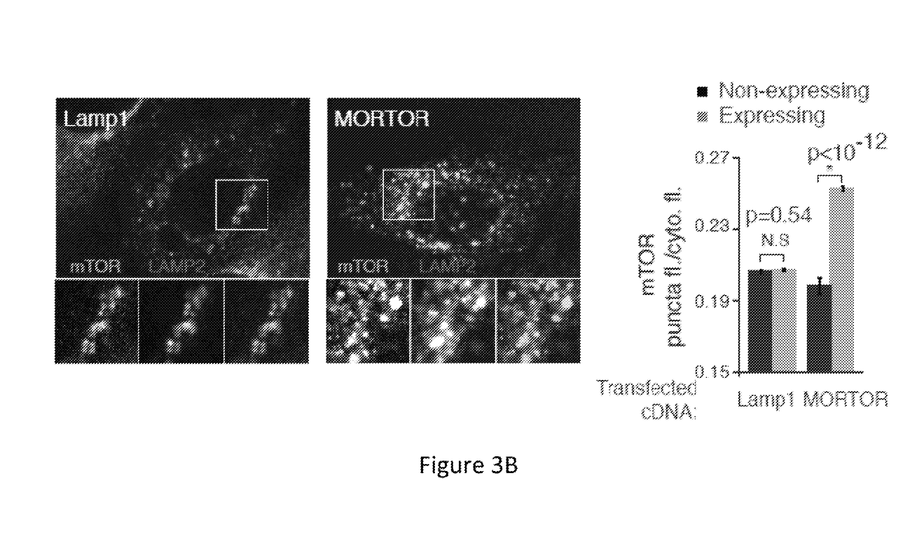

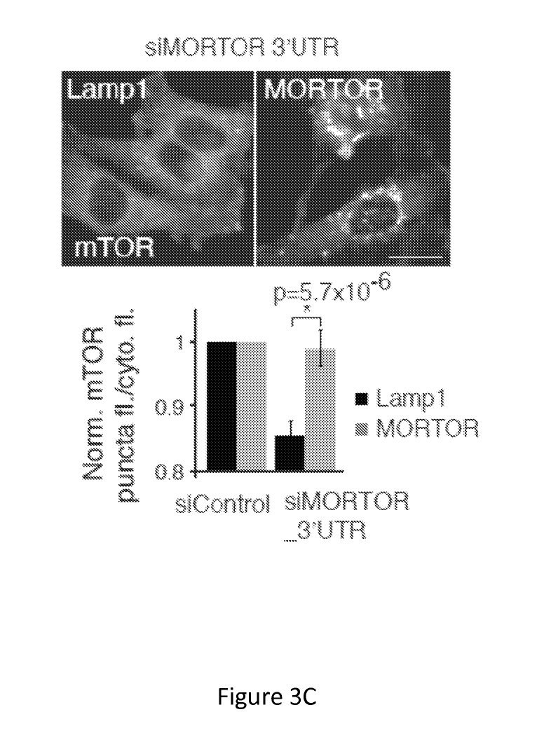

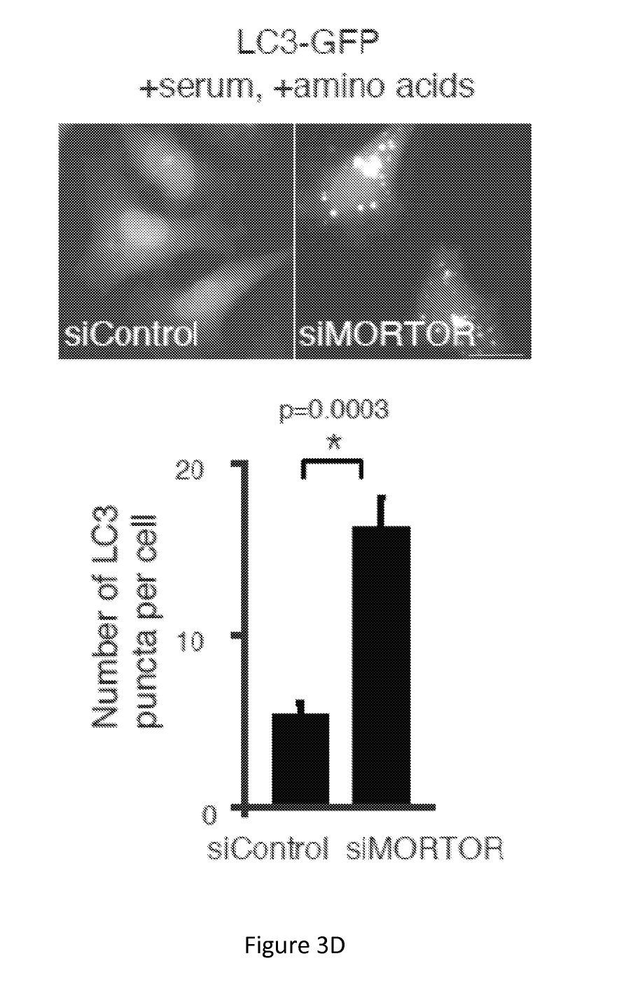

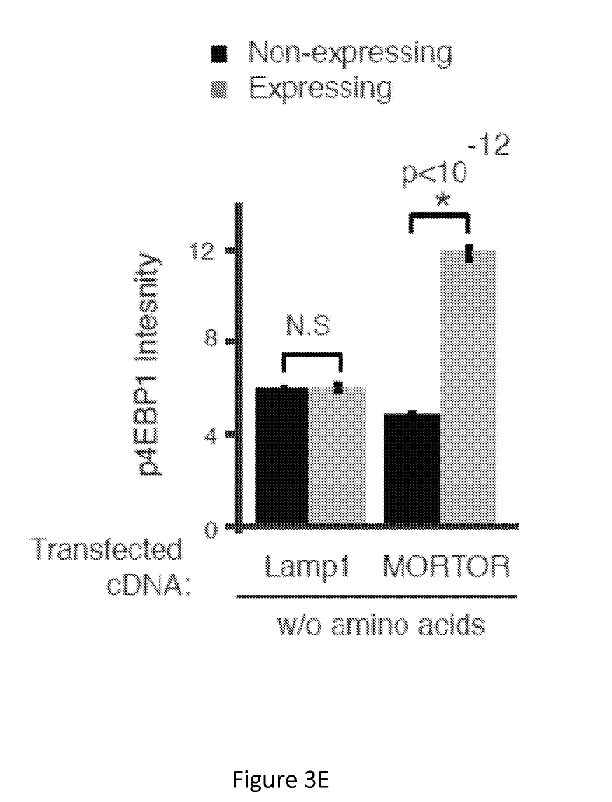

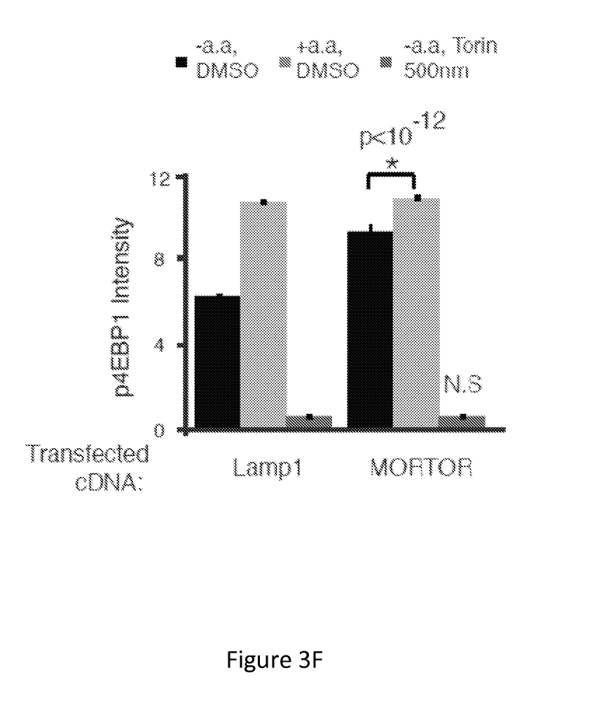

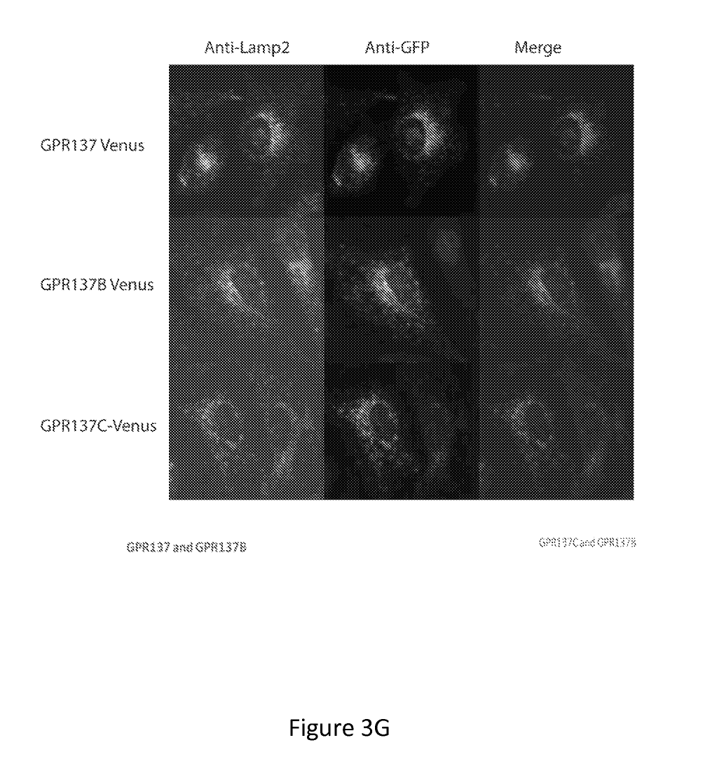

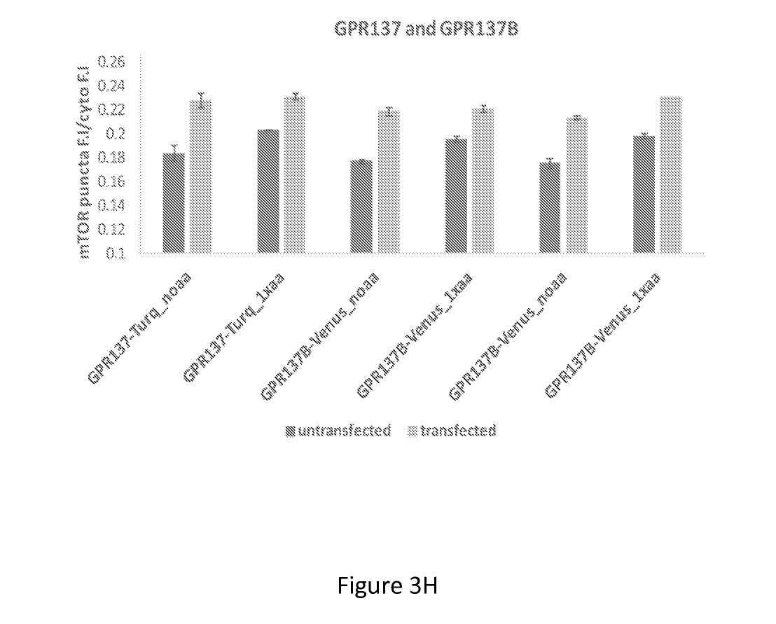

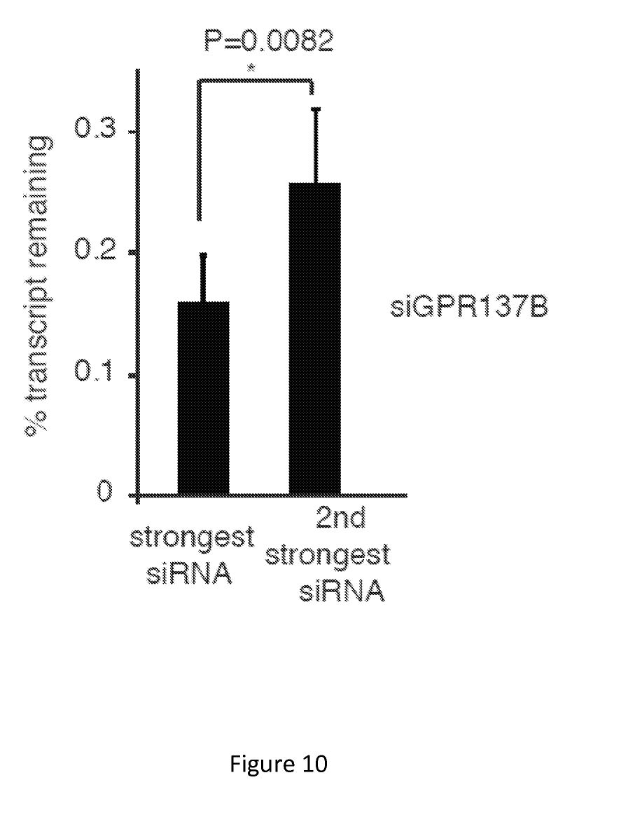

A number of control experiments were performed to ensure that a regulator of amino acid signaling to mTORC1 had been identified. Knockdown of MORTOR using 2 different synthetic siRNAs (siGPR137B #1 (SEQ ID NO:3) and siGPR137B #2 (SEQ ID NO:4)) reduced mTOR translocation to lysosomes and rpS6 phosphorylation (FIG. 3A; in response to cycloheximide addition to serum starved cells). The knockdown of MORTOR at the transcript level was confirmed by qPCR using the same synthetic siRNAs (FIG. 10). In the corollary experiment, overexpression of MORTOR caused increased mTOR translocation to lysosomes in response to amino acid stimulation (FIG. 3C). To rule out the siRNA off-target effect, a rescue experiment was performed demonstrating that the translocation of mTOR to lysosomes can be suppressed by an siRNA targeting the 3′UTR of MORTOR and that the mTOR translocation can then be restored by overexpression of MORTOR (FIG. 3D).

Given that mTORC1 has a role in suppressing autophagy, we next determined whether MORTOR knockdown increases autophagy. Mammalian TORC1 inhibits autophagy by preventing induction of autophagosome formation through phosphorylation of the ULk1-mATG13-FIP200 complex (Chan, E. Y. (2009) “MTORC1 Phosphorylates the ULK1-mAtg13-FIP200 Autophagy Regulatory Complex,” Science Signaling, 2(84):pe51 (doi: 10.1126)). Indeed, MORTOR knockdown caused a striking upregulation of autophagy in full serum condition as measured by the number of LC3-GFP puncta per cell (FIG. 3D). We further tested whether MORTOR can regulate the mTORC1-mediated phosphorylation and inhibition of the translation repressor 4EBP1. 4EBP1 phosphorylation requires less mTORC1 activity since amino acid deprivation only partially suppresses 4EBP1 phosphorylation (FIG. 3E). As a control, the mTOR inhibitor Torin completely suppressed 4EBP1 phosphorylation (FIG. 3F). Interestingly, overexpression of MORTOR in amino acid starved cells caused a near maximal increase in the phosphorylation of 4EBP1 close to the level reached by amino acid stimulation (FIG. 3F). This suggests that only a relatively low level of mTORC1 activity is required to keep 4EBP1 phosphorylated and that overexpression of MORTOR can increase mTORC1 signaling towards 4EBP1 to a near maximal level even in the absence of amino acids (FIG. 3F). Together, this shows that MORTOR is a regulator of amino acid-induced mTORC1 translocation to lysosomes and thereby controls mTORC1-mediated activation of S6 kinase and 4EBP1 as well as suppression of autophagy.

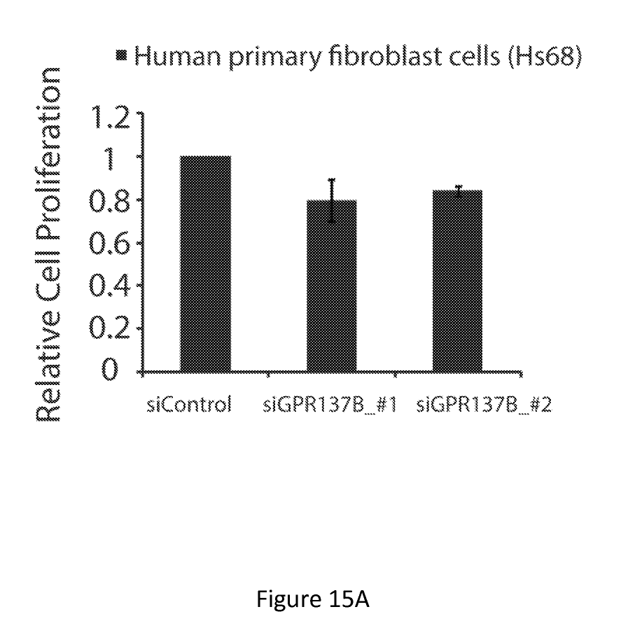

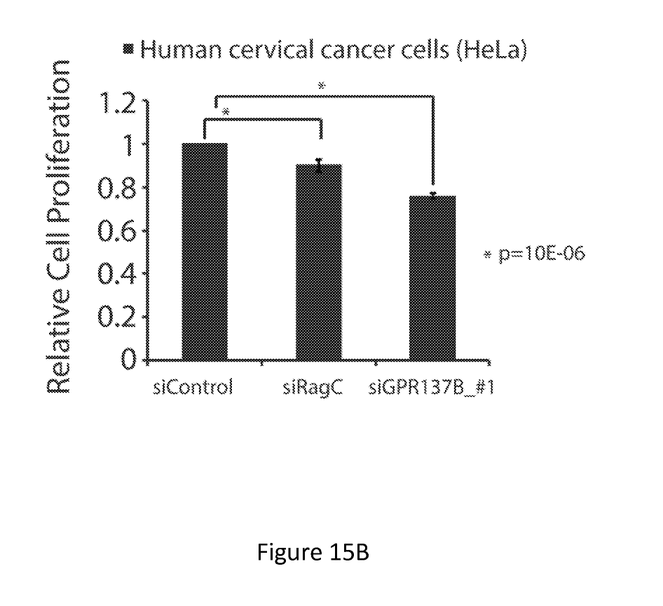

Knockdown of MORTOR also reduced cell proliferation of a primary human fibroblast cell line, Hs68, and a human cervical cancer cell line, HeLa. Hs68 cells were transfected with siRNAs using Lipofectamine 2000 according to the manufacturer's instructions. Similarly, HeLa cells were transfected with siRNAs using the GENESILENCER siRNA transfection reagent according to the manufacturer's instructions. 40 to 48 hours after transfection, cells were fixed and counted. Proliferation of cells transfected with an siRNA against MORTOR (siGPR137B #1 or siGPR137B #2) was compared with that of cells transfected with a control siRNA (negative control) or an siRNA against RagC, a direct regulator of mTORC1 translocation (positive control). The siRNAs against MORTOR significantly inhibited cell proliferation of both the human primary fibroblasts (FIG. 15A) and the human cervical cancer cells (FIG. 15B) compared to controls.

MORTOR is Localized to Lysosomal Membranes

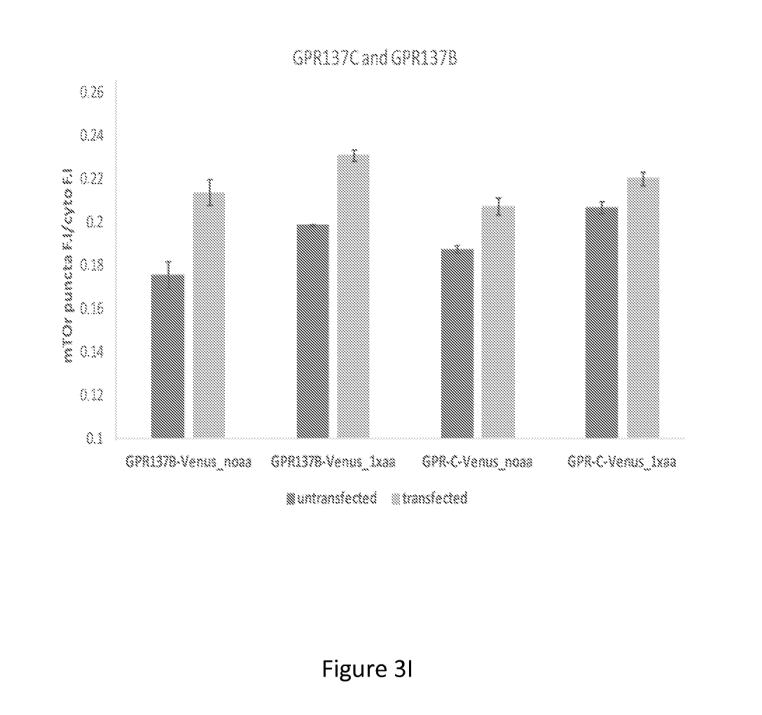

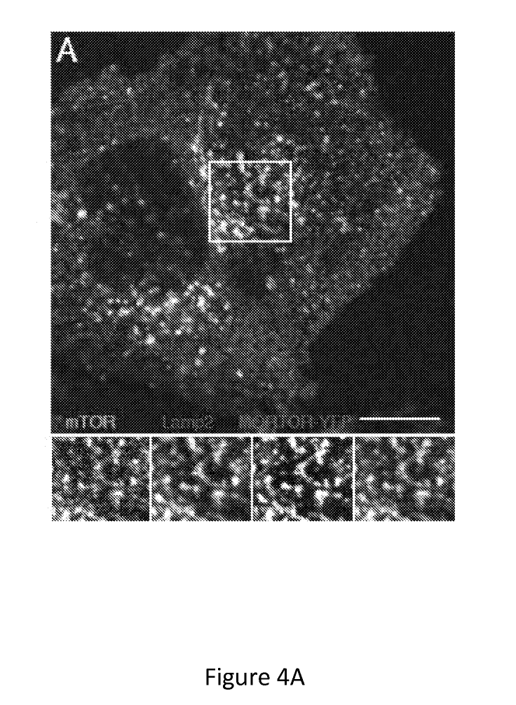

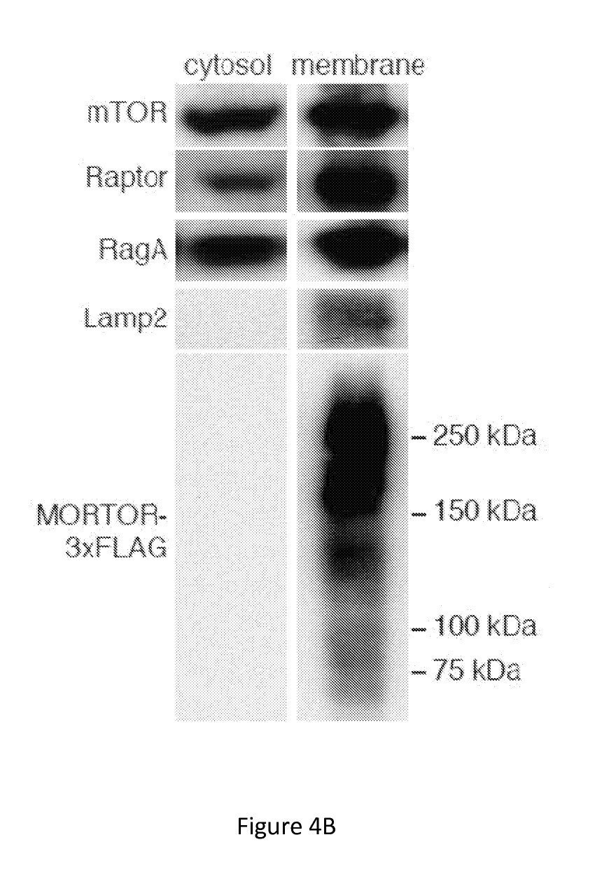

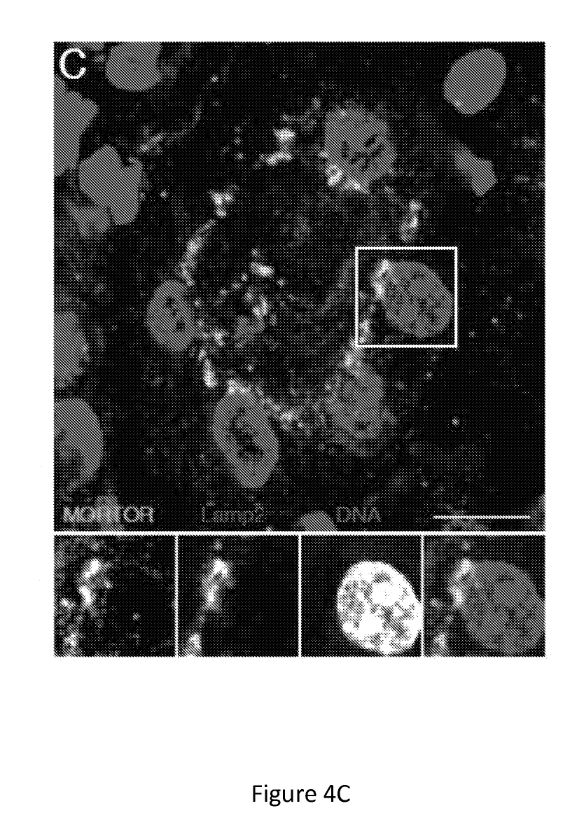

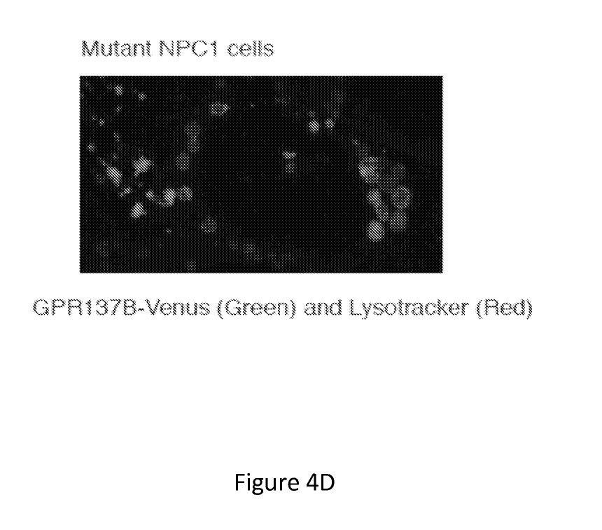

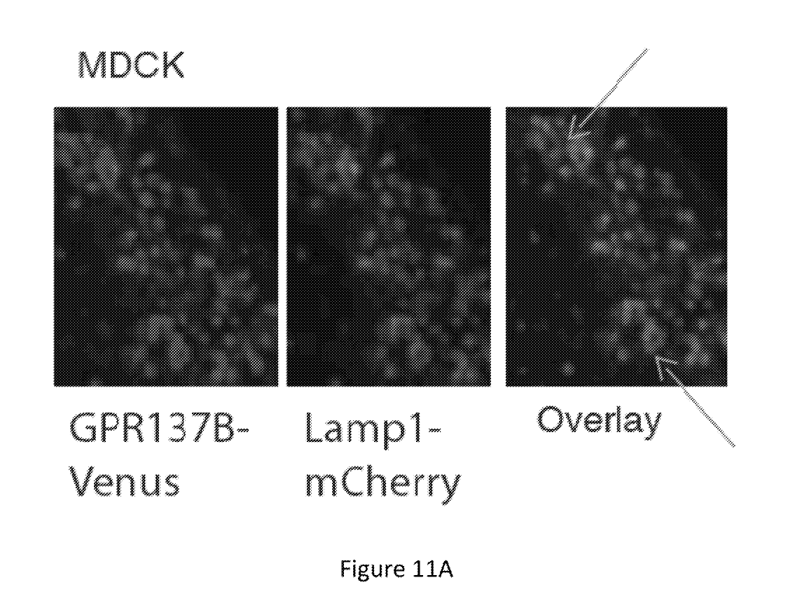

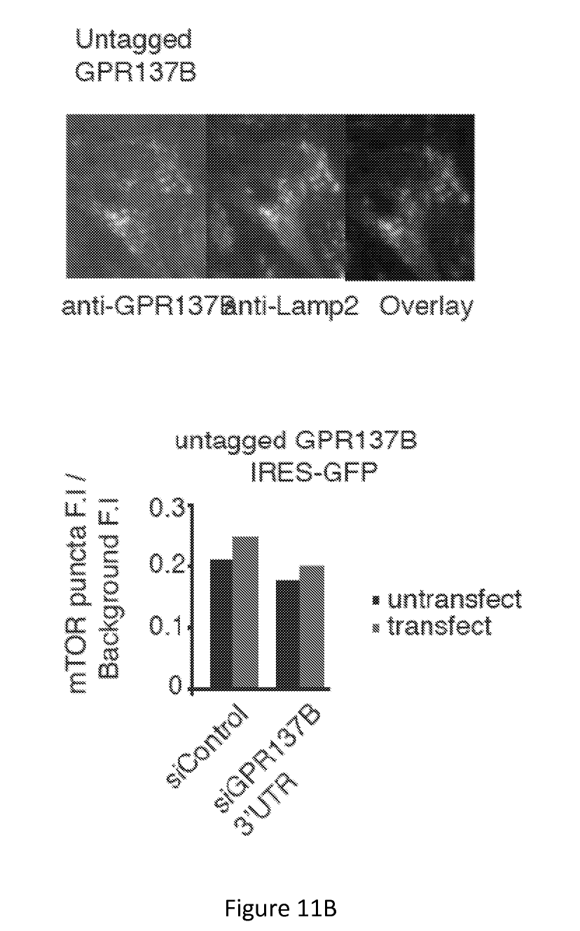

We next visualized the expression of YFP-conjugated MORTOR protein to confirm the previous observation that MORTOR is a lysosomal membrane protein (Gao, J., et al. (2012) “TM7SF1 (GPR137B): a Novel Lysosome Integral Membrane Protein,” Journal of Molecular Biology, 39:8883-8889). Consistent with the previous finding, expressed MORTOR-YFP localized to vesicles marked by the lysosomal protein Lamp2. Furthermore, in the presence of amino acids, MORTOR also co-localized with vesicles stained by antibodies against endogenous mTOR (FIG. 4A). As further validation, YFP-conjugated MORTOR also localized to lysosomes in MDCK cells (FIG. 11A), and HeLa cells expressing untagged MORTOR and stained with an endogenous antibody against MORTOR co-localized with endogenous Lamp2 as well (FIG. 11B).

Fractionation of cells expressing MORTOR-3×FLAG showed that MORTOR was exclusively in the membrane containing fraction, unlike endogenous RagA, mTOR or Raptor which were mostly in the cytosolic fraction (FIG. 4B). Next, we made use of NPC-1 deficient CHO cells which have defective lysosome trafficking that causes enlarged late endosomes and lysosomes, allowing for the membrane of lysosomes to be visualized by light microscopy (FIG. 4D). Expression of MORTOR-YFP confirmed that MORTOR was predominantly localized in the membrane of lysotracker-positive lysosomes. Finally, we tested for the localization of endogenous MORTOR. Since mRNA expression of MORTOR has been shown to be high during kidney development (Spangenberg et al, (1998) “Cloning and Characterization of a Novel Gene (TM7SF1) Encoding a Putative Seven-Pass Transmembrane Protein that is Upregulated During Kidney Development,” Genomics 48: 178-185), we used mouse kidney sections and found that an antibody targeting endogenous MORTOR protein co-localized with Lamp2, suggesting that MORTOR also localizes to lysosomes in vivo (FIG. 4C). While this antibody was sufficiently sensitive to recognize endogenous kidney MORTOR and overexpressed lysosomal localized MORTOR, it was not able to detect vesicular localization of endogenous proteins in HS68, Hela, and HEK293 cells that we tested. Of note, overexpressed MORTOR-YFP localized nearly exclusively to lysosomes while overexpressed RagGTPases or Lamtor1 was less selectively localized to lysosomes and was also found in other cell compartments. This raised the possibility that the lysosome specific localization of MORTOR may in part contribute to lysosome selective mTORC1 signaling.

MORTOR Acts Before Rag Heterodimers in Regulating mTORC1 Translocation

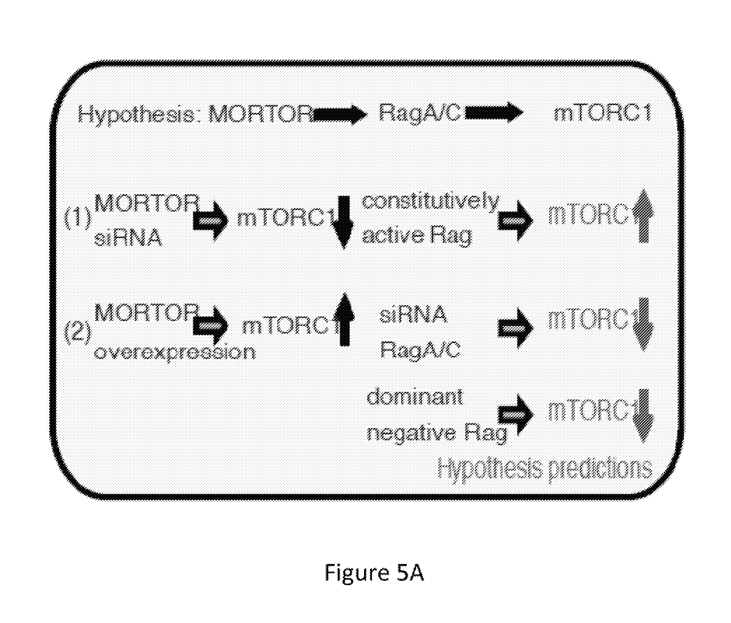

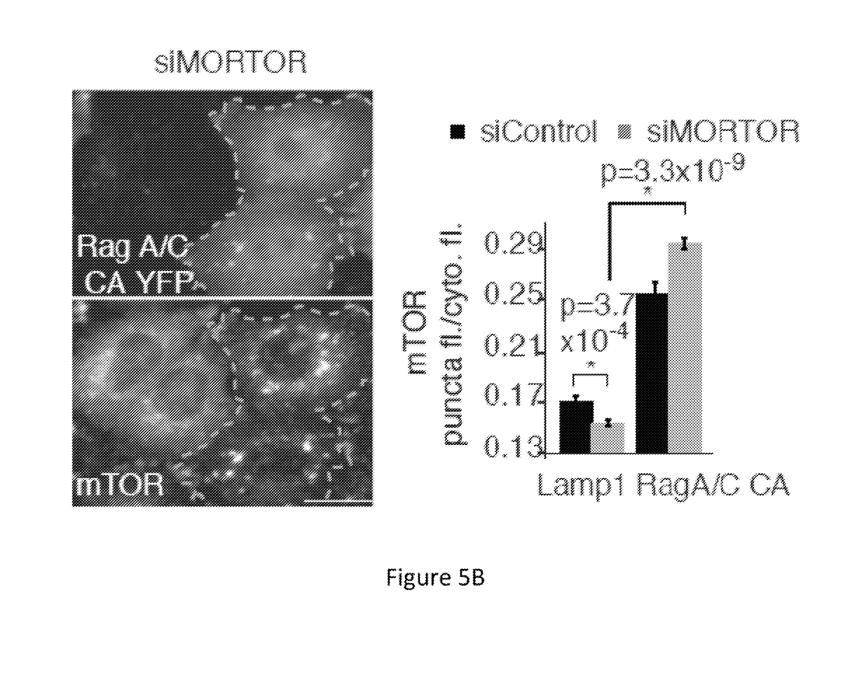

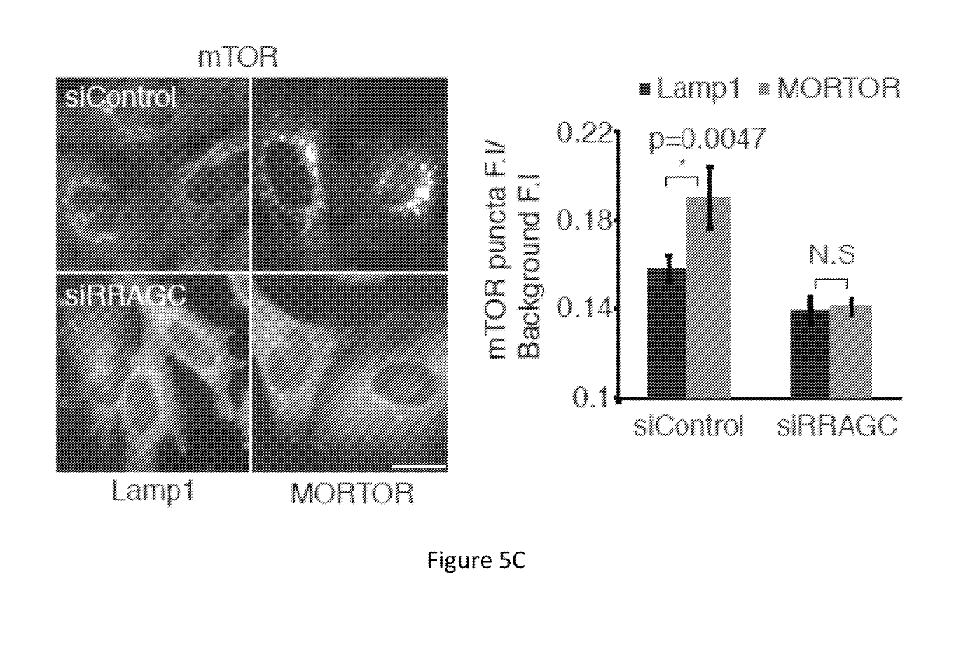

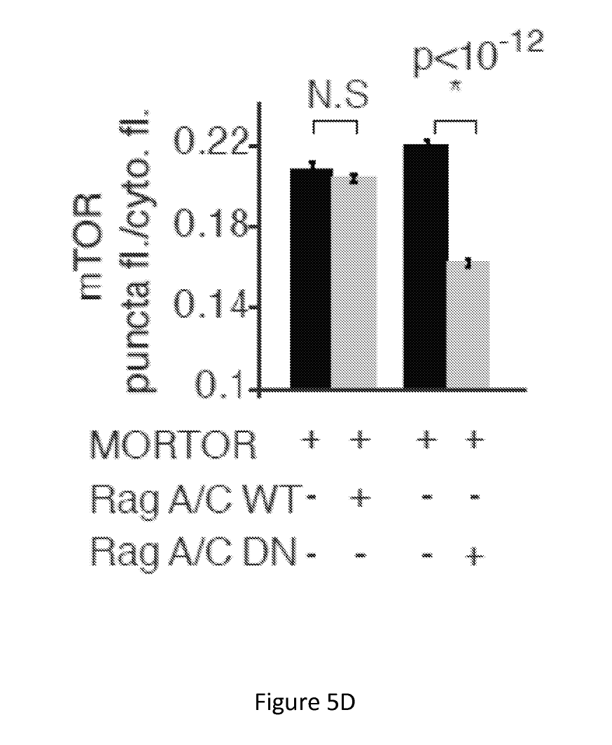

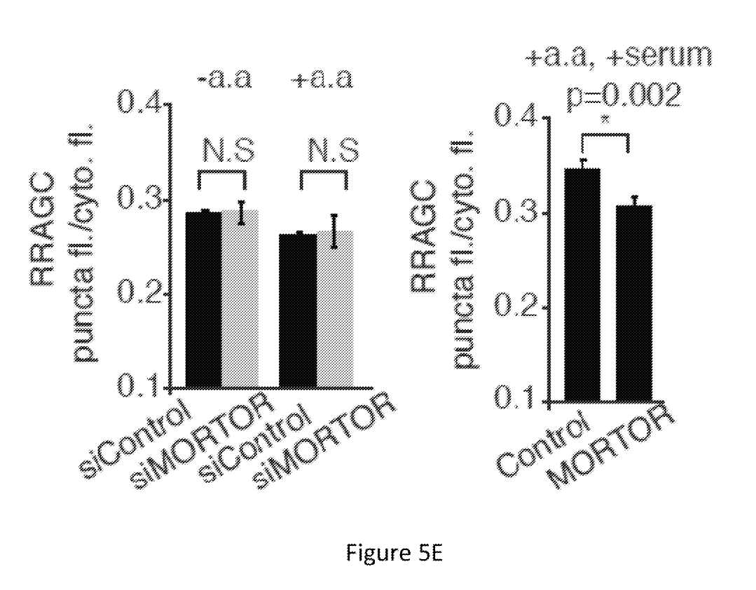

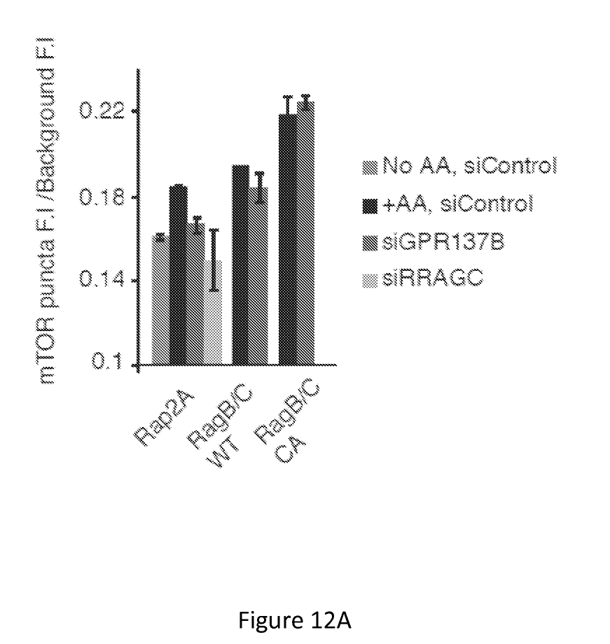

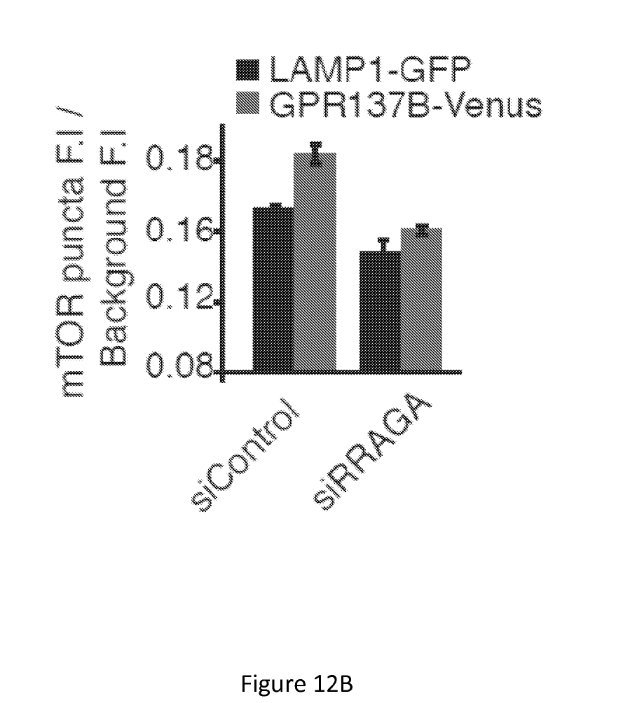

Heterodimeric lysosomal Rag GTPases bind directly to the mTORC1 complex protein Raptor if RagA or its homolog RagB is in the GTP loaded state (Sancak, Y., et al. (2008) “The Rag GTPases Bind Raptor and Mediate Amino Acid Signaling to MTORC1,” Science, 320:1496-1501). Consistent with a key role of Rag heterodimers, our screen found RRAGC siRNAs to be the strongest suppressors of amino acid-induced mTOR translocation to lysosomes (FIG. 2C). Subsequent siRNA analysis showed that knockdown of RagA also reduces mTORC1 translocation (FIG. 12B). We used siRNA knockdown and overexpression of dominant negative and constitutively active versions of Rag heterodimers to test whether MORTOR acts upstream from the RagGTPases. FIG. 5A depicts the predicted results from the experiments we performed to test whether MORTOR may act upstream of Rag. Knockdown of MORTOR was not able to suppress the increase in lysosomal localization of mTOR in cells expressing constitutively active versions of RagA/C (FIG. 5B). The same result was observed when we expressed a constitutively active RagB/C pair (FIG. 12A). Moreover, overexpression of high levels of MORTOR strongly enhanced mTOR translocation to lysosomes even in amino acid deprived cells. This relative increase could be completely suppressed by knocking down RagC (FIGS. 5C and 12B). Interestingly, expression of MORTOR together with wild-type RagA/C did not further increase or decrease mTOR translocation to lysosomes. However, expression of MORTOR together with a dominant negative RagA/C pair caused a significant reduction in mTOR recruitment (FIG. 5D). Together, these results argue that MORTOR is positioned upstream of Rag heterodimers in the lysosomal amino acid signaling pathway to mTORC1.

As a potential indirect regulatory mechanism, we considered whether MORTOR may regulate the concentration of Rag heterodimers at lysosomes. To examine such a potential indirect regulatory mechanism, we used immunofluorescence to quantify the amount of endogenous RagC localized to lysosomes. No significant difference in the amount of endogenous lysosomal RagC was observed in MORTOR knockdown cells compared to negative control cells either in the presence or absence of amino acids (FIG. 5E, left). We also did not find more endogenous RagC localized to lysosomes in cells overexpressing MORTOR. There was instead a small reduction in Rag localization (FIG. 5E). Together, this argued against an indirect role of MORTOR in increasing the lysosomal localization of Rag hetero dimers.

Amino Acid-Stimulates Binding of MORTOR to the Rag/mTORC1 Complex

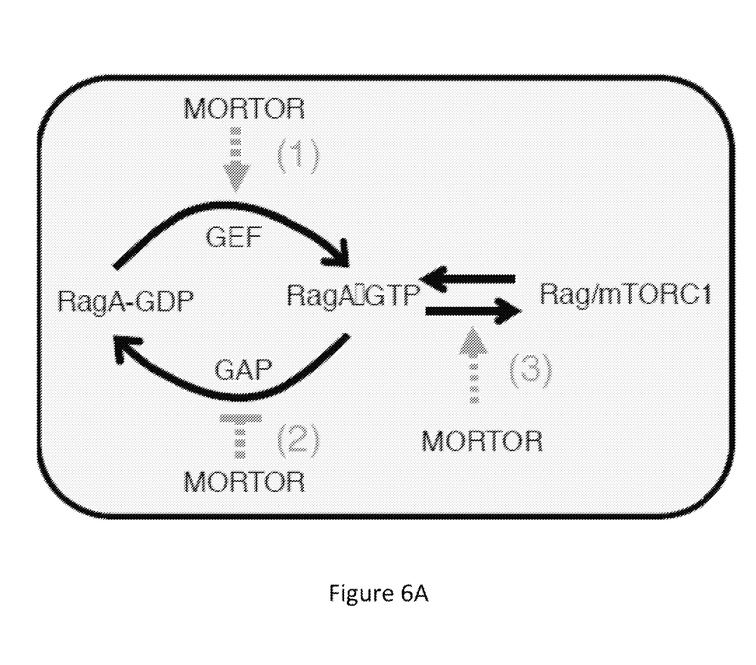

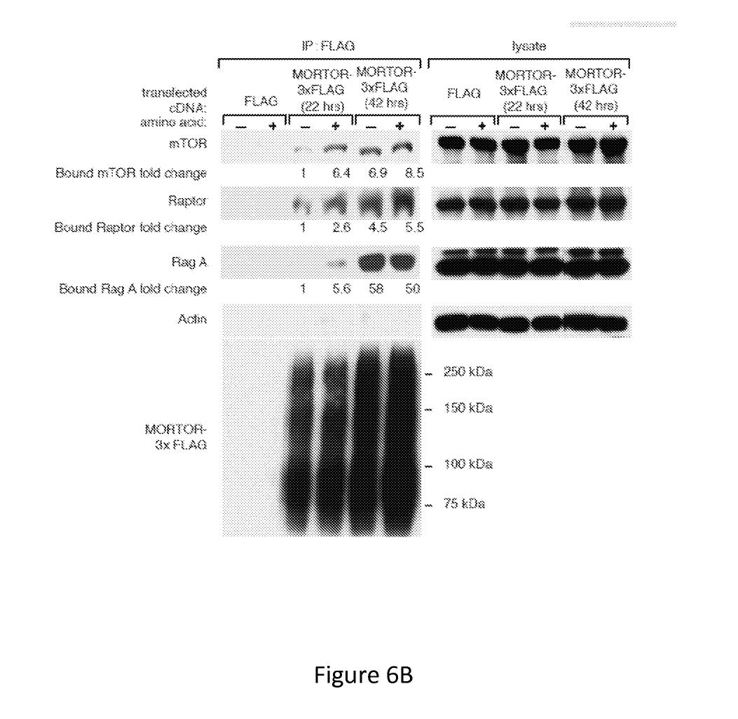

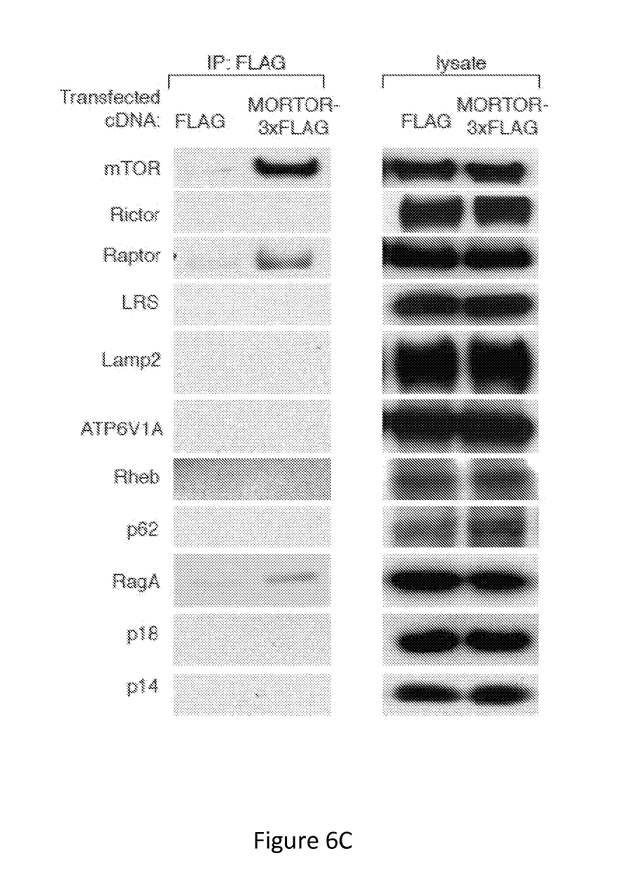

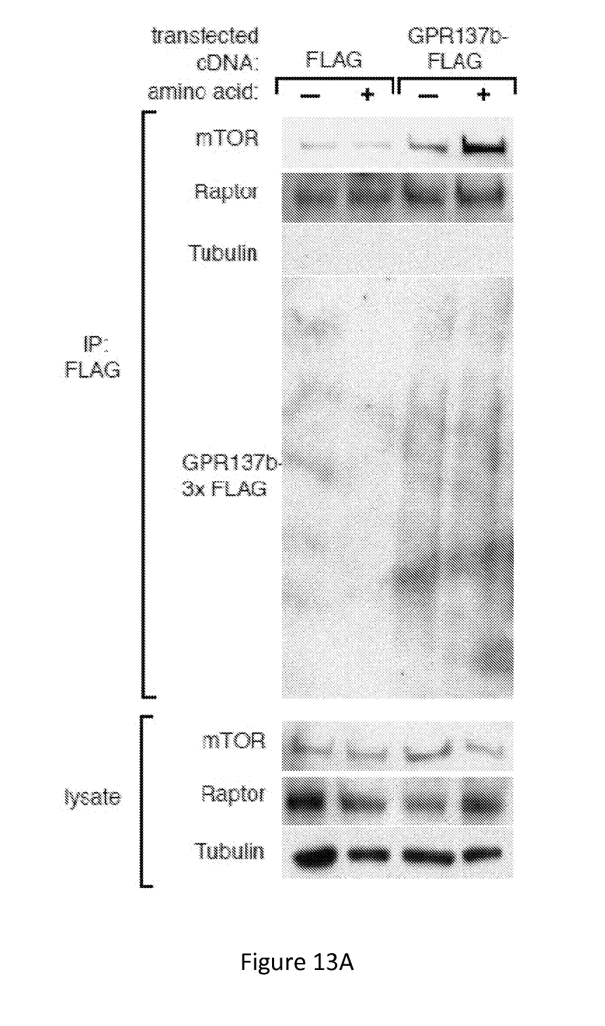

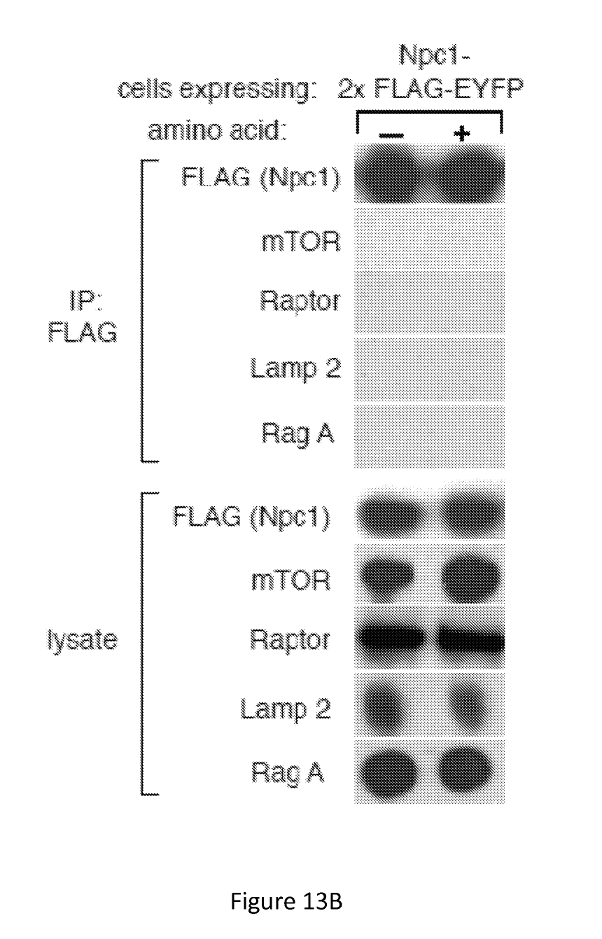

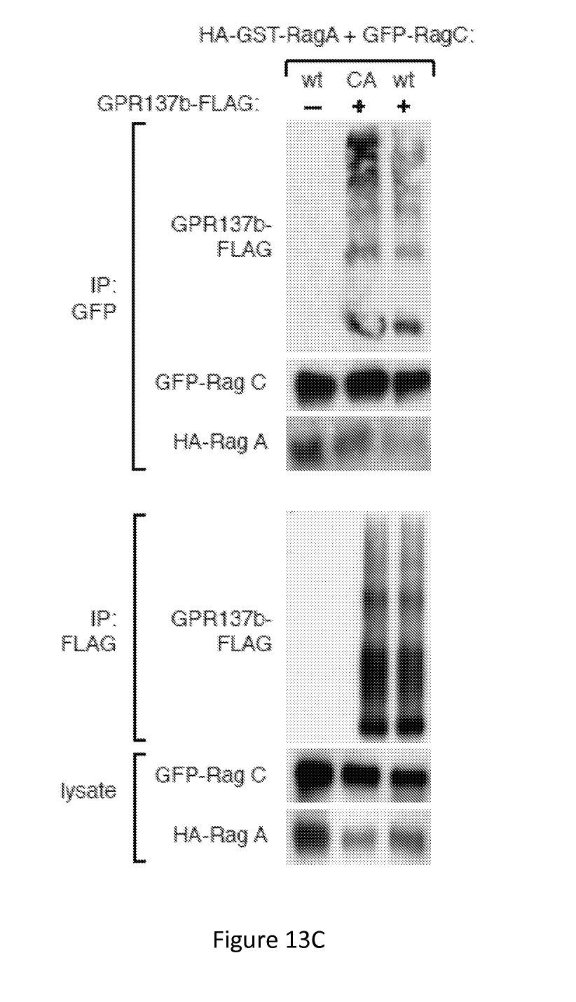

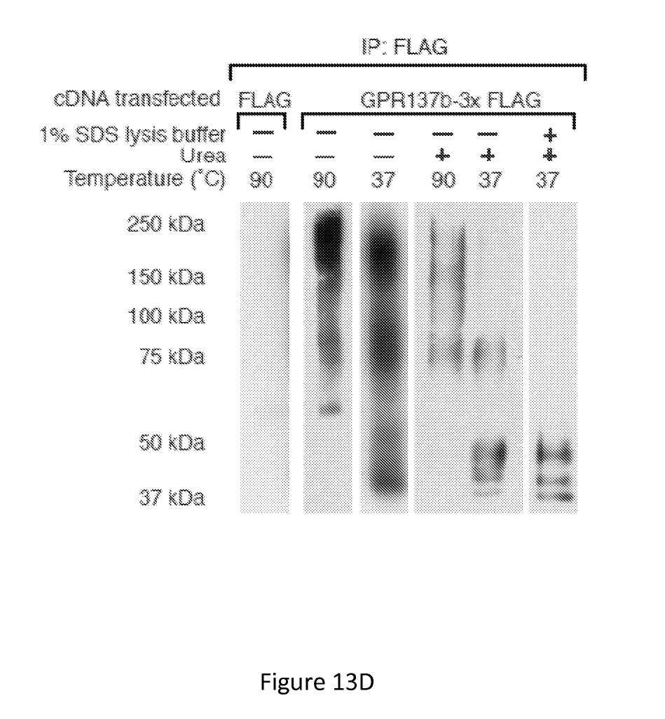

Since MORTOR is localized to lysosomes and acts before Rag, we considered that MORTOR may regulate mTORC1 translocation by either increasing the Rag A GDP/GTP exchange activity, by suppressing RagA GTPase activity or by stabilizing a RagA-GTP/mTORC1 complex (FIG. 6A). To learn whether MORTOR plays one of these roles, we first investigated whether MORTOR interacts with Rag GTPases or mTORC1. Markedly, endogenous Rag A, mTOR, and Raptor all co-immunoprecipitated along with MORTOR-FLAG in lysates of HEK 293T cells (FIG. 6B). Not only did MORTOR pull down these mTORC1 and Rag components, the amount of mTOR, Raptor and RagA was also several-fold higher following amino acid stimulation. The same amino-acid dependent interaction of MORTOR with mTOR, RagA and Raptor was also observed in HeLa cells (FIG. 13A). The residual interaction in the absence of amino acids matched a partial mTOR translocation to lysosomes in cells expressing MORTOR (FIG. 3B).

The MORTOR interactions was likely specific for mTORC1 and Rag components as we did not detect Rictor, the Ragulator components Lamtor1-3, LRS, ATP6V1A, Rheb or the abundant lysosomal Lamp2 protein in the co-immunoprecipitates. This suggests that MORTOR has a regulatory role that not only involves an interaction with RagA but also with mTORC1 (FIG. 6C). In an additional control experiments, co-immunoprecipitation in HEK293 Ts cells using the same pull down conditions but stably-integrated FLAG tagged NPC1, a 13 transmembrane lysosomal protein, did not yield any mTORC1 components or RagGTPases bound (FIG. 13B). This suggests that binding to mTORC1 is not a universal feature of lysosomal membrane spanning proteins. Furthermore, reverse pulldowns using GFP-tagged RagA/Cs further confirmed that RagA/C interacts with MORTOR in HEK293T cells (FIG. 13C). On a technical note, over-expressed MORTOR requires relatively high levels of detergent to be extracted from membranes and it migrates as multiple bands in an SDS-PAGE possibly due to residual aggregation of higher molecular weight complexes which is common in multi-transmembrane proteins. (Zou C, et al. (2008) “Biosynthesis and NMR-studies of a double transmembrane domain from the Y4 receptor, a human GPCR.” J Biomol NMR, 42(4):257-69; see FIG. 13D).

Together, these studies indicate that MORTOR has not only a signaling role of increasing the Rag interaction with mTORC1, but also a scaffolding function of bridging RagA, Raptor and mTOR, thereby retaining mTORC1 complexes at lysosomes. This excludes our original hypothesis that MORTOR is not exclusively an activator of a Rag A exchange factor or a suppressor of a Rag A GTPase (FIG. 6A); both these mechanisms would not require for MORTOR to interact with mTORC1. Instead, this suggests that MORTOR also has a role in assembling a MORTOR/Rag/mTORC1 signaling complex in response to amino acid stimulation.

Dual Roles of MORTOR as an Upstream Rag Regulator and as an Adaptor in a MORTOR/Rag/mTORC1 Signaling Complex

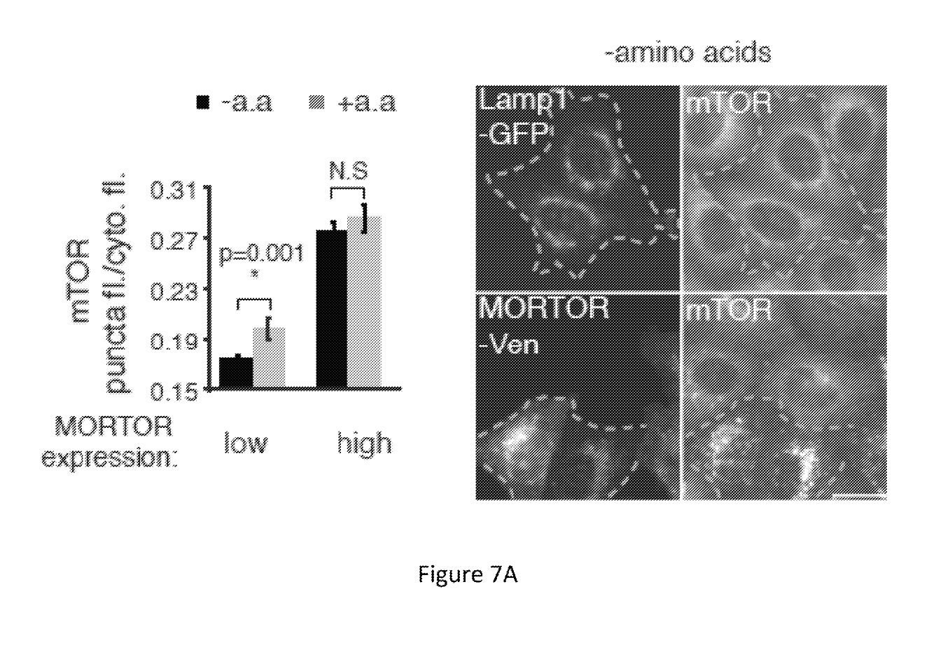

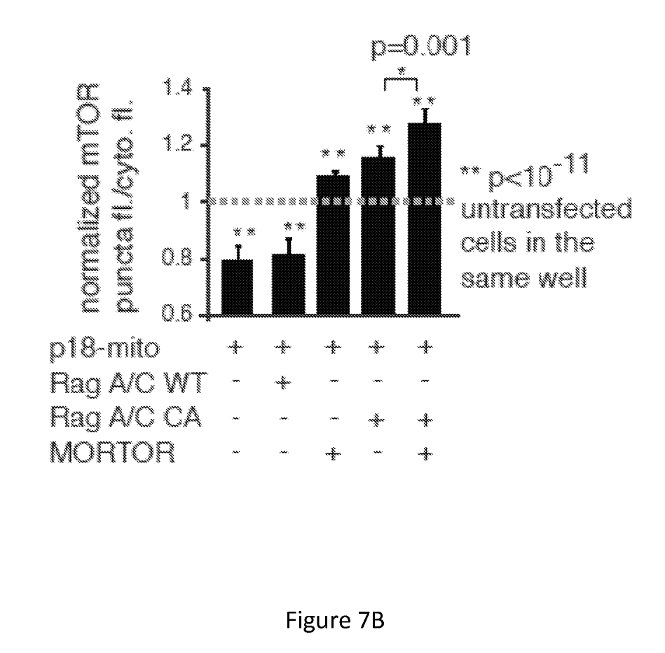

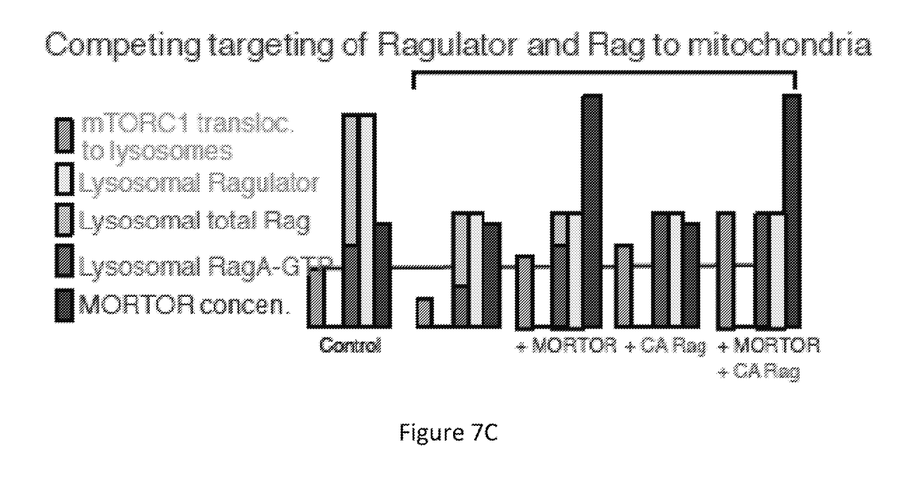

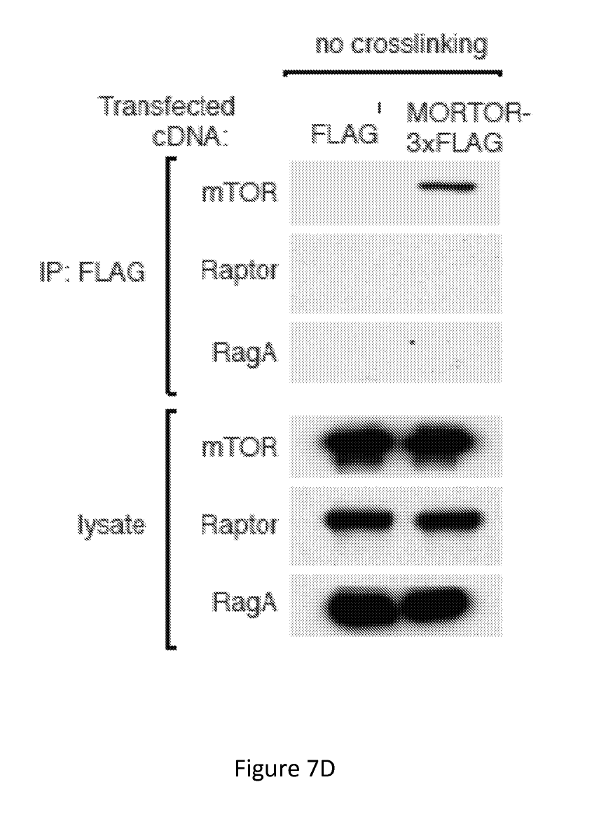

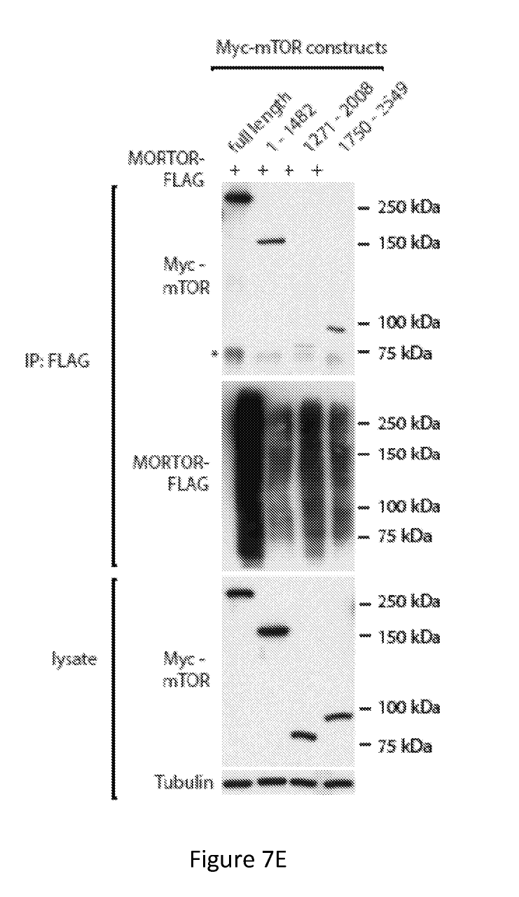

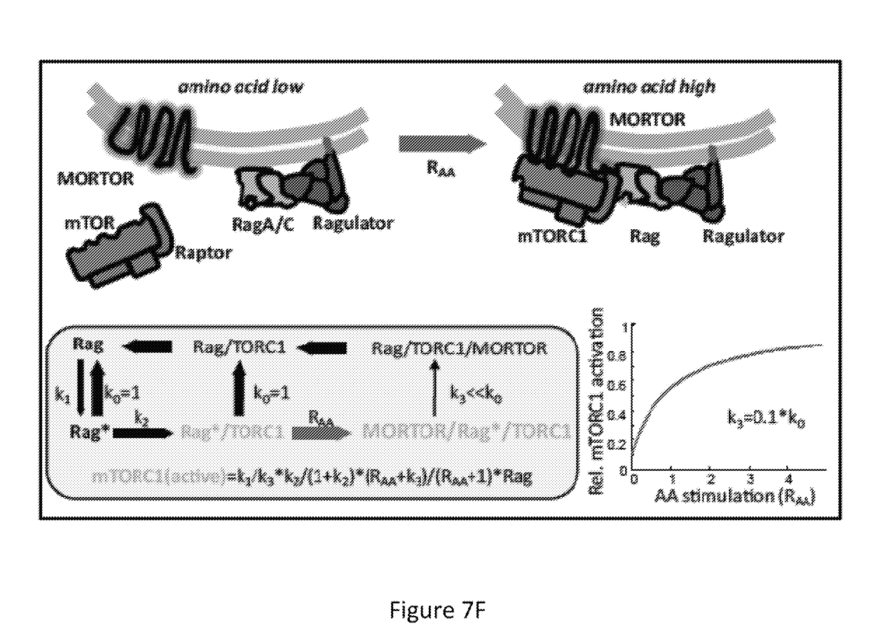

Expression of low or moderate levels of MORTOR induced a small increase in mTOR translocation to lysosomes already in the absence of amino acids that could then be amplified by amino acid stimulation. At higher levels of MORTOR expression, mTOR translocation to lysosomes and 4EPB1 phosphorylation reached levels similar to those of amino acid stimulated cells even in the absence of amino acids (FIG. 7A; FIGS. 3E and 3F). In HEK293T cells overexpressing MORTOR for 42 hours (to reach higher expression levels), cells starved of amino acids had also similar amounts of mTOR co-immunoprecipitating with MORTOR, whether cells were stimulated with amino acids or not (FIG. 6A). This suggests that high expression of MORTOR renders cells largely insensitive to amino acids in terms of mTORC1 translocation, mTORC1 activity towards 4EBP1 as well as MORTOR binding to Rag/mTORC1.

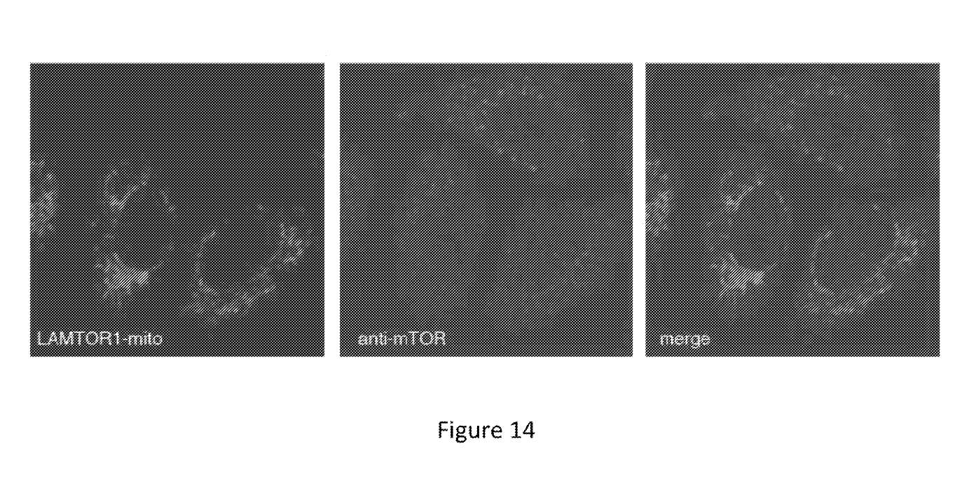

Since endogenous Rag GTPases are required for MORTOR to mediate mTORC1 recruitment (FIG. 5), this suggested that MORTOR overexpression caused partial RagA GTP loading even in the absence of amino acids. We therefore performed a series of experiments to assess the relative roles of MORTOR, Rag GTPases, and Ragulator in mediating mTORC1 recruitment. Our approach was to reduce the concentration of ragulator and Rags at lysosomes by mis-targeting the Ragulator component Lamtor1 by fusing it to a mitochondrial targeting sequence and expressing the construct in HeLa-cells (Lamtor1-mito) (Sancak, Y., et al. (2010) “Ragulator-Rag Complex Targets MTORC1 to Lysosomal Surface and is Necessary for its Activation by Amino Acids,” Cell, 141:290-303). Consistent with the result by Sancak and coworkers, we did not see mTOR localize to mitochondria in Lamtor1-mito expressing cells (FIG. 14A), but we did observe a significant decrease in the amount of mTOR localized to lysosomes when compared to untransfected cells in the same well (FIG. 7B). This is likely due to mitochondrial Lamtor1 sequestering endogenous Rag GTPases and Ragulator components and depleting them from lysosomes, thereby decreasing the efficiency of amino acid stimulated mTORc1 recruitment to lysosomes (Sancak, Y., et al. (2010) “Ragulator-Rag Complex Targets MTORC1 to Lysosomal Surface and is Necessary for its Activation by Amino Acids,” Cell, 141:290-303). It was interesting that this reduced lysosomal translation of mTOR was limited by the reduced level of lysosomal Ragulator, as over-expression of wildtype RagA/C did not increase mTORC1 recruitment (FIG. 7B).

We used this system to perform three competition experiments to test the hypothesis that mTORC1 translocation to lysosomes is mediated by MORTOR-mediated GTP loading of RagA and retention of RagA-GTP/mTORC1 complexes at lysosomes. Particularly, we tested whether the over-expression of either (1) MORTOR, (2) constitutively active RagA/C, or (3) MORTOR plus CA Rag A/C can compensate for the loss in mTORC1 translocation caused by the co-expression of mitochondrial localized Lamtor1. Consistent with a model that high levels of MORTOR can convert Rag-GDP to Rag-GTP, MORTOR expression more than restored mTORC1 recruitment to lysosomes even though the total number of Ragulator/Rag complexes was suppressed at lysosomes. Furthermore, overexpression of RagA/C CA was able to cause an equally strong increase in lysosomal mTOR translocation even though the Ragulator was depleted, suggesting that only a relatively small fraction of the RagA has to be normally converted into the GTP state for maximal mTORC1 translocation. The mTOR localization to lysosomes could then be further increased by also expressing high levels of MORTOR (FIG. 7B). Under these limiting conditions, MORTOR acts synergistically with constitutively active Rag, suggesting that the MORTOR/Rag/mTORC1 complex binds with higher affinity to lysosomes than a Rag/mTORC complex alone. FIG. 7C shows a schematic interpretation of how the lysosomal RagA-GTP level may change during these three perturbation protocols. These compensation experiments are consistent with a dual role of MORTOR in increasing the RagA-GTP concentration after amino acid stimulation and at the same time acting as an adaptor protein that maintains an active MORTOR/Rag/mTORC1 complex at the lysosomes as long as amino acid signals remain elevated.

As a further indication that the formation of a MORTOR/Rag/mTORC1 complex is based on a high affinity interaction, expressed MORTOR binding to endogenous mTOR can readily be detected even in the absence of cross-linkers (FIG. 7D). Our previous interaction experiments between raptor and mTOR or RagA and raptor, as well as the interactions reported by other groups), are all performed in the present of crosslinking reagents (FIG. 6B,C and Kim, D. H. et al (2002) “mTOR Interacts with Raptor to Form a Nutrient-Sensitive Complex that Signals to the Cell Growth Machinery,” Cell 110: 163-175). The relatively high affinity interaction between MORTOR and mTOR likely involves more than one binding site as we found that MORTOR interacted with both an N and a C-terminal fragment of mTOR (FIG. 7E). Without crosslinking reagent, Raptor and Rag A could not be detected in the co-immunoprecipitates. This finding leads to a model that MORTOR functions in assembling a stable complex that sustains mTORC1 signaling as long as amino acids stay elevated.

Discussion

Genome-Wide Screen Identifies a Modular Architecture of the Amino Acid Controlled mTORC1 Signaling System

We conducted a genome wide screen to identify groups of regulators that control the amino acid sensing signaling pathway. Our assay was set up to identify, in addition to amino acid signaling regulators, also regulators of the PI3K/Akt/TSC2/Rheb signaling pathway, which acts in parallel and provides a key co-stimulatory input for TORC1-mediated S6 kinase activation (FIG. 1A). The statistical pathway analysis of our primary screen included, in an addition to the mTOR module itself, also the insulin, GPCR, Erk/MAPK, and PI3K/Akt signaling modules known to be linked to cell growth. Interestingly, our validated hits were also enriched for genes in the stress modules p53 and AMPK and in the cell cycle and hedgehog signaling modules. This argues that stress and proliferation are closely linked to the mTOR-mediated regulation of cell growth. The identified genes in the urea cycle and telomerase modules are likely linked to the stress pathway, since effective urea metabolism and telomerases are critical for cell health in growing cells. We also uncovered a number of additional regulatory modules that were less expected and that linked to amino acid metabolism, particularly genes in the asparagine biosynthesis and the glycine cleavage pathway which strongly regulated rpS6 phosphorylation and mTORC1 translocation. These modules likely reflect rate-limiting synthesis of particular amino acids that in turn change mTORC1 activity and may provide a means to use selective amino acid synthesis enzyme inhibitors to specifically regulate mTORC1 function. We also found that the knockdown of many ribosomal proteins increases rpS6, which can be explained by a rapid feedback that links reduced ribosomal activity to increased rpS6 phosphorylation (as can be shown by addition of the translation inhibitor cyclohexamide). This rapid mTORC activity increase occurs since the synthesis of amino acids continuous while the removal through protein synthesis has stopped or slowed down, triggering an increase in free amino acids. In summary, our statistical pathway analysis suggested interesting new links to mTORC1 from known cellular regulatory modules. Our screen also identified many potentially interesting genes that do not yet have known functions but that can likely be assigned to some of these regulatory pathways and characterized in molecular detail using additional more focused cellular assays.

Mechanistic Model of Amino Acid-Induced Activation of MORTOR, Rag and mTORC1

We focused the follow-up work from the screen on our discovery of the seven transmembrane protein MORTOR that had prior to our study no known function. We demonstrated that MORTOR is a necessary signaling component in the amino acid sensing pathway leading to mTORC1 translocation and activation. Specifically, we showed that MORTOR knockdown suppresses mTOR translocation to lysosomes and overexpression causes increased mTOR recruitment to lysosomes in response to amino acids and, at high expression levels, even in the absence of amino acids. At these high expression levels, the mTOR substrate 4EBP1 was also already maximally phosphorylated in the absence of amino acids. We further showed that MORTOR knockdown can be fully rescued by constitutively active RagA/C GTPases, and that MORTOR mediated mTOR recruitment is abolished by Rac C knockdown, reduced by RagA knockdown and suppressed by expression of dominant negative RagA. Consistent with a previous report, we also confirmed that MORTOR is exclusively localized in the lysosome membrane. Together, these findings argued that MORTOR functions as an upstream mediator of amino acid signaling to Rag and mTORC1.

Our biochemical analysis showed that MORTOR interacts with mTOR, raptor and RagA and that this interaction is strongly increased by amino acid stimulation. The interaction of MORTOR with mTOR appeared to be particularly tight as the mTOR-MORTOR interaction could be detected without cross-linking and involved both C- and N-terminal regions of mTOR. We also performed a competition analysis where we expressed a mitochondrial localized Lamtor1 that pulled some of the Ragulator and Rag but not mTORC1 from lysosomes to mitochondria, allowing us to better understand the role of MORTOR in Rag and mTORC1 translocation. The results from these experiments were best explained by increased MORTOR expression increasing the Rag-GTP concentration at lysosomes. Furthermore, MORTOR was able to further increase mTORC1 translocation even when an excess of constitutively active RagA-GTP was expressed together with RagC-GDP, suggesting that MORTOR assembles a high affinity complex between MORTOR, Rag and mTORC1. Collectively, these results show MORTOR is an essential mediator of amino acid induced mTORC1 translocation and activation that functions upstream of Rag by increasing the RagA-GTP load as well as downstream of Rag by assembling a persistent lysosomal MORTOR/Rag/mTORC1 signaling complex that remains active as long as amino acid levels remain elevated.

How can the same protein regulate Rag-GTP loading and also bind and stabilize the RagA-GTP-mTORC1 complex? It is possible that MORTOR has first a role as a GEF enhancer or a GAP suppressor and then a second role in assembling the Rag-GTP/mTORC1 complex. Alternatively, MORTOR functions as a trap by slowing the Rag-GTP hydrolysis when binding the Rag-GTP/mTORC1 complex. In principle such a trapping mechanism can function by protecting the RagA-GTP state without any need for a regulated GEF or GAP activity as long as the basal GEF activity is smaller than the basal GAP activity. FIG. 7F shows a simplified model of how such a trapping mechanism may work. The key parameter in the overall equation is the degree of slow-down in the RagA-GTP hydrolysis rate upon binding of MORTOR (k₃/k₀). The equation and corresponding simulation shows an example how this trapping mechanism can increase mTORC1 recruitment to lysosomes in response to amino acid stimulation. In the shown case, the MORTOR binding to RagA-GTP/mTORC1 was assumed to slow the GTP hydrolysis rate of RagA 10-fold. As a potential molecular model that would be consistent with our data, MORTOR-mediated amino acid signaling may result from the assembly of a persistent complex between RagA-GTP, mTORC1 and MORTOR that slows RagA GTP hydrolysis.

Evolution and Cellular Roles of the MORTOR/Ragulator/Rag/mTORC1 Signaling System

Our systems analysis of mTORC1 signaling argues that the mTORC1 module functions as a broad integrator of metabolic, proliferation, and stress pathways. These pathways are among the most critical in evolution, allowing cells and organisms to survive under restrictive conditions. This central role of mTORC1 also elevates many of its direct regulators into putative drug targets that may be useful in the treatment of metabolic diseases as well as in cancer, obesity, aging and neurodegenerative diseases (Zoncu, R., et al. (2011) “mTOR: from Growth Signal Integration to Cancer, Diabetes and Ageing,” Nature Reviews Molecular Cell Biology, 12:21-35; Zoncu, R., et al. (2011) “MTORC1 Senses Lysosomal Amino Acids Through an Inside-Out Mechanism that Requires the Vacuolar H(+)-ATPase,” Science, 334:678-683). Given that MORTOR has a GPCR-like structure, this new mTORC1 regulator provides an interesting new drug target.

While the preferred embodiments of the invention have been illustrated and described, it will be appreciated that various changes can be made therein without departing from the spirit and scope of the invention.

1-3. (canceled)

4. A method for treating a subject having a disease or condition associated with dysregulation of mammalian target of rapamycin complex 1 (mTORC1), the method comprising administering a therapeutically effective amount of an antagonist of mediator of amino acid signaling to mTOR (MORTOR) to the subject.

5. The method of claim 4, wherein the antagonist is an antagonist of GPR137B.

6. The method of claim 4, wherein the antagonist is an antagonist of GPR137A.

7. The method of claim 4, wherein the antagonist is an antagonist of GPR137C.

8. (canceled)

9. The method of claim 4, wherein the antagonist downregulates expression of MORTOR through RNA interference (RNAi).

10. The method of claim 4, wherein the antagonist is an antisense oligonucleotide or inhibitory RNA comprising a nucleotide sequence sufficiently complementary to a MORTOR target mRNA sequence to bind to and downregulate expression of MORTOR.

11. The method of claim 10, wherein the MORTOR mRNA comprises a sequence selected from the group consisting of SEQ ID NO:1 and SEQ ID NO:2.

12. The method of claim 10, wherein the antagonist is selected from the group consisting of a small interfering RNA (siRNA), a short hairpin RNA (shRNA), a microRNA (miRNA), a Piwi-interacting RNA (piRNA), a small nuclear RNA (snRNA), and an antisense oligonucleotide.

13. The method of claim 12, wherein the antagonist is an siRNA comprising a sequence selected from the group consisting of SEQ ID NO:3 and SEQ ID NO:4.

14. The method of claim 8, wherein the disease or condition associated with dysregulation of mTORC1 is cancer.

15. The method of claim 4, wherein the antagonist downregulates expression of MORTOR through Clustered Regularly Interspaced Short Palindromic Repeats (CRISPR) interference (CRISPRi).

16. The method of claim 15, wherein the antagonist comprises one or more nucleic acids encoding a CRISPRi crRNA and a nuclease-deficient Cas protein, wherein the crRNA binds to a MORTOR mRNA comprising a sequence selected from the group consisting of SEQ ID NO:1 and SEQ ID NO:2.

17. (canceled)

18. The method of claim 4, wherein the antagonist is an antibody that specifically binds to MORTOR.

19. The method of claim 18, wherein the antibody specifically binds to a MORTOR protein comprising a sequence selected from the group consisting of SEQ ID NOS:5-7 or a sequence having at least 90% identity to a sequence selected from the group consisting of SEQ ID NOS:5-7.

20. (canceled)

21. The method of claim 4, wherein the subject is a mammal.

22. The method of claim 21, wherein the subject is human.

23. (canceled)

24. The method of claim 4, wherein the antagonist is administered locally into a tumor.

25. A method for inhibiting cell proliferation, the method comprising introducing an effective amount of a MORTOR antagonist into a cell.

26. The method of claim 25, wherein the cell is a cancerous cell.

27. The method of claim 25, wherein the antagonist downregulates expression of MORTOR through RNA interference (RNAi).

28. The method of claim 27, wherein the antagonist is an antisense oligonucleotide or inhibitory RNA comprising a nucleotide sequence sufficiently complementary to a MORTOR target mRNA sequence to bind to and downregulate expression of the MORTOR mRNA.

29. The method of claim 28, wherein the MORTOR mRNA comprises a sequence selected from the group consisting of SEQ ID NO:1 and SEQ ID NO:2.

30. The method of claim 28, wherein the antagonist is selected from the group consisting of a small interfering RNA (siRNA), a short hairpin RNA (shRNA), a microRNA (miRNA), a Piwi-interacting RNA (piRNA), a small nuclear RNA (snRNA), and an antisense oligonucleotide.

31. The method of claim 30, wherein the antagonist is an siRNA comprising a sequence selected from the group consisting of SEQ ID NO:3 and SEQ ID NO:4.

32. The method of claim 25, wherein the antagonist downregulates expression of MORTOR through Clustered Regularly Interspaced Short Palindromic Repeats (CRISPR) interference (CRISPRi).

33. The method of claim 32, wherein the antagonist comprises one or more nucleic acids encoding a CRISPRi crRNA and a nuclease-deficient Cas protein, wherein the crRNA binds to a MORTOR mRNA comprising a sequence selected from the group consisting of SEQ ID NO:1 and SEQ ID NO:2.

34. (canceled)

35. The method of claim 25, wherein the antagonist is an antibody that specifically binds to MORTOR.

36. The method of claim 35, wherein the antibody specifically binds to a MORTOR protein comprising a sequence selected from the group consisting of SEQ ID NOS:5-7 or a sequence having at least 90% identity to a sequence selected from the group consisting of SEQ ID NOS:5-7.

37. (canceled)

38. A method for inhibiting MORTOR in a subject comprising administering an effective amount of an antagonist of MORTOR to the subject.

39. A method of decreasing translocation of mTORC1 to lysosomes in a cell, the method comprising introducing an effective amount of an antagonist of MORTOR into the cell.

40-59. (canceled)
